(19) United States
(12) Patent Application Publication
(10) Pub. No.: US 2010/0273776 A1
(43) Pub. Date: Oct. 28, 2010

(54) INHIBITION OF ALPHA-SYNUCLEIN TOXICITY

(75) Inventors: Susan L. Lindquist, Chestnut Hill, MA (US); Tiago Outeiro, Cambridge, MA (US); Richard Labaudinière, Sherborn, MA (US); James Fleming, Somerville, MA (US); Christine Ellen Bulawa, Arlington, MA (US); Charlotte Weigel, Melrose, MA (US); Feng Liang, Sudbury, MA (US); Sandeep Gupta, Concord, OH (US); Amy Ripka, Winthrop, MA (US)

Correspondence Address: FISH & RICHARDSON P.C., P.O. BOX 1022, MINNEAPOLIS, MN, 55440-1022, US

(73) Assignees: FOLDRx PHARMACEUTICALS, INC, Cambridge, MA (US); WHITEHEAD INSTITUTE FOR BIOMEDICAL RESEARCH, Cambridge, MA (US)

(21) Appl. No.: 12/294,893

(22) PCT Filed: Mar. 29, 2007
(86) PCT No.: PCT/US07/07607

§ 371 Date: Jun. 29, 2010

Related U.S. Application Data
(60) Provisional application No. 60/787,113, filed on Mar. 29, 2006.

(51) Int. Cl. A61K 31/519; A61K 31/4045; A61K 31/416; A61K 31/454; A61K 31/445; A61K 31/435; A61K 31/55; C07D 487/04; C07D 209/14; C07D 231/56; C07D 471/04; A61P 43/00; A61P 25/16; A61P 25/28; C12N 5/07

(52) U.S. Cl. 514/215; 514/262.1; 514/415; 514/406; 514/303; 514/319; 514/297; 544/262; 548/511; 548/361.1; 546/119; 546/120; 544/280; 435/375

(57) ABSTRACT

Compounds and compositions are provided for treatment or amelioration of one or more symptoms of α-synuclein toxicity, α-synuclein mediated diseases or diseases in which α-synuclein fibrils are a symptom or cause of the disease.

### CROSS-REFERENCE TO RELATED APPLICATIONS

This application claims the benefit of U.S. Provisional Patent Application Ser. No. 60/787,113, filed Mar. 29, 2006, the entire content of which is incorporated herein by reference.

### STATEMENT AS TO FEDERALLY SPONSORED RESEARCH

Funds used to support some of the studies disclosed herein were provided by grant number NIH NS 44829 awarded by the National Institutes of Health. The Government may have certain rights in the invention.

### FIELD

The subject matter provided herein relates to compounds, composition and methods of inhibiting α-synuclein toxicity. The compounds can be used in methods of treatment of α-synuclein fibril mediated diseases, such as Parkinson's disease.

### BACKGROUND

Parkinson's disease is a neurodegenerative disorder that is pathologically characterized by the presence of intracytoplasmic Lewy bodies (Lewy in Handbuch der Neurologie, M. Lewandowski, ed., Springer, Berlin, pp. 920-933, 1912; Pollanen et al., J. Neuropath. Exp. Neurol. 52:183-191, 1993), the major components of which are filaments consisting of α-synuclein (Spillantini et al., Proc. Natl. Acad. Sci. USA 95:6469-6473, 1998; Arai et al., Neurosci. Lett. 259:83-86, 1999), an 140-amino acid protein (Ueda et al., Proc. Natl. Acad. Sci:USA 90:11282-11286, 1993). Two dominant mutations in α-synuclein causing familial early onset Parkinson's disease have been described suggesting that Lewy bodies contribute mechanistically to the degeneration of neurons in Parkinson's disease and related disorders (Polymeropoulos et al., Science 276:2045-2047, 1997; Kruger et al., Nature Genet. 18:106-108, 1998; Zarranz et al., Ann. Neurol. 55:164-173, 2004). Triplication and duplication mutation of the α-synuclein gene have been linked to early-onset of Parkinson's disease (Singleton et al., Science 302:841, 2003; Chartier-Harlin at al. Lancet 364:1167-1169, 2004; Ibanez et al., Lancet 364:1169-1171, 2004). In vitro studies have demonstrated that recombinant α-synuclein can indeed form Lewy body-like fibrils (Conway et al., Nature Med. 4:1318-1320, 1998; Hashimoto et al., Brain Res. 799:301-306, 1998; Nahri et al., J. Biol. Chem. 274:9843-9846, 1999). Both Parkinson's disease-linked α-synuclein mutations accelerate this aggregation process, demonstrating that such in vitro studies may have relevance for Parkinson's disease pathogenesis. α-synuclein aggregation and fibril formation fulfills of the criteria of a nucleation-dependent polymerization process (Wood et al., J. Biol. Chem. 274:19509-19512, 1999). In this regard α-synuclein fibril formation resembles that of Alzheimer's β-amyloid protein (Aβ) fibrils. α-synuclein recombinant protein, and non-Aβ component (known as NAC), which is a 35-amino acid peptide fragment of α-synuclein, both have the ability to form fibrils when incubated at 37° C., and are positive with amyloid stains such as Congo red (demonstrating a red/green birefringence when viewed under polarized light) and Thioflavin S (demonstrating positive fluorescence) (Hashimoto et al., Brain Res. 799:301-306, 1998; Ueda et al., Proc. Natl. Acad. Sci. USA 90:11282-11286, 1993).

Synucleins are a family of small, presynaptic neuronal proteins composed of α-, β-, and γ-synucleins, of which only α-synuclein aggregates have been associated with several neurological diseases (Ian et al., Clinical Neurosc. Res. 1:445-455, 2001; Trojanowski and Lee, Neurotoxicology 23:457-460, 2002). The role of synucleins (and in particular, α-synuclein) in the etiology of a number of neurodegenerative and/or amyloid diseases has developed from several observations. Pathologically, α-synuclein was identified as a major component of Lewy bodies, the hallmark inclusions of Parkinson's disease, and a fragment thereof was isolated from amyloid plaques of a different neurological disease, Alzheimer's disease. Biochemically, recombinant α-synuclein was shown to form amyloid-like fibrils that recapitulated the ultrastructural features of α-synuclein isolated from patients with dementia with Lewy bodies, Parkinson's disease and multiple system atrophy. Additionally, the identification of mutations within the α-synuclein gene, albeit in rare cases of familial Parkinson's disease, demonstrated an unequivocal link between synuclein pathology and neurodegenerative diseases. The common involvement of α-synuclein in a spectrum of diseases such as Parkinson's disease, dementia with Lewy bodies, multiple system atrophy and the Lewy body variant of Alzheimer's disease has led to the classification of these diseases under the umbrella term of “synucleinopathies.”

Fibrillization and aggregation of α-synuclein is thought to play major role in neuronal dysfunction and death of dopaminergic neurons in PD. Mutations in α-synuclein or genomic triplication of wild type α-synuclein (leading to its overexpression) cause certain rare familial forms of Parkinson's disease. In vitro and in vivo models suggest that over-expression of wild-type α-synuclein induces neuronal cell death. See, e.g., Polymeropoulos, et al. (1997) Science 276(5321):2045-7, Kruger, et al. (1998) Nat. Genet. 18(2):106-8, Singleton, et al. (2003) Science 302(5646):841, Miller, et al. (2004) Neurology 62(10):1835-8, Hashimoto, et al. (2003) Ann N Y Acad. Sci. 991:171-88, Lo Bianco, et al. (2002) Proc Natl Acad Sci USA. 99(16):10813-8, Lee, et al. (2002) Proc Natl Acad Sci USA. 99(13):8968-73, Masliah, et al. (2000) Science 287(5456):1265-9, Auluck, et al. (2002) Science 295(5556):865-8, Oluwatosin-Chigbu et al. (2003) Biochem Biophys Res Commun 309(3): 679-84, Klucken et al. (2004) J Biol. Chem. 279(24):25497-502. Protecting neurons from the toxic effects of α-synuclein is a promising strategy for treating Parkinson's disease and other synucleinopathies such as Lewy body dementia.

Thus, there is a need for compounds and compositions that prevent α-synuclein toxicity and/or aggregation and/or promote α-synuclein fibril disaggregation. Such compounds and composition are useful in treating or ameliorating one or more symptoms of α-synuclein mediated diseases and disorders, or diseases and disorders in which a-synuclein fibril formation is implicated, including but not limited to, Parkinson's disease, dementia with Lewy bodies, multiple system atrophy and the Lewy body variant of Alzheimer's disease.

### SUMMARY

Provided herein are compounds, compositions containing the compounds, and methods of use of the compounds as α-synuclein inhibitors. Also provided are methods of treatment or amelioration of one or more symptoms of diseases and disorders associated with α-synuclein toxicity. Also provided are methods of treatment or amelioration of one or more symptoms of diseases and disorders associated with α-synuclein fibril formation. Such diseases and disorders include, but are not limited to, Parkinson's disease and Lewy body dementia. Other diseases and disorders include tauopathies, such as, but not limited to, Alzheimer's disease.

Use of any of the described compounds for the treatment or amelioration of one or more symptoms of diseases and disorders associated with α-synuclein toxicity or α-synuclein fibril formation is also contemplated. Furthermore, use of any of the described compounds for the manufacture of a medicament for the treatment of diseases and disorders associated with α-synuclein toxicity or α-synuclein fibril formation is also contemplated.

In various embodiments, the compounds for use in the compositions and methods provided herein are according to Formula I:

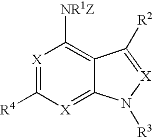

or pharmaceutically acceptable salts or derivatives thereof, wherein:

m is 1 or 2;

n is 0, 1, 2, or 3;

each X is independently N or CH;

R¹ and Z are each independently R⁵, C(O)R⁵, COOR⁵, C(O)NR⁵R⁵, or S(O)ₘR⁵;

R² and R³ are each independently H, halo, pseudohalo, CN, SR⁵, R⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, or NR⁵C(O)C(O)NR⁵R⁶;

R⁴ is independently H; halo, pseudohalo, CN, SR⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, or NR⁵C(O)C(O)NR⁵R⁶; or optionally substituted alkyl, aryl, aralkyl, heteroaryl, or heteroaralkyl; and

each R⁵, R⁶, and R⁸ is independently H or optionally substituted alkyl, alkenyl, alkynyl, cycloalkyl, cycloalkenyl, aryl, heteroaryl, or heterocyclyl.

In various embodiments, R¹ is H.

In various embodiments, the compound is represented by Formula Ib or Ic

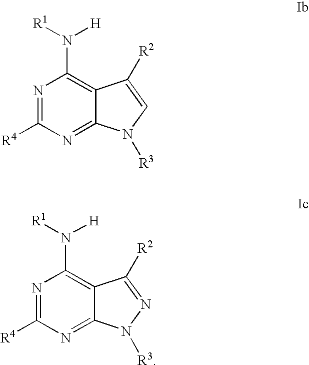

In various embodiments, R² is H, halo, CN, NO₂, NH₂, or C₁-C₁₀ alkyl optionally substituted with 1-3 independent halo, SR⁵, OR⁵, OC(O)R⁵, NR⁵R⁵; COOR⁵, NO₂, CN, C(O)R⁵, OC(O)NR⁵R⁵, or C(O)NR⁵R⁵. In some embodiments, R² is H, F, Cl, Br, CF₃, CCl₃, CN, NO₂, NH₂, or C₁-C₆ alkyl. In some embodiments, R² is aryl, heteroaryl, aralkyl, or heteroaralkyl, each substituted with: H, halo, SR⁵, OR⁵, OC(O)R⁵, NR⁵R⁵; COOR⁵, NO₂, CN, C(O)R⁵, OC(O)NR⁵R⁵, or C(O)NR⁵R⁵; or aryl, C₁-C₁₀ alkyl, or C₂-C₁₀ alkenyl each optionally substituted with 1-3 independent aryl, halo, SR⁵, OR⁵, OC(O)R⁵, NR⁵R⁵; COOR⁵, NO₂, CN, C(O)R⁵, OC(O)NR⁵R⁵, or C(O)NR⁵R⁵. The optionally substituted aryl, heteroaryl, aralkyl, or heteroaralkyl groups in R² may be as described in the Detailed Description, or may be selected, for example, from phenyl, napthyl, benzyl, phenylethylene, napthylmethylene, phenoxymethylene, napthyloxymethylene, pyridylmethylene, benzofurylmethylene, dihydrobenzofurylmethylene, benzodioxolmethylene, indanylmethylene, furyl, thienyl, pyridyl, benzothienyl, and benzofuryl. The optional substituents for the aryl, heteroaryl, aralkyl, or heteroaralkyl groups in R² may be as described in the Detailed Description, or in some embodiments may be selected from: H, F, Cl, Br, OH, C₁-C₆ alkoxy, amino, C₁-C₆ alkylamino, COOH, COO—C₁-C₆ alkyl, NO₂, CN, or C(O)—C₁-C₆ alkyl; or C₁-C₆ alkyl, C₂-C₆ alkenyl, or aryl optionally substituted with phenyl, F, Cl, Br, C₁-C₆ alkoxy, COOH, COO—C₁-C₆ alkyl, NO₂, or CN.

In various embodiments, R² is phenyl, napthyl, benzofuryl, benzothienyl, furyl, or thienyl, each optionally substituted with: halo, CN, amino, alkylamino, C₁-C₆ hydroxyalkyl, S—C₁-C₆ alkyl, C₁-C₆ alkoxy, C₁-C₆ haloalkoxy, COOH, COO—C₁-C₆ alkyl, C(O)—C₁-C₆ alkyl, or C₃-C₆ cycloalkyl; or optionally halogenated aryl, aralkyl, O-aryl, or O-aralkyl. In some embodiments, R² is optionally substituted phenyl, napthyl, benzofuryl, benzothienyl, furyl, thienyl, fluoronapthyl, benzyloxyphenyl, (chlorobenzyl)oxyphenyl, hydroxymethylphenyl, cyclohexylphenyl, chorophenyl, cyanophenyl, carboxyl phenyl, alkyl carboxyl phenyl, alkanoyl phenyl, alkylamino phenyl, trifluoromethoxyphenyl, alkoxyphenyl, phenoxyphenyl, biphenyl, or alkyl-S-phenyl. In some embodiments, R² is aralkyl, aralkenyl, or heteroaralkyl, each optionally substituted with halo, CN, amino, alkylamino, S—C₁-C₆ alkyl, C₁-C₆ alkoxy, C₁-C₆ haloalkoxy, C₁-C₆ haloalkyl, C₂-C₆ alkynyl, aryl, haloaryl, or heteroaryl. In some embodiments, R² is CH₂, CH(CH₃), CH═CH, or CH₂CH₂, each substituted with phenyl, naphthyl, tetrahydronaphthyl, pyridyl, indanyl, benzofuryl, benzodioxolyl, dihydrobenzofuranyl, or tetrahydronaphthyl, wherein each phenyl, napthyl, tetrahydronaphthyl, pyridyl, indanyl, benzofuryl, benzodioxolyl, dihydrobenzofuranyl, or tetrahydronaphthyl in R² is optionally substituted with one or two substituents selected from the group consisting of F, Cl, CF₃; C₁-C₆ alkyl, C₁-C₆ alkoxy, acetylenyl, CN, alkylamino, and phenyl. In certain embodiments, R² is CH(CH₃)-phenyl, CH═CH-phenyl, CH₂CH₂-phenyl, CH₂-naphthyl, CH₂-(methylnaphthyl), CH₂-(fluoronaphthyl), CH₂-pyridyl, CH₂-indanyl, CH₂-benzofuryl, CH₂-benzodioxolyl, CH₂-dihydrobenzofuranyl, CH₂-tetrahydronaphthyl, dichlorobenzyl, (chloro,trifluoromethyl)benzyl, (fluoro,trifluoromethyl)benzyl, (fluoro,chloro)benzyl, dimethylbenzyl, (methyl,fluoro)benzyl, dimethoxybenzyl, (acetylenyl)benzyl, cyanobenzyl, (dimethylamino)benzyl, methoxybenzyl, or phenylbenzyl.

In various embodiments, R³ is H; C₁-C₁₀ alkyl or C₂-C₁₀ alkenyl each optionally substituted with 1-3 halo, CF₃, SR⁵, OR⁵, OC(O)R⁵, NR⁵R⁵; COOR⁵, NO₂, CN, C(O)R⁵, OC(O)NR⁵R⁵, C(O)NR⁵R⁵; C₃-C₁₀ cycloalkyl; or C₂-C₁₀ alkynyl. In some embodiments, R³ is H, C₁-C₈ alkyl optionally substituted with 1-3 halo, OR⁵, NR⁵R⁵, COOR⁵, C(O)R⁵, C(O)NR⁵R⁵, C₂-C₆ alkenyl, or C₂-C₆ alkynyl; or cyclopropyl, cyclopropylmethyl, cyclobutyl, cyclobutylmethyl, cyclopentyl, cyclopentylmethyl, cyclohexyl, or cyclohexylmethyl. In certain embodiments, R³ is aryl, heteroaryl, aralkyl, heteroaralkyl, heterocyclyl, or heterocyclyalkyl, each substituted with: H, alkyl, halo, OR⁵, OC(O)R⁵, NR⁵R⁵; COOR⁵, NO₂, CN, C(O)R⁵, OC(O)NR⁵R⁵, or C(O)NR⁵R⁵; or optionally substituted aryl, heteroaryl, or heterocyclyl. The aryl, heteroaryl, aralkyl, heteroaralkyl, heterocyclyl, or heterocyclyalkyl groups represented by R³ may be as described in the Detailed Description or can be selected, for example, from benzyl, pyridyl, pyridylmethylene, furyl, thienyl, tetrahydrofuryl, or tetrahydrothienyl. The substituents for the aryl, heteroaryl, aralkyl, heteroaralkyl, heterocyclyl, or heterocyclyalkyl groups represented by R³ may be as described in the Detailed Description, or can be selected from, for example: H, F, Cl, Br, SR⁵, OR⁵, NR⁵R⁵; COOR⁵, NO₂, CN, C(O)R⁵; or C₁-C₆ alkyl, C₂-C₆ alkenyl, or aryl optionally substituted with phenyl, F, Cl, Br, SR⁵, OR⁵, COOR⁵, NO₂, or CN.

In various embodiments, R³ is optionally substituted aryl; C₁-C₁₀ optionally substituted with aryl or C₃-C₁₀ cycloalkyl; C₃-C₁₀ cycloalkyl; C₂-C₁₀ alkenyl, or C₂-C₁₀ alkynyl. In some embodiments, R³ is optionally substituted propenyl, propynyl, benzyl, cyclobutyl, cyclopropylmethyl, 2,2-dimethylpropyl, cyclohexyl, cyclopentyl, cyclopropyl, phenylethylene, ethyl, 2-propyl, methyl, phenyl, nitrophenyl, sec-butyl, or tert-butyl.

In various embodiments, R⁴ is independently aryl; heteroaryl; C₁-C₁₀ alkyl or C₂-C₁₀ alkenyl, each optionally substituted with 1-3 independent aryl, R⁷, or heteroaryl; C₂-C₁₀ alkynyl; halo; haloalkyl; CF₃; SR⁵; OR⁵; OC(O)R⁵; NR⁵R⁵; NR⁵R⁶; COOR⁵; NO₂; CN; C(O)R⁵; C(O)C(O)R⁵; C(O)NR⁵R⁵; S(O)ₘR⁵; S(O)ₘNR⁵R⁵; NR⁵C(O)NR⁵R⁵; NR⁵C(O)C(O)R⁵; NR⁵C(O)R⁵; NR⁵(COOR⁵); NR⁵C(O)R⁸; NR⁵S(O)ₘNR⁵R⁵; NR⁵S(O)ₘR⁵; NR⁵S(O)ₘR⁸; NR⁵C(O)C(O)NR⁵R⁵; or NR⁵C(O)C(O)NR⁵R⁶. In some embodiments, R⁴ is: H; OR⁵; OC(O)R⁵; NR⁵R⁵; COOR⁵; NO₂; CN; C(O)R⁵; C(O)C(O)R⁵; or C(O)NR⁵R⁵; or C₁-C₁₀ alkyl optionally substituted with 1-3 halo, OR⁵, OC(O)R⁵, NR⁵R⁵; COOR⁵, NO₂, CN, C(O)R⁵, OC(O)NR⁵R⁵, or C(O)NR⁵R⁵. In certain embodiments, R⁴ is an optionally substituted aryl, aralkyl, heteroaryl, or heteroaralkyl, wherein the aryl, aralkyl, heteroaryl, or heteroaralkyl groups may be as described in the Detailed Description or can be selected, for example, from phenyl, benzyl, pyridyl, pyridylmethylene, furyl, furylmethylene, thienyl, thienylmethylene, pyrazolyl, and pyrazolylmethylene. The optional substituents for the optionally substituted aryl, aralkyl, heteroaryl, or heteroaralkyl groups represented by R⁴ may be as described in the Detailed Description, or can be selected from, for example: H, CF₃, CCl₃, amino, C₁-C₆ alkoxy, COOH, COO—C₁-C₆ alkyl, OC(O)—C₁-C₆ alkyl, phenoxy, or alkylphenoxy; or C₁-C₆ alkyl optionally substituted with amino, COOH, COO—C₁-C₆ alkyl or OC(O)—C₁-C₆ alkyl, or 1 or 2 C₁-C₆ alkoxy. In some embodiments, the optional substituents are halo, CF₃, SR⁵, OR⁵, OC(O)R⁵, NR⁵R⁵; COOR⁵, NO₂, CN, C(O)R⁵, OC(O)NR⁵R⁵, C(O)NR⁵R⁵, N(R⁵)C(O)R⁵, N(R⁵)(COOR⁵), or S(O)ₘNR⁵R⁵. In certain embodiments, the optional substituents are F, Cl, OH, amino, NO₂, C₁-C₆ alkoxy, C₁-C₆ alkyl, phenoxy, or alkylphenoxy; or phenyl, imidazolyl, or morpholino optionally substituted with F, Cl, amino, NO₂, C₁-C₆ alkoxy, or C₁-C₆ alkyl.

In various embodiments, wherein R⁴ is independently amino, alkylamino, or aryl, heteroaryl, or C₁-C₁₀ alkyl optionally substituted with halo, CF₃, O—C₁-C₆ alkyl, or aryloxy. In some embodiments, R⁴ is pyridyl, C₁-C₆ alkoxy-C₁-C₆ alkyl, (C₁-C₆ alkyl)phenoxy-C₁-C₆ alkyl, C₁-C₆ alkyl, amino, or halophenyl. In certain embodiments, R⁴ is pyridyl, CH(OCH₂CH₃)₂, tert-butyl-phenoxymethylene, methyl, ethyl, amino, or chlorophenyl. In some embodiments, R⁴ is pyridyl or C₁-C₆ alkyl. In some embodiments, R⁴ is pyridyl, methyl, or ethyl.

In various embodiments, the compound is selected from the compounds in FIGS. 1a, 1b, 1 c, 1d, 1e, or 1f. In some embodiments, the compound is selected from the compounds in FIGS. 1a, 1b, or 1f. In certain embodiments, the compound is selected from the compounds in FIGS. 1a and 1b, 1b and 1f, 1a and 1f, 1a, 1b, or 1f. In various embodiments, the compounds do not include the compounds of one or more of FIGS. 1c, 1d, and/or 1e; for example, in some embodiments, the compound is not a compound in FIGS. 1c, 1d, or 1e. In some embodiments, the compounds do not include the compounds of one or more of FIGS. 1c, 1d, 1e, and/or 1f.

In various embodiments, when R¹ and Z are H, R² is 5-NO₂-fur-2-yl, or phenyl optionally substituted with a single 4-Cl, 4-CH₃, or 4-OCH₃; and R³ is unsubstituted phenyl, cyclohexyl, or acyclic C₁-C₄ alkyl; and the compound is in the form of a free base; then R⁴ is not H, unsubstituted C₁-C₄ alkyl, or phenyl optionally substituted with 4-Cl or 4-CH₃. In various embodiments, when R¹ and Z are H, R² is CN or CH₂CN; and R³ is CH₃, or phenyl optionally substituted with 4-NO₂; then R⁴ is not CO₂-alkyl or CCl₃. In various embodiments, when R¹ and Z are H, R³ is cyclopentyl, and R⁴ is unsubstituted 4-pyridyl, then R² is not CF₃; CN, Br, Cl, or NO₂. In various embodiments, when R¹ and Z are H, R³ is cyclopentyl, and R⁴ is optionally substituted 4-pyridyl, then R² is not C₁-C₄ alkyl optionally substituted with F. In various embodiments, when R¹ and Z are H, R³ is unsubstituted C₁-C₄ alkyl, cyclopentyl, or phenyl, and R⁴ is unsubstituted pyridyl, then R² is not unsubstituted CH₃, benzyl, or CH₂-pyrid-4-yl, and then R² is not H when the compound is in the form of a free base. In various embodiments, when R¹ and Z are H, R² is H or unsubstituted C₁-C₂ alkyl, benzyl, or CH₂-pyridyl; and R⁴ is unsubstituted 4-pyridyl, then R³ is not a lone pair, C₁-C₄ alkyl optionally substituted with CO₂-alkyl, dialkylamino, or cyclopentyl; benzyl optionally substituted with Cl, CN, or CH₃; unsubstituted cyclobutyl, cyclopentyl, 3-tetrahydrofuryl, or 2-bicyclo[2.2.1]heptyl; and R³ is not H when the compound is in the form of a free base. In various embodiments, when R¹ and Z are H, R³ is H, a lone pair, cyclopentyl, 3-(5-ethyl-5H-[1,2,4]triazino[5,6-b]indolyl); unsubstituted benzyl; C₁-C₄ alkyl optionally substituted with OCH₃; phenyl optionally substituted with Cl, 3-NO₂, 4-NO₂, or 4-Me; or ribofuranose; and R⁴ is 2-furyl optionally substituted with 5-NO₂; 5-NH₂-pyrazol-4-yl optionally substituted with methyl or optionally chlorinated phenyl; phenyl optionally substituted with imidazolyl, 4-Cl, 4-OH, or 4-NO₂; C₁-C₄ alkyl optionally substituted with F or acetate; or unsubstituted benzyl; then R² is not unsubstituted C₁-C₂ alkyl, and when the compound is in the form of a free base, R² is not H. In various embodiments, when R¹ and Z are H, R³ is H or a lone pair, and R⁴ is phenyl optionally substituted with OH, NH₂, NO₂, NHC(O)NHPhSO₂F, NHC(O)PhSO₂F; fur-2-yl with an optional 5-NO₂ group, 3-NH₂-pyrazol-4-yl; C₁-C₄ alkyl optionally substituted with F or CO₂-alkyl; or unsubstituted pyridyl or benzyl; then R² is not CN, and R² is not H when the compound is in the form of a free base. In various embodiments, when R³ is tert-butyl; R⁴ is H; R¹ and Z are both H or acetyl, or R¹ is H and Z is acetyl, optionally substituted SO₂-phenyl, or substituted benzoyl; then R² is not H or Br; phenyl optionally 3 or 4-substituted with OCH₃, phenoxy or benzyloxy, or substituted only with a single Cl, 4-CF₃, 4-F, 4-C₁-C₄ alkyl, or 4-phenyl; benzyl optionally substituted with Cl, F, or CH₃; unsubstituted naphthyl, CH₂-naphthyl, or OCH₂-naphthyl; or unsubstituted thien-2-yl or benzothien-2-yl.

In some embodiments, when R¹ and Z are H, R² is nitrofuryl, or phenyl optionally substituted with halo, alkyl, or alkoxy; and R³ is unsubstituted alkyl, cycloalkyl, or phenyl; then R⁴ is not H, unsubstituted alkyl, or phenyl optionally substituted with Cl or alkyl. In some embodiments, when R¹ and Z are H, R² is CN or CH₂CN; and R³ is alkyl, or phenyl optionally substituted with NO₂; then R⁴ is not CO₂-alkyl or CCl₃. In some embodiments, when R¹ and Z are H, R³ is cycloalkyl, and R⁴ is optionally substituted pyridyl, then R² is not CF₃; CN, Br, Cl, or NO₂, or alkyl optionally substituted with F. In some embodiments, when R¹ and Z are H, R³ is unsubstituted alkyl, cycloalkyl, or phenyl, and R⁴ is unsubstituted pyridyl, then R² is not H or unsubstituted alkyl, benzyl, or CH₂-pyridyl. In some embodiments, when R¹ and Z are H, R² is H or unsubstituted alkyl, benzyl, or CH₂-pyridyl; and R⁴ is unsubstituted pyridyl, then R³ is not H, a lone pair, alkyl optionally substituted with CO₂-alkyl, dialkylamino, or cycloalkyl; benzyl optionally substituted with Cl, CN, or alkyl; unsubstituted cycloalkyl, bicycloalkyl, or tetrahydrofuryl. In some embodiments, when R¹ and Z are H, R² is H or unsubstituted alkyl, and R³ is H, a lone pair, cycloalkyl, a tricyclic heteroaryl substituted with alkyl; unsubstituted benzyl; C₁-C₄ alkyl optionally substituted with OCH₃; phenyl optionally substituted with C₁, NO₂, or Me; or ribofuranose; then R⁴ is not furyl optionally substituted with NO₂; NH₂-pyrazolyl optionally substituted with methyl or optionally chlorinated phenyl; phenyl optionally substituted with imidazolyl, Cl, OH, or NO₂; C₁-C₄ alkyl optionally substituted with F or acetate; or unsubstituted benzyl. In some embodiments, when R¹ and Z are H, R³ is H or a lone pair, and R² is H or CN, then R⁴ is not phenyl optionally substituted with OH, NH₂, NO₂, NHC(O)NHPhSO₂F, NHC(O)PhSO₂F; furyl optionally substituted with NO₂, NH₂-pyrazolyl; C₁-C₄ alkyl optionally substituted with F or CO₂-alkyl; or unsubstituted pyridyl or benzyl. In some embodiments, when R¹ and Z are both H or acetyl, or R¹ is H and Z is acetyl, SO₂-phenyl, or optionally substituted benzoyl, R³ is tert-butyl, and R⁴ is H, then R² is not H or Br; phenyl optionally substituted with Cl, CF₃, F, C₁-C₄ alkyl, phenyl, or OCH₃, phenoxy or benzyloxy; benzyl optionally substituted with Cl, F, or CH₃; unsubstituted naphthyl, CH₂-naphthyl, or OCH₂-naphthyl; or unsubstituted thienyl or benzothienyl.

In certain embodiments, when R¹ and Z are H, R² is nitrofuryl or optionally substituted phenyl; and R³ is unsubstituted alkyl, cycloalkyl, or phenyl; then R⁴ is not H, unsubstituted alkyl, or optionally substituted phenyl. In certain embodiments, when R¹ and Z are H, R² is CN or CH₂CN; and R³ is alkyl, or phenyl optionally substituted with NO₂; then R⁴ is not CO₂-alkyl or CCl₃. In certain embodiments, when R¹ and Z are H, R³ is unsubstituted alkyl, cycloalkyl, or phenyl, and R⁴ is optionally substituted pyridyl, then R² is not H oCF₃; CN, Br, C₁, NO₂, alkyl, haloalkyl, benzyl, or CH₂-pyridyl. In certain embodiments, when R¹ and Z are H, R² is H or unsubstituted alkyl, benzyl, or CH₂-pyridyl; and R⁴ is unsubstituted pyridyl, then R³ is not H, a lone pair, optionally substituted alkyl, dialkylamino, or cycloalkyl; optionally substituted benzyl; cycloalkyl, bicycloalkyl, or tetrahydrofuryl. In certain embodiments, when R¹ and Z are H, R² is H or alkyl, and R³ is H, a lone pair, cycloalkyl, a tricyclic heteroaryl substituted with alkyl; benzyl; alkyl, alkoxyalkyl; optionally substituted phenyl; or ribofuranose; then R⁴ is not optionally substituted furyl, NH₂-pyrazolyl, phenyl, alkyl or benzyl. In certain embodiments, when R¹ and Z are H, R³ is H or a lone pair, and R² is H or CN, then R⁴ is not an optionally substituted phenyl; furyl, pyrazolyl; alkyl, pyridyl or benzyl. In certain embodiments, when R¹ and Z are both H or acetyl, or R¹ is H and Z is acetyl, SO₂-phenyl, or optionally substituted benzoyl, R³ is tert-butyl, and R⁴ is H, then R² is not H or Br; optionally substituted phenyl, phenoxy, benzyloxy, benzyl, naphthyl, CH₂-naphthyl, OCH₂-naphthyl, thienyl or benzothienyl.

In some embodiments, when R¹ and Z are H, R² is nitrofuryl or optionally substituted phenyl; and R³ is alkyl, cycloalkyl, or phenyl; then R⁴ is not H, alkyl, or optionally substituted phenyl. In some embodiments, when R¹ and Z are H, R² is CN or CH₂CN; and R³ is alkyl or optionally substituted phenyl; then R⁴ is not CO₂-alkyl or CCl₃. In some embodiments, when R¹ and Z are H, R³ is unsubstituted alkyl, cycloalkyl, or phenyl, and R⁴ is optionally substituted pyridyl, then R² is not H, CN, Br, Cl, NO₂, alkyl, haloalkyl, benzyl, or CH₂-pyridyl. In some embodiments, when R¹ and Z are H, R² is H or unsubstituted alkyl, benzyl, or CH₂-pyridyl; and R⁴ is unsubstituted pyridyl, then R³ is not H, a lone pair, dialkylamino, or optionally substituted alkyl, cycloalkyl, bicycloalkyl, benzyl, or tetrahydrofuryl. In some embodiments, when R¹ and Z are H, R² is H or alkyl, and R³ is H, a lone pair, cycloalkyl, a substituted tricyclic heteroaryl, benzyl, alkyl, alkoxyalkyl; optionally substituted phenyl; or a sugar; then R⁴ is not optionally substituted furyl, pyrazolyl, phenyl, alkyl or benzyl. In some embodiments, when R¹ and Z are H, R³ is H or a lone pair, and R² is H or CN, then R⁴ is not an optionally substituted phenyl, furyl, pyrazolyl, alkyl, pyridyl or benzyl. In some embodiments, when R¹ and Z are both H or acetyl, or R¹ is H and Z is acetyl, SO₂-phenyl, or optionally substituted benzoyl, R³ is tert-butyl, and R⁴ is H, then R² is not H or Br; optionally substituted phenyl, phenoxy, benzyloxy, benzyl, naphthyl, CH₂-naphthyl, OCH₂-naphthyl, thienyl or benzothienyl.

In some embodiments, the compound is one of:

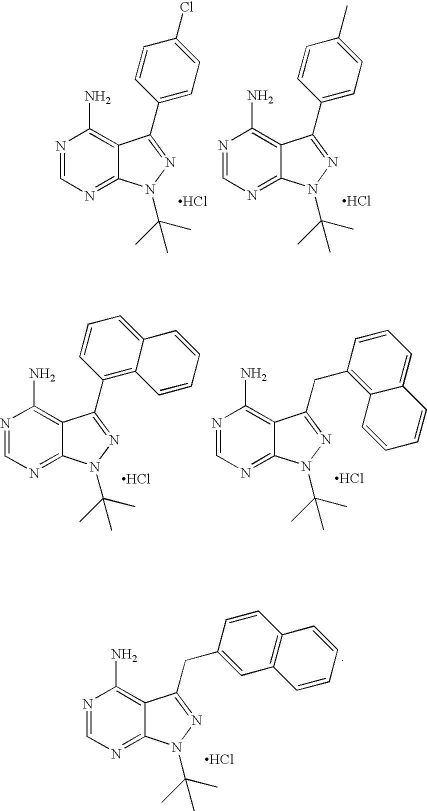

In various embodiments, the compounds for use in the compositions and methods provided herein are according to Formula I^{o}:

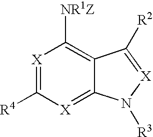

or pharmaceutically acceptable salts or derivatives thereof, where:

n can be 0, 1, 2, or 3;

R² can be H, halo, pseudohalo, (CH₂)—Y, or (CH═CH)ₙ—Y, where Y can be unsubstituted or substituted aryl, heteroaryl, alkyl, or cycloalkyl;

R³ can be substituted or unsubstituted alkyl, alkenyl, alkynyl, aryl, aralkyl, cycloalkyl, (CH₂)ₙ-cycloalkyl, or adamantyl;

R⁴ can be H, NH₂, NR⁵R⁶, NR⁵COR⁶, or unsubstituted or substituted alkyl or aryl;

R¹, Z, R⁵, and R⁶ can be independently selected from H, unsubstituted or substituted alkyl, aralkyl, aryl, alkaryl, or cycloalkyl, COR^{o}^{⁷}, where R^{o}^{⁷} is unsubstituted or substituted alkyl or aryl, SO₂R^{o}^{⁸}, where R^{o}^{⁸} is aryl or substituted aryl, and (CH₂)ₙ-cycloalkyl, where the cycloalkyl may be substituted; and

X can be CH or N.

In some embodiments, possible substituents for Y can be selected from halo, pseudohalo, alkyl, cycloalkyl, aryl, aralkyl, NO₂, alkoxy, aryloxy, arylalkyoxy, CF₃, OCF₃, CN, NR⁵R⁶, NR⁵COR⁶, (CH₂)ₙOR⁶, SR⁶, CO₂H, CO₂R⁶, CONR⁶R⁵, COR⁶, and SO₂NR⁵R⁶.

In some embodiments, possible substituents for R⁴ include halo, alkyl, cycloalkyl, aryl, aralkyl, NO₂, alkoxy, aryloxy, arylalkyoxy, CF₃, OCF₃, CN, NR⁵R⁶, NR⁵COR⁶, (CH₂)ₙOR⁶, SR⁶, CO₂H, CO₂R⁶, CONR⁶R⁵, COR⁶, and SO₂NR⁵R⁶. In some embodiments, substituents for R⁴ groups are halo or alkyl.

In some embodiments, n is 1. In some embodiments, n is 0.

In some embodiments, each X is N.

In some embodiments, R¹ and Z are each independently hydrogen, or substituted or unsubstituted alkyl, arylcarbonyl, aralkylcarbonyl, haloarylcarbonyl, arylsulfonyl, aralkylsulfonyl, or haloarylsulfonyl.

In some embodiments, R¹ and Z are each independently hydrogen, methyl, COR^{o}^{⁷}, where R^{o}^{⁷} is methyl, phenyl, tolyl, 2-chlorophenyl, or 4-fluorophenyl, or SO₂R^{o}^{⁸}, where R^{o}^{⁸} is phenyl, tolyl, or 4-chlorophenyl. In some embodiments, R¹ is H and Z is H. In some embodiments, R¹ is methyl and Z is H.

In some embodiments, R² is hydrogen, halo, or substituted or unsubstituted aryl, heteroaryl, aralkyl, or aralkenyl.

In some embodiments, R² is hydrogen, bromo, phenyl, tolyl, styrenyl, benzyl, naphthyl, naphthylmethyl, 4-biphenyl, 3-methylphenyl, 4-ethylphenyl, 4-isopropylphenyl, 4-(n-butyl)phenyl, 4-tert-butylphenyl, 4-cyclohexylphenyl, 2-methoxyphenyl, 4-methoxyphenyl, 2-chlorophenyl, 3-chlorophenyl, 4-chlorophenyl, 3,4-dichlorophenyl, 4-fluorophenyl, 3,4-difluorophenyl, 4-cyanophenyl, 4-trifluoromethylphenyl, 3-trifluoromethoxyphenyl, 3-methyl-4-fluorophenyl, 4-hydroxymethyl-phenyl, 4-(dimethylamino)phenyl, 4-(ethoxycarbonyl)phenyl, 4-(hydroxycarbonyl)-phenyl, 4-(phenoxy)phenyl, 4-(2-naphtylmethyl)-phenyl, 2-furyl, 3-furyl, 2-thienyl, 3-thienyl, 2-benzofuryl, 4-acetophenone, or 2-benzothienyl.

In some embodiments, R³ is substituted or unsubstituted alkyl, cycloalkyl, aryl, or aralkyl.

In some embodiments, R³ is methyl, ethyl, isopropyl, tert-butyl, 2-dimethylpropyl, 2-propenyl, 2-propynyl, 2-methylbutyl, cyclobutyl, cyclopentyl, cyclohexyl, cyclopropylmethyl, phenyl, or benzyl.

In various embodiments, R⁴ is hydrogen, amino, or substituted or unsubstituted aryl. In some embodiments, R⁴ is hydrogen, amino, tolyl, or 4-chlorophenyl. In some embodiments, R⁴ is H. In some embodiments, R⁴ is amino.

Also provided are pharmaceutically-acceptable derivatives, including salts, esters, enol ethers, enol esters, solvates, hydrates and prodrugs of the compounds described herein.

Further provided are pharmaceutical compositions containing the compounds provided herein and a pharmaceutically acceptable carrier. In various embodiments, the pharmaceutical compositions are formulated for single dosage administration.

Also provided are methods of treating or ameliorating one or more symptoms of α-synuclein-mediated diseases or disorders. Such diseases and disorders include, but are not limited to, Parkinson's disease, dementia with Lewy bodies, multiple system atrophy and the Lewy body variant of Alzheimer's disease.

Method of treating or ameliorating one or more symptoms associated with α-synuclein toxicity are provided. Methods of prevention of α-synuclein fibril formation are provided. Methods of disruption or disaggregation of α-synuclein fibrils are provided. In further embodiments, methods of restoring vesicle trafficking and/or reversing changes in lipid metabolism are provided. In another embodiment, methods of slowing or reversing or ameliorating neurodegeneration are provided.

In practicing the methods, effective amounts of the compounds or compositions containing therapeutically effective concentrations of the compounds are administered.

Articles of manufacture are provided containing packaging material, a compound or composition provided herein which is useful for treating or ameliorating one or more symptoms of α-synuclein-mediated diseases or disorders, and a label that indicates that the compound or composition is useful for treating or ameliorating one or more symptoms of α-synuclein-mediated diseases or disorders.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1a and 1b set forth the structures for certain compounds, e.g., according to Formula I^{o} or Formula I, as described herein.

FIGS. 1c and 1d set forth the structures for certain compounds.

FIG. 1e sets forth the structures for certain free base compounds.

FIG. 1f sets forth the structures for certain compounds as hydrochloride salts

FIGS. 2-4 demonstrated dose-dependent activity of five compounds described herein in a yeast α-syn toxicity inhibition assay. Compounds were serially diluted into wells containing the α-syn expressing strain in minimal medium containing 0.1 M MOPS, pH 6.0. After 24 hours at 30° C., growth was determined by measuring OD600. See also Example 1.

### DETAILED DESCRIPTION

### A. Definitions

Unless defined otherwise, all technical and scientific terms used herein have the same meaning as is commonly understood by one of ordinary skill in the art to which this invention belongs. All patents, applications, published applications and other publications are incorporated by reference in their entirety. In the event that there are a plurality of definitions for a term herein, those in this section prevail unless stated otherwise.

As used herein, α-synuclein refers to one in a family of structurally related proteins that are prominently expressed in the central nervous system. Aggregated α-synuclein proteins form brain lesions that are hallmarks of some neurodegenerative diseases (synucleinopathies). The gene for α-synuclein, which is called SNCA, is on chromosome 4q21. One form of hereditary Parkinson disease is due to mutations in SNCA. Another form of hereditary Parkinson disease is due to a triplication of SNCA.

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs.

Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl.

Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

Also included in the present invention are pharmaceutically acceptable salts of the disclosed compounds. These disclosed compounds can have one or more sufficiently acidic protons that can react with a suitable organic or inorganic base to form a base addition salt. When it is stated that a compound has a hydrogen atom bonded to an oxygen, nitrogen, or sulfur atom, it is contemplated that the compound also includes salts thereof where such a hydrogen atom has been reacted with a suitable organic or inorganic base to form a base addition salt. Base addition salts include those derived from inorganic bases, such as ammonium or alkali or alkaline earth metal hydroxides, carbonates, bicarbonates, and the like, and organic bases such as alkoxides, alkyl amides, alkyl and aryl amines, and the like. Such bases useful in preparing the salts of this invention thus include sodium hydroxide, potassium hydroxide, ammonium hydroxide, potassium carbonate, and the like.

For example, pharmaceutically acceptable salts of the disclosed compounds can include those formed by the reaction of the disclosed compounds with one equivalent of a suitable base to form a monovalent salt (i.e., the compound has single negative charge that is balanced by a pharmaceutically acceptable counter cation, e.g., a monovalent cation) or with two equivalents of a suitable base to form a divalent salt (e.g., the compound has a two-electron negative charge that is balanced by two pharmaceutically acceptable counter cations, e.g., two pharmaceutically acceptable monovalent cations or a single pharmaceutically acceptable divalent cation). “Pharmaceutically acceptable” means that the cation is suitable for administration to a subject. Examples include alkali metal cations, such as but not limited Li⁺, Na⁺, K⁺; alkali earth metal cations, such as but not limited to Ba²⁺, Mg²⁺, Ca²⁺; transition metal cations, such as but not limited to Zn²⁺ and other metal salts; and NR₄⁺, wherein each R is independently hydrogen, an optionally substituted aliphatic group (e.g., a hydroxyalkyl group, aminoalkyl group or ammoniumalkyl group) or optionally substituted aryl group, or two R groups, taken together, form an optionally substituted non-aromatic heterocyclic ring optionally fused to an aromatic ring. For example, salts can be formed with amines including, but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane. In some embodiments, the pharmaceutically acceptable cation is Li⁺, Na⁺, K⁺, NH₃(C₂H₅OH)⁺ or N(CH₃)₃(C₂H₅OH)⁺.

Pharmaceutically acceptable salts of the disclosed compounds with a sufficiently basic group, such as an amine, can be formed by reaction of the disclosed compounds with an organic or inorganic acid to form an acid addition salt. Acids commonly employed to form acid addition salts from compounds with basic groups can include inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include nitrates, borates, trifluoroacetates, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, butyrates, valerates, isobutyrates, caproates, heptanoates, propiolates, oxalates, malonates, succinates, suberates, sebacates, fumarates, maleates, butyne-1,4-dioates, hexyne-1,6-dioates, ascorbates, salicylates, benzoates, chlorobenzoates, methylbenzoates, dinitrobenzoates, hydroxybenzoates, methoxybenzoates, phthalates, sulfonates, benzenesulfonates, toluenesulfonates, xylenesulfonates, phenylacetates, phenylpropionates, phenylbutyrates, citrates, lactates, gamma-hydroxybutyrates, glycolates, tartrates, methanesulfonates, propanesulfonates, naphthalene-1-sulfonates, naphthalene-2-sulfonates, mandelates, and the like. In certain embodiments, the disclosed compound forms a pharmaceutically acceptable salt with HCl, HF, HBr, HI, trifluoracetic acid, or sulfuric acid. In particular embodiments, the disclosed compound forms a pharmaceutically acceptable salt with sulfuric acid.

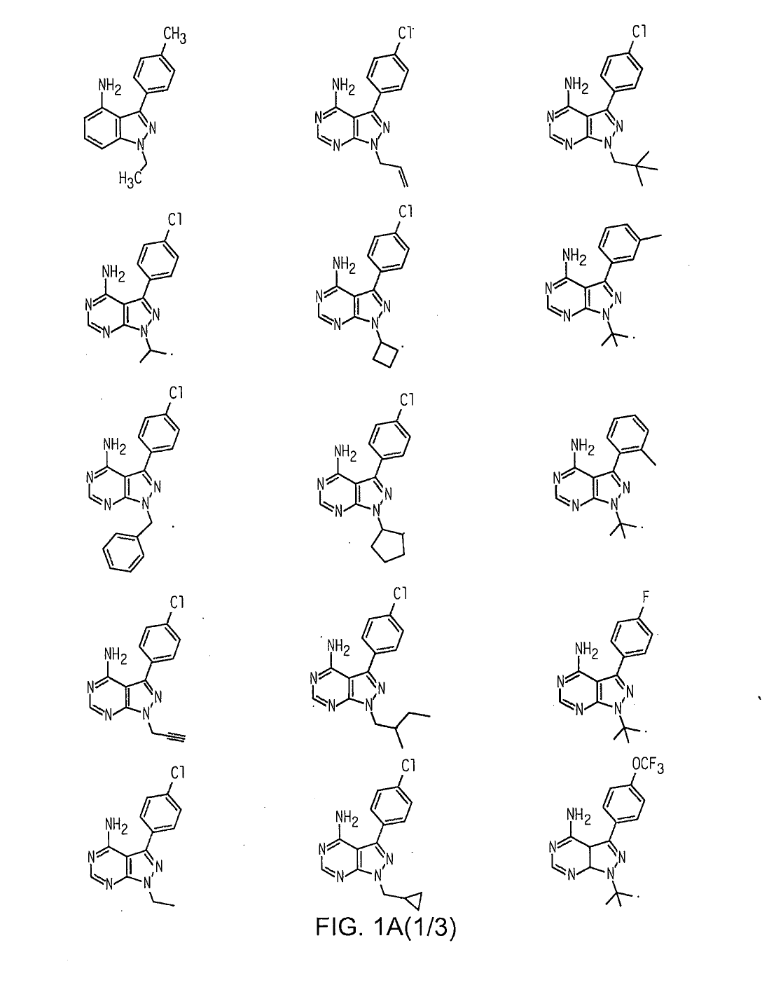

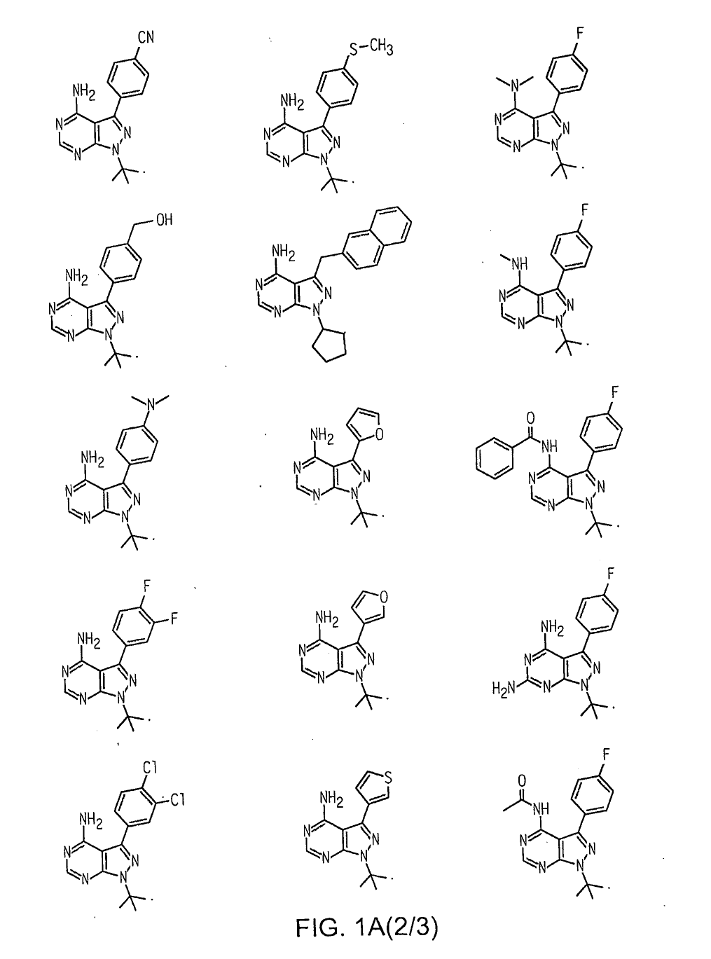

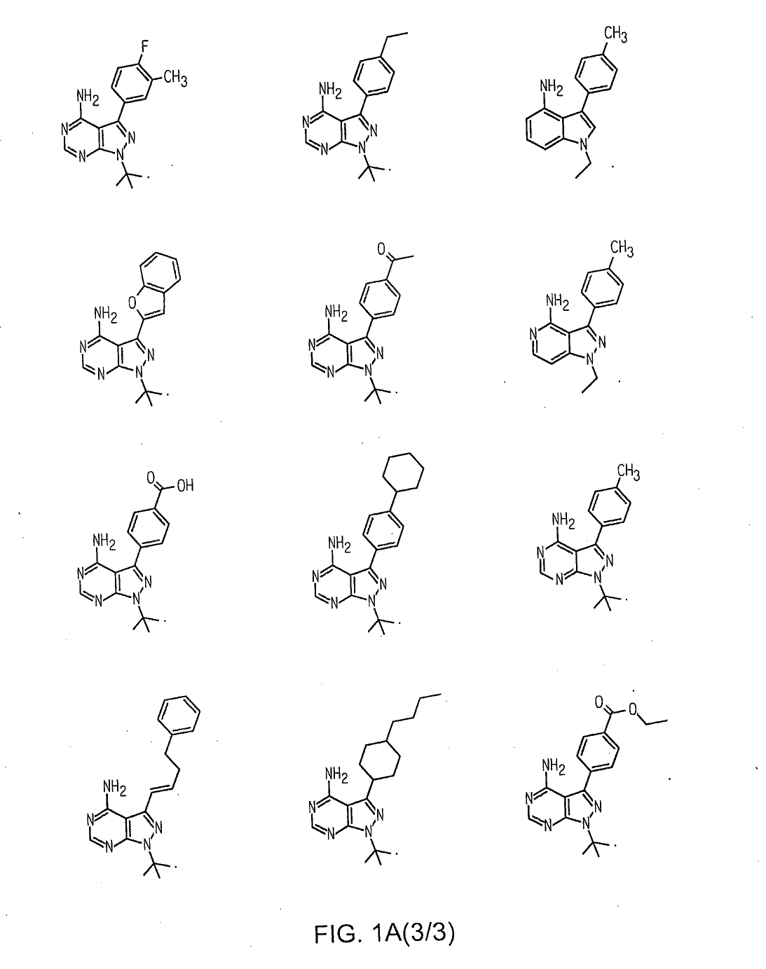

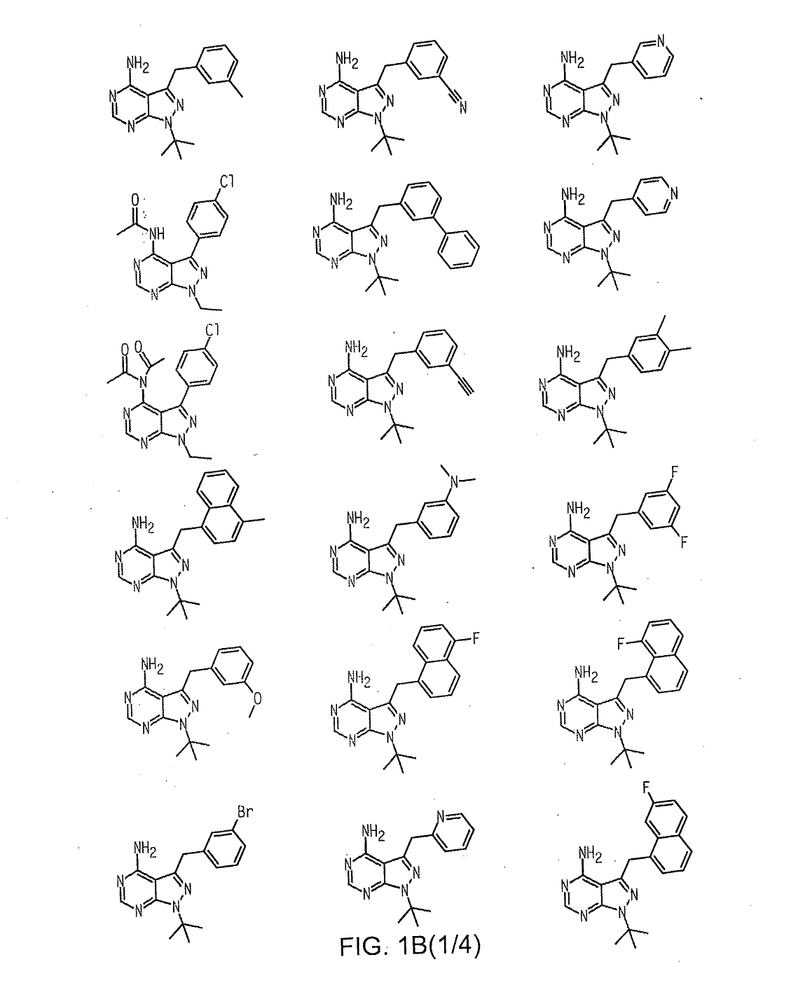

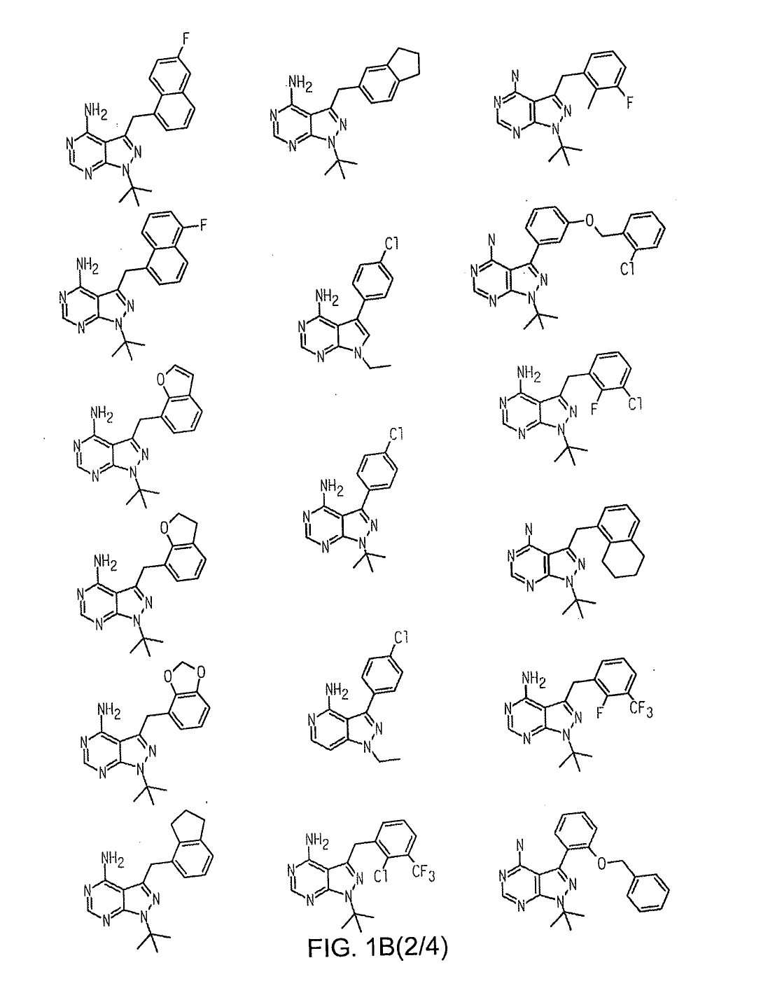

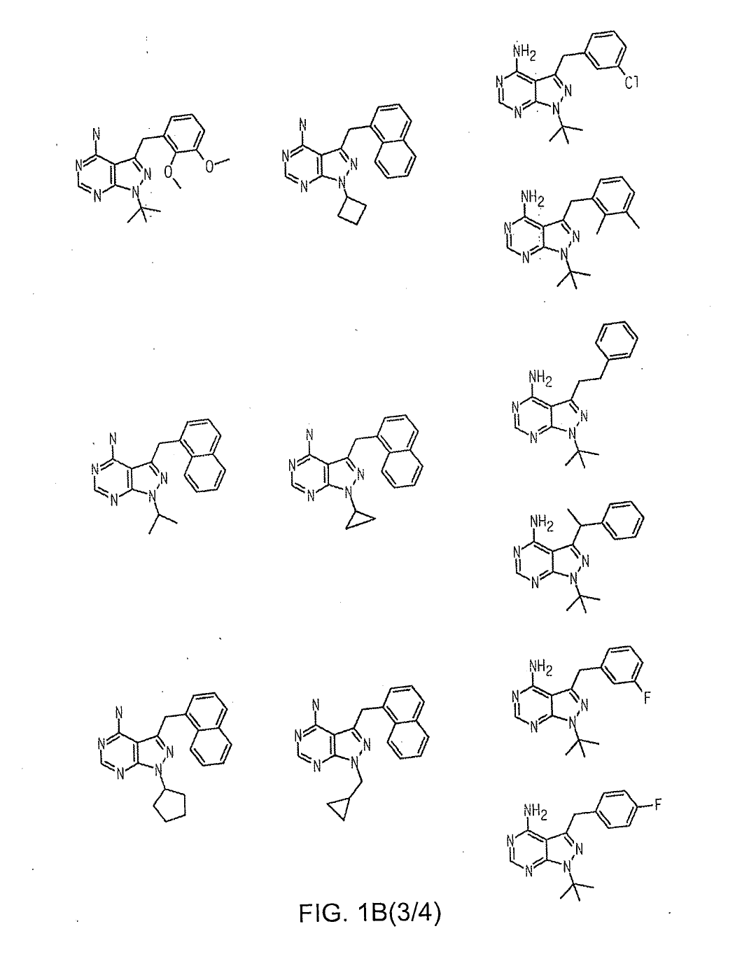

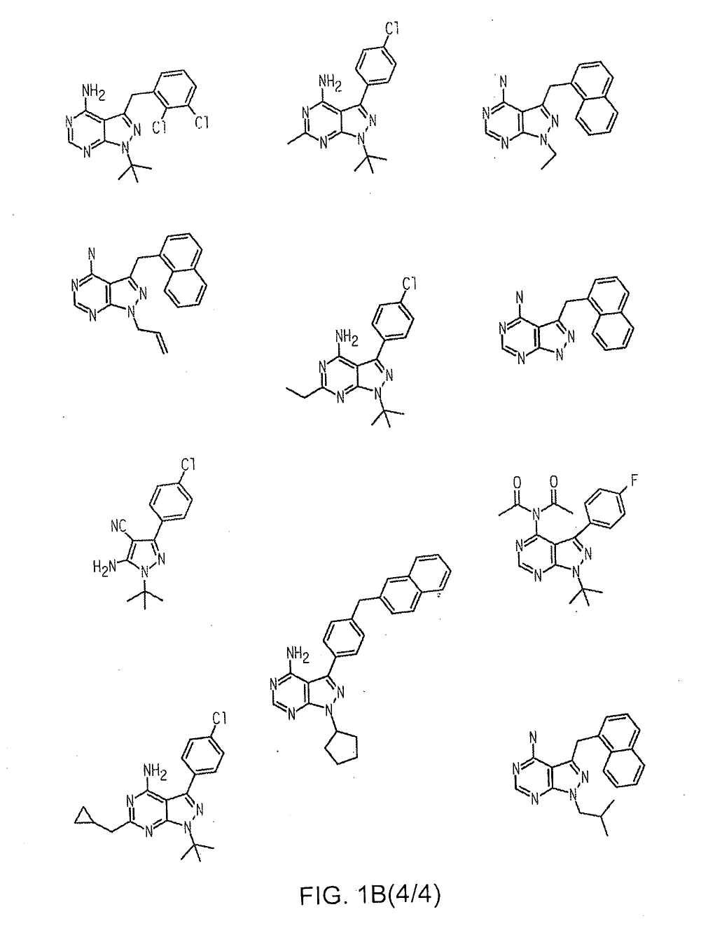

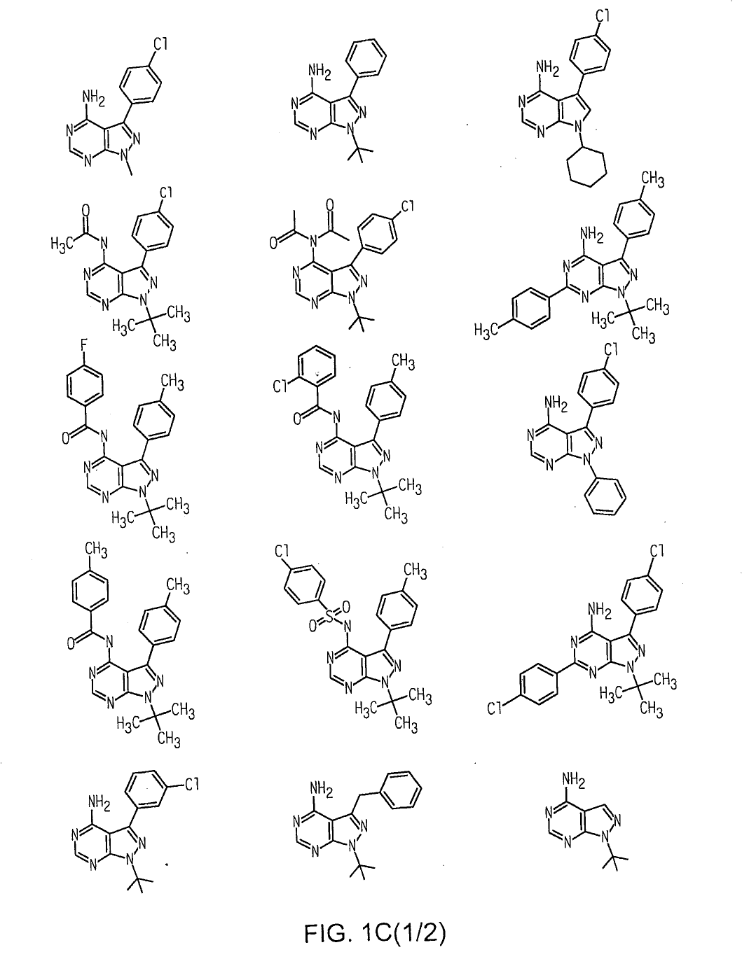

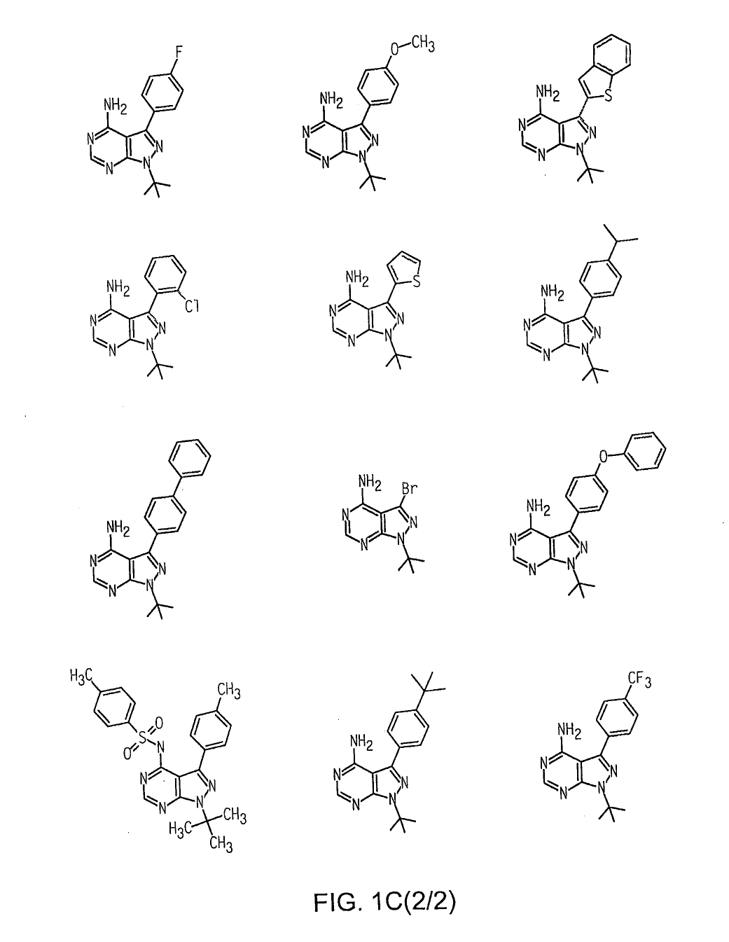

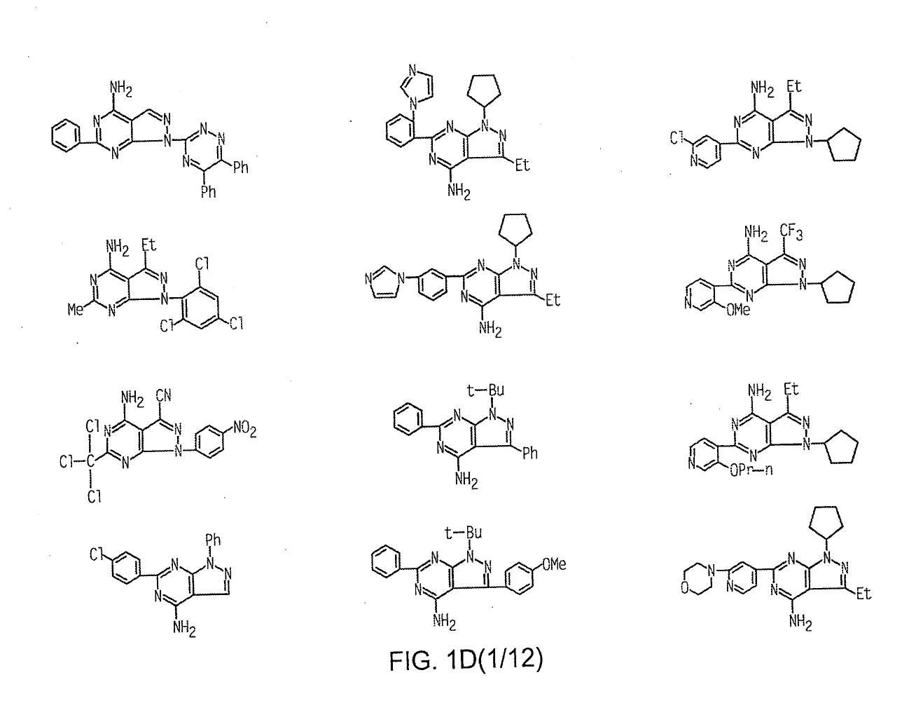

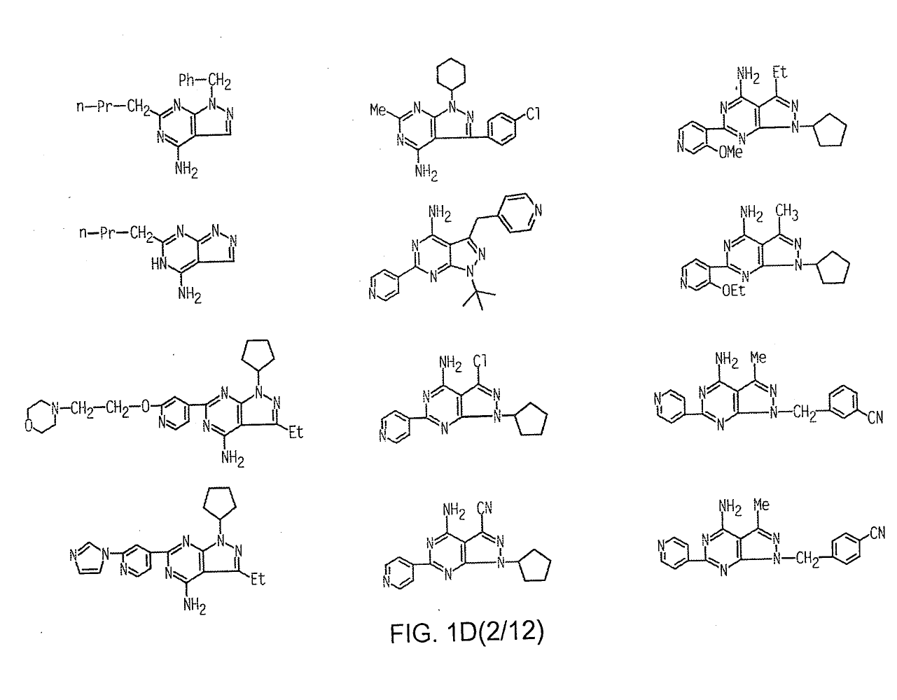

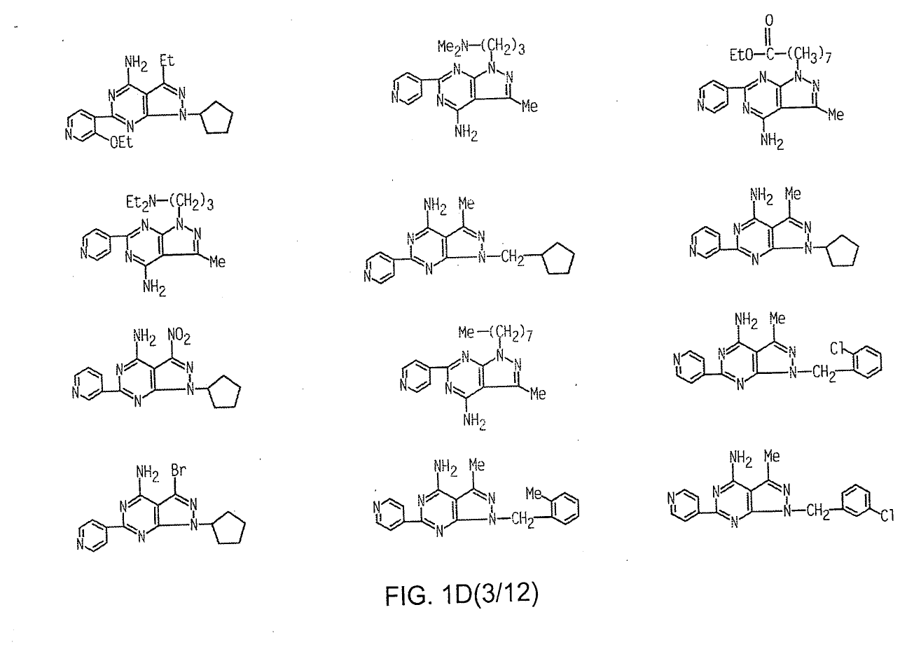

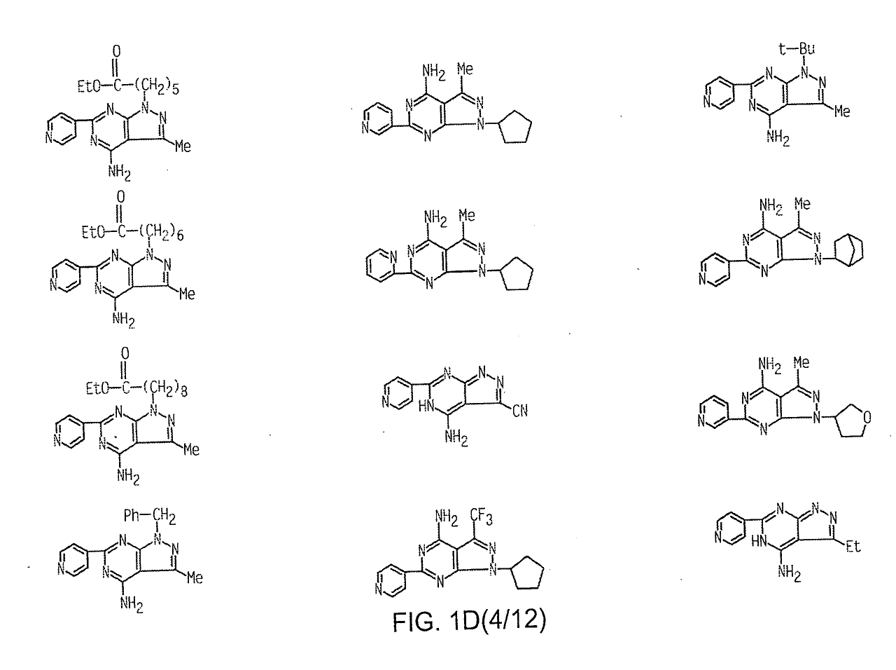

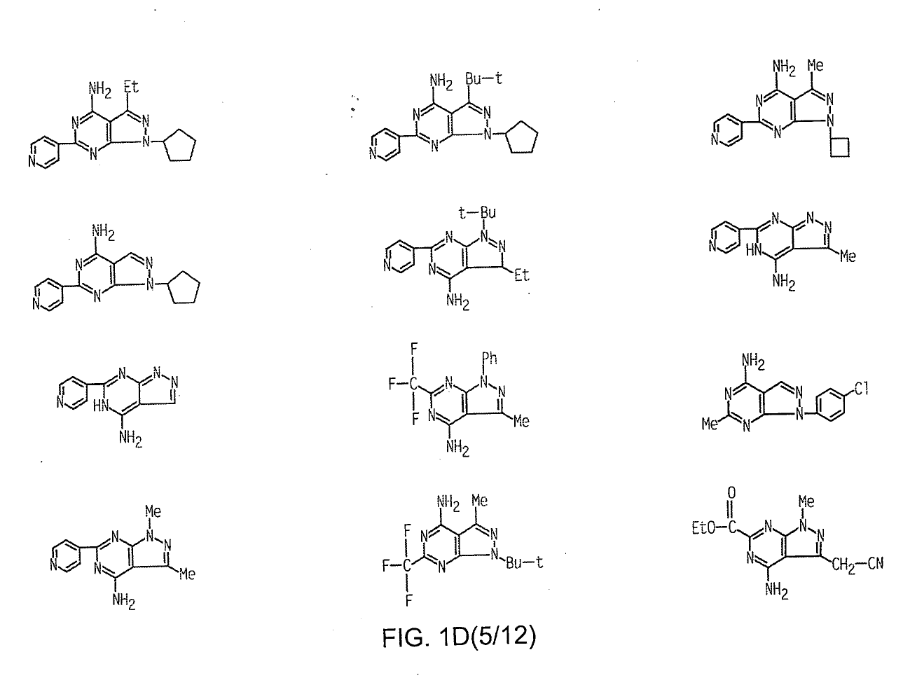

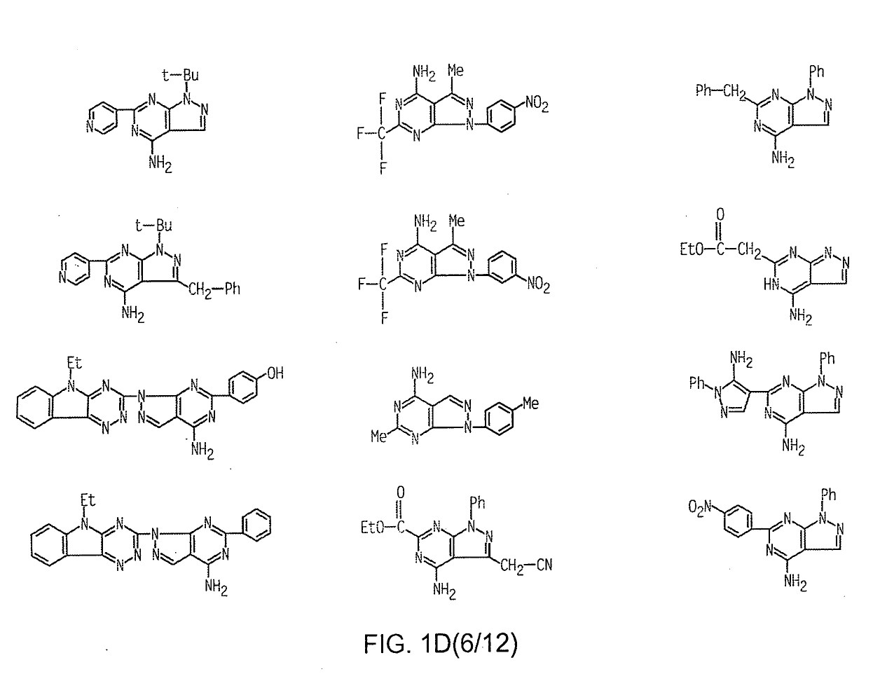

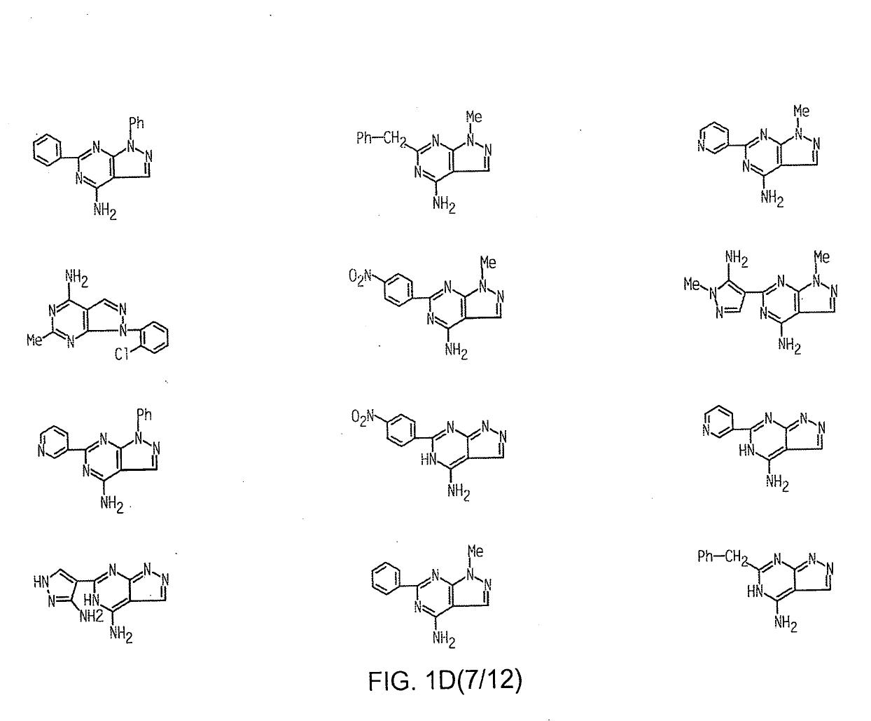

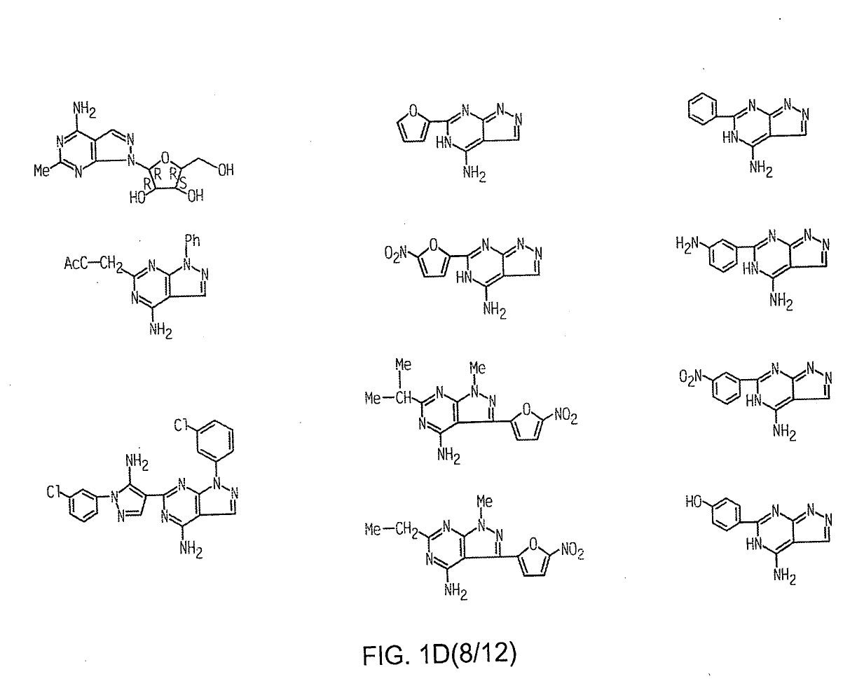

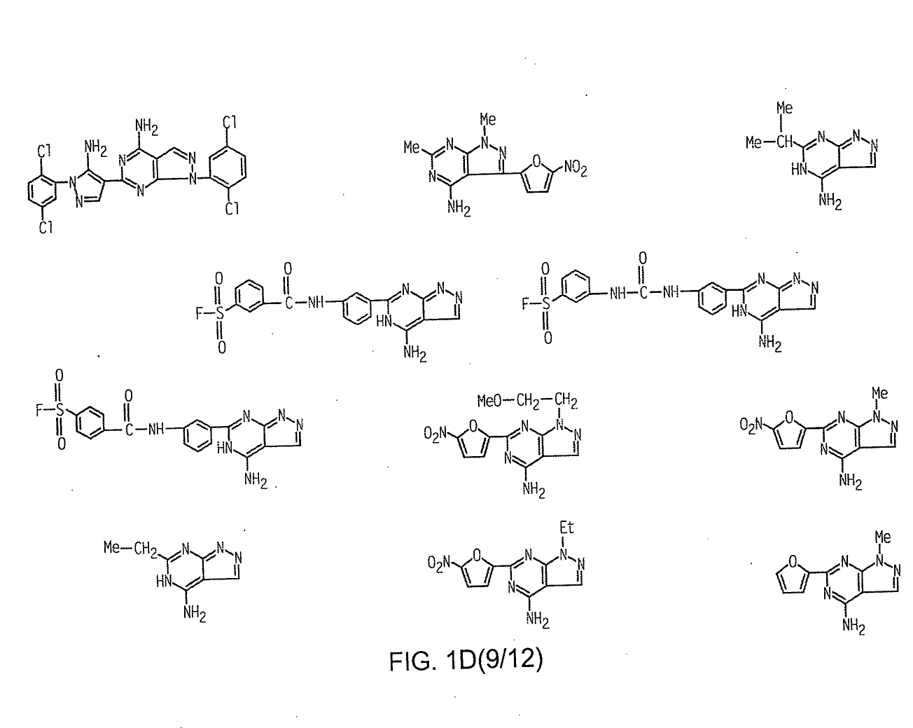

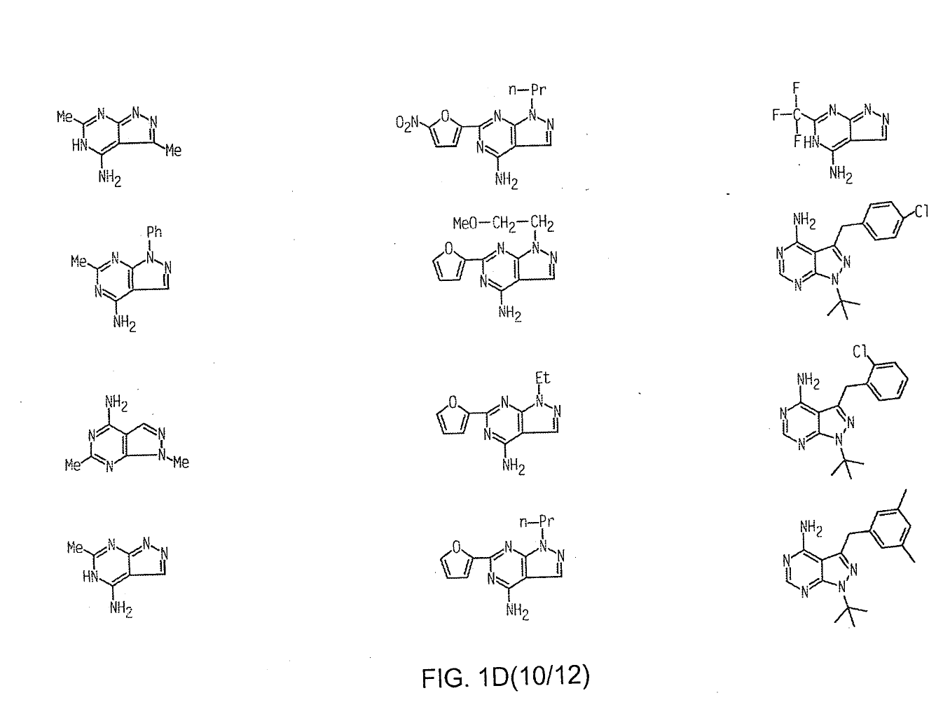

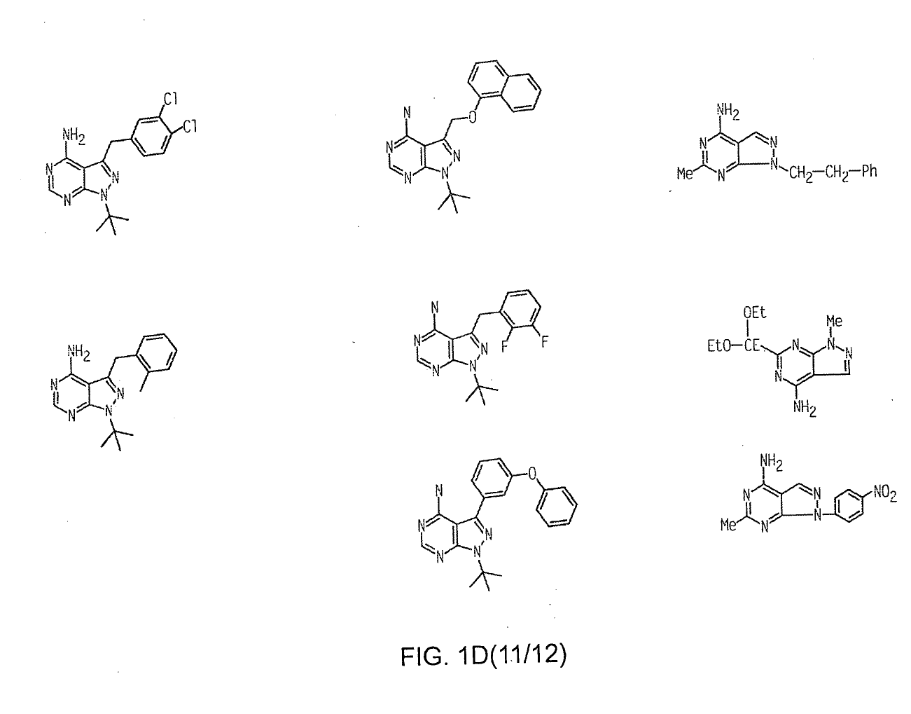

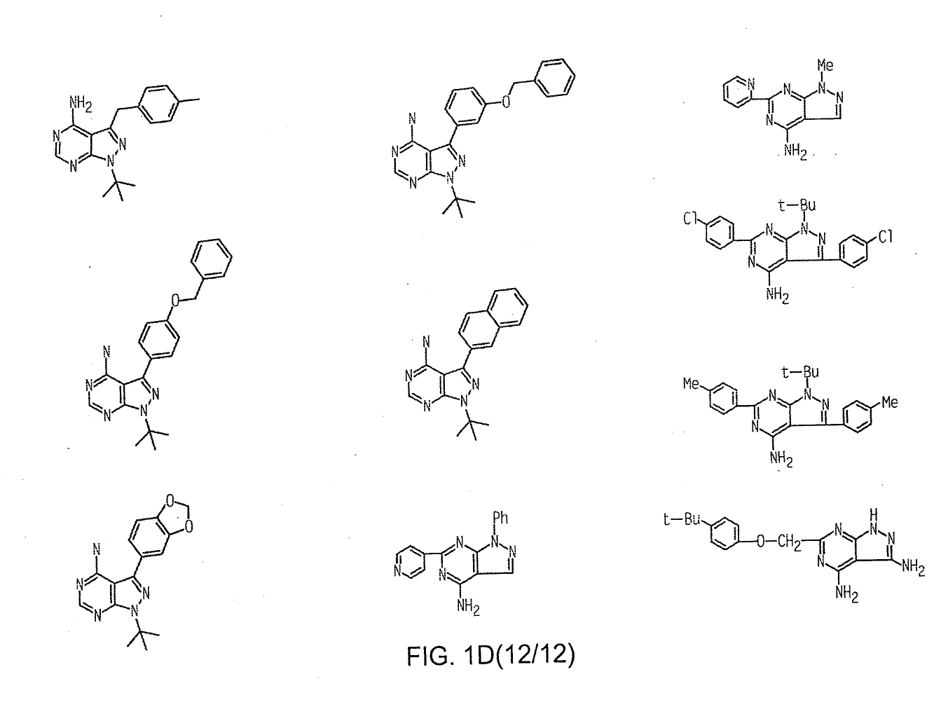

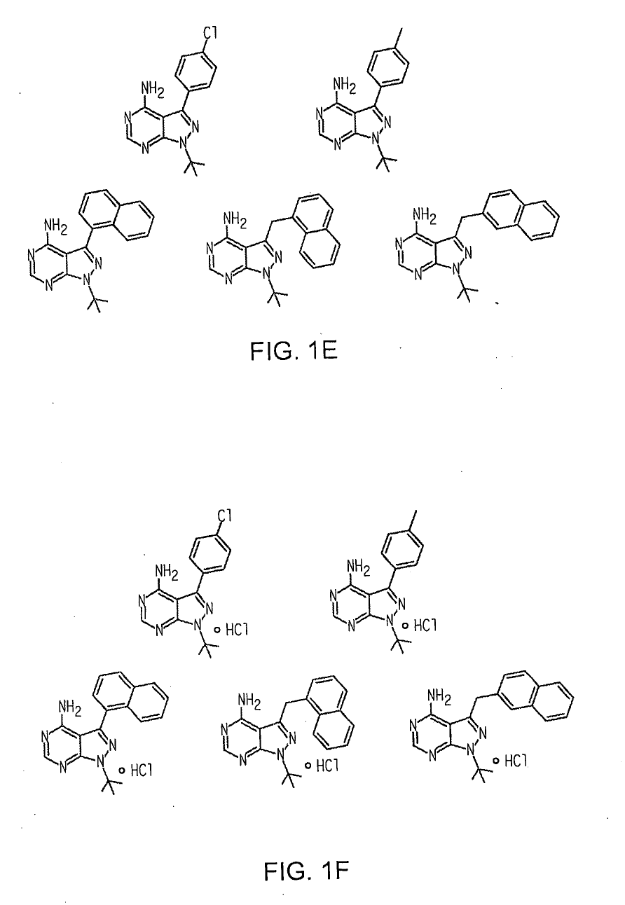

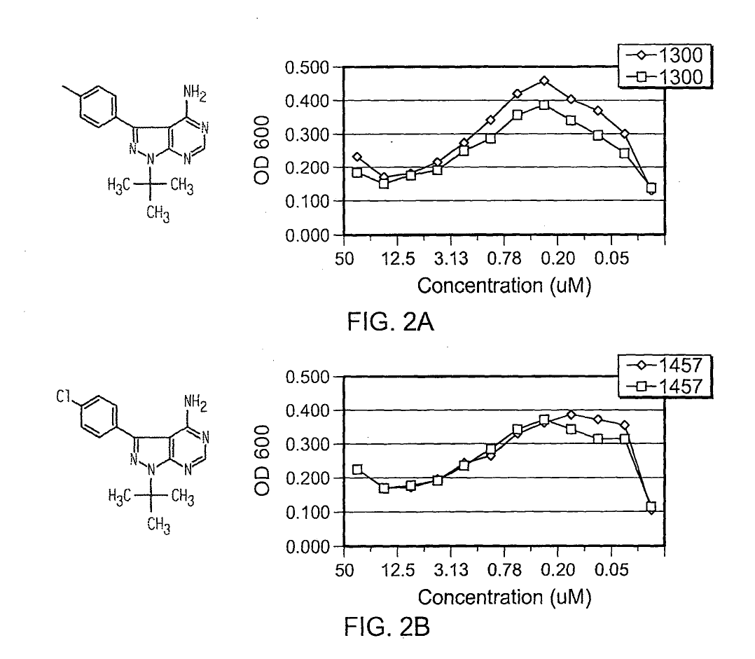

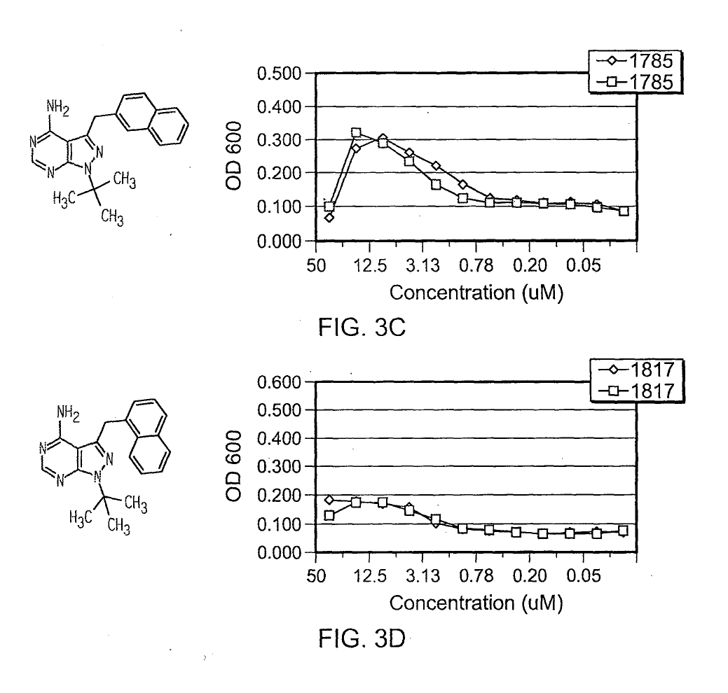

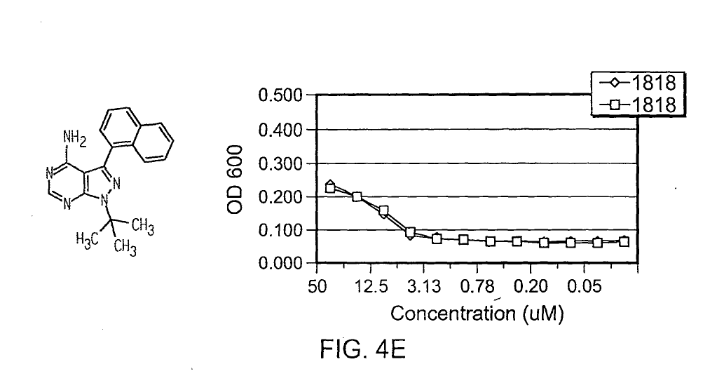

Various embodiments are directed to pharmaceutically acceptable salts of the compounds described herein, in contrast to the free base of the respective compounds. In some embodiments, the pharmaceutically acceptable salt is the hydrochloride. For example, FIG. 1f shows the hydrochloride salts of the corresponding free base compounds in FIG. 1e.

Also included are pharmaceutically acceptable solvates. As used herein, the term “solvate” means a compound of the present invention or a salt thereof, that further includes a stoichiometric or non-stoichiometric amount of solvent, e.g., water or organic solvent, bound by non-covalent intermolecular forces.

As used herein, treatment means any manner in which one or more of the symptoms of a disease or disorder are ameliorated or otherwise beneficially altered. Treatment also encompasses any pharmaceutical use of the compositions herein, such as use for treating diseases or disorders in which α-synuclein fibril formation is implicated.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, IC₅₀ refers to an amount, concentration or dosage of a particular test compound that achieves a 50% inhibition of a maximal response, such as modulation of α-synuclein fibril formation, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. In the case of amino acid residues, such residues may be of either the L- or D-form. The configuration for naturally occurring amino acid residues is generally L. When not specified the residue is the L form. As used herein, the term “amino acid” refers to α-amino acids which are racemic, or of either the D- or L-configuration. The designation “d” preceding an amino acid designation (e.g., dAla, dSer, dVal, etc.) refers to the D-isomer of the amino acid. The designation “dl” preceding an amino acid designation (e.g., dlPip) refers to a mixture of the L- and D-isomers of the amino acid. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, “alkyl,” “alkenyl” and “alkynyl” carbon chains, if not specified, contain from 1 to 20 carbons, or 1 or 2 to 16 carbons, and are straight or branched. Alkenyl carbon chains of from 2 to 20 carbons, in certain embodiments, contain 1 to 8 double bonds and alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, allyl (propenyl) and propargyl (propynyl). As used herein, lower alkyl, lower alkenyl, and lower alkynyl refer to carbon chains having from about 1 or about 2 carbons up to about 6 carbons. As used herein, “alk(en)(yn)yl” refers to an alkyl group containing at least one double bond and at least one triple bond.

As used herein, “cycloalkyl” refers to a saturated mono- or multi-cyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion. “Cycloalk(en)(yn)yl” refers to a cycloalkyl group containing at least one double bond and at least one triple bond.

As used herein, “aryl” refers to optionally substituted aromatic monocyclic or multicyclic groups containing from 6 to 19 carbon atoms. Examples of “aryl” groups include phenyl, biphenyl, and the like. Aryl groups also include fused polycyclic aromatic ring systems such as naphthyl, tetrahydronapthyl, pyrenyl, anthracyl, 9,10-dihydroanthracyl, fluorenyl, indenyl, indanyl, and the like, in which a carbocyclic aromatic ring is fused to one or more other aryl, cycloalkyl, or cycloaliphatic rings.

As used herein, “heteroaryl” refers to an optionally substituted monocyclic or multicyclic aromatic ring system, in certain embodiments, of about 5 to about 15 members where one or more, in various embodiments 1 to 3, of the atoms in the ring system is a heteroatom, including but not limited to, nitrogen, oxygen or sulfur. The heteroaryl group may be optionally fused to a benzene ring. Examples of heteroaryl groups include optionally substituted pyridyl, pyrimidyl, pyrazinyl, triazinyl, pyranyl, pyrrolyl, imidazolyl, pyrazolyl, 1,2,3-triazolyl, 1,2,4-triazolyl, tetrazolyl, thienyl, thiazoyl, isothiazolyl, furanyl, oxazolyl, isooxazolyl, and the like. Heteroaryl groups also include fused polycyclic aromatic ring systems in which a heteroaryl ring is fused to one or more other heteroaryl, aryl, heterocyclyl, cycloalkyl, or cycloaliphatic rings, for example, optionally substituted quinolinyl, isoquinolinyl, quinazolinyl, napthyridyl, pyridopyrimidyl, benzothienyl, benzothiazolyl, benzoisothiazolyl, thienopyridyl, thiazolopyridyl, isothiazolopyridyl, benzofuranyl, benzooxazolyl, benzoisooxazolyl, furanopyridyl, oxazolopyridyl, isooxazolopyridyl, indolyl, isoindolyl, benzimidazolyl, benzopyrazolyl, pyrrolopyridyl, isopyrrolopyridyl, imidazopyridyl, pyrazolopyridyl, and the like.

Any ring recited as a substituent herein can be bonded via any substitutable atom in the ring.

As used herein, a “heteroarylium” group is a heteroaryl group that is positively charged on one or more of the heteroatoms.

As used herein, “heterocyclyl” refers to an optionally substituted monocyclic or multicyclic non-aromatic ring system, in various embodiments of 3 to 10 members, in another embodiment of 4 to 7 members, in a further embodiment of 5 to 6 members, where one or more, in certain embodiments, 1 to 3, of the atoms in the ring system is a heteroatom, including but not limited to, nitrogen, oxygen or sulfur. Examples of heterocyclyl groups include oxazolinyl, thiazolinyl, oxazolidinyl, thiazolidinyl, tetrahydrofuranyl, tetrahyrothiophenyl, morpholino, thiomorpholino, pyrrolidinyl, piperazinyl, piperidinyl, thiazolidinyl, and the like. In embodiments where the heteroatom(s) is (are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen may be quaternized to form an ammonium group where the substituents are selected as above.

As used herein. “lone pair,” when referring to a substitution variable on a nitrogen atom, means that the substitution variable represents the Lewis structure electron pair for the corresponding nitrogen, and no substituting functional group is bound to the indicated position.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyanide, cyanate, thiocyanate, selenocyanate, trifluoromethoxy, and azide.

As used herein, “haloalkyl” refers to an alkyl group in which one or more of the hydrogen atoms are replaced by halogen. Such groups include, but are not limited to, chloromethyl, trifluoromethyl and 1-chloro-2-fluoroethyl.

As used herein, “haloalkoxy” refers to RO— in which R is a haloalkyl group.

As used herein, “sulfinyl” or “thionyl” refers to —S(O)—. As used herein, “sulfonyl” or “sulfuryl” refers to —S(O)₂—. As used herein, “sulfo” refers to —S(O)₂O—.

As used herein, “carboxy” refers to a divalent radical, —C(O)O—.

As used herein, “aminocarbonyl” refers to —C(O)NH₂.

As used herein, “alkylaminocarbonyl” refers to —C(O)NHR in which R is alkyl, including lower alkyl. As used herein, “dialkylaminocarbonyl” refers to —C(O)NR′R in which R′ and R are independently alkyl, including lower alkyl; “carboxamide” refers to groups of formula —NR′COR in which R′ and R are independently alkyl, including lower alkyl.

As used herein, “diarylaminocarbonyl” refers to —C(O)NRR′ in which R and R′ are independently selected from aryl, including lower aryl, such as phenyl.

As used herein, “arylalkylaminocarbonyl” refers to —C(O)NRR′ in which one of R and R′ is aryl, including lower aryl, such as phenyl, and the other of R and R′ is alkyl, including lower alkyl.

As used herein, “arylaminocarbonyl” refers to —C(O)NHR in which R is aryl, including lower aryl, such as phenyl.

As used herein, “hydroxycarbonyl” refers to —COOH.

As used herein, “alkoxycarbonyl” refers to —C(O)OR in which R is alkyl, including lower alkyl.

As used herein, “aryloxycarbonyl” refers to —C(O)OR in which R is aryl, including lower aryl, such as phenyl.

As used herein, “alkoxy” and “alkylthio” refer to RO— and RS—, in which R is alkyl, including lower alkyl.

As used herein, “aryloxy” and “arylthio” refer to RO— and RS—, in which R is aryl, including lower aryl, such as phenyl.

As used herein, “alkylene” refers to a straight, branched or cyclic, in certain embodiments straight or branched, divalent aliphatic hydrocarbon group, in various embodiments having from 1 to about 20 carbon atoms, in another embodiment having from 1 to 12 carbons. In a further embodiment alkylene includes lower alkylene. There may be optionally inserted along the alkylene group one or more oxygen, sulfur, including S(═O) and S(═O)₂ groups, or substituted or unsubstituted nitrogen atoms, including —NR— and —N⁺RR— groups, where the nitrogen substituent(s) is (are) alkyl, aryl, aralkyl, heteroaryl, heteroaralkyl or COR′, where R′ is alkyl, aryl, aralkyl, heteroaryl, heteroaralkyl, —OY or —NYY, where Y is hydrogen, alkyl, aryl, heteroaryl, cycloalkyl or heterocyclyl. Alkylene groups include, but are not limited to, methylene (—CH₂—), ethylene (—CH₂CH₂—), propylene (—(CH₂)₃—), methylenedioxy (—O—CH₂—O—) and ethylenedioxy (—O—(CH₂)₂—O—). The term “lower alkylene” refers to alkylene groups having 1 to 6 carbons. In certain embodiments, alkylene groups are lower alkylene, including alkylene of 1 to 3 carbon atoms.

As used herein, “azaalkylene” refers to —(CRR)ₙ—NR—(CRR)ₘ—, where n and m are each independently an integer from 0 to 4. As used herein, “oxaalkylene” refers to —(CRR)ₙ—O—(CRR)ₘ—, where n and m are each independently an integer from 0 to 4. As used herein, “thiaalkylene” refers to —(CRR)ₙ—S—(CRR)ₘ—, —(CRR)ₙ—S(═O)—(CRR)ₘ—, and —(CRR)ₙ—S(═O)₂—(CRR)ₘ—, where n and m are each independently an integer from 0 to 4.

As used herein, “alkenylene” refers to a straight, branched or cyclic, in various embodiments straight or branched, divalent aliphatic hydrocarbon group, in certain embodiments having from 2 to about 20 carbon atoms and at least one double bond, in other embodiments 1 to 12 carbons. In further embodiments, alkenylene groups include lower alkenylene. There may be optionally inserted along the alkenylene group one or more oxygen, sulfur or substituted or unsubstituted nitrogen atoms, where the nitrogen substituent is alkyl. Alkenylene groups include, but are not limited to, —CH═CH—CH═CH— and —CH═CH—CH₂—. The term “lower alkenylene” refers to alkenylene groups having 2 to 6 carbons. In certain embodiments, alkenylene groups are lower alkenylene, including alkenylene of 3 to 4 carbon atoms.

As used herein, “alkynylene” refers to a straight, branched or cyclic, in certain embodiments straight or branched, divalent aliphatic hydrocarbon group, in various embodiments having from 2 to about 20 carbon atoms and at least one triple bond, in another embodiment 1 to 12 carbons. In a further embodiment, alkynylene includes lower alkynylene. There may be optionally inserted along the alkynylene group one or more oxygen, sulfur or substituted or unsubstituted nitrogen atoms, where the nitrogen substituent is alkyl. Alkynylene groups include, but are not limited to, —C≡C—C≡C—, —C≡C— and —C≡C—CH₂—. The term “lower alkynylene” refers to alkynylene groups having 2 to 6 carbons. In certain embodiments, alkynylene groups are lower alkynylene, including alkynylene of 3 to 4 carbon atoms.

As used herein, “alk(en)(yn)ylene” refers to a straight, branched or cyclic, in certain embodiments straight or branched, divalent aliphatic hydrocarbon group, in various embodiments having from 2 to about 20 carbon atoms and at least one triple bond, and at least one double bond; in another embodiment 1 to 12 carbons. In further embodiments, alk(en)(yn)ylene includes lower alk(en)(yn)ylene. There may be optionally inserted along the alkynylene group one or more oxygen, sulfur or substituted or unsubstituted nitrogen atoms, where the nitrogen substituent is alkyl. Alk(en)(yn)ylene groups include, but are not limited to, —C═C—(CH₂)ₙ—C≡C— where n is 1 or 2. The term “lower alk(en)(yn)ylene” refers to alk(en)(yn)ylene groups having up to 6 carbons. In certain embodiments, alk(en)(yn)ylene groups have about 4 carbon atoms.

As used herein, “cycloalkylene” refers to a divalent saturated mono- or multicyclic ring system, in certain embodiments of 3 to 10 carbon atoms, in other embodiments 3 to 6 carbon atoms; cycloalkenylene and cycloalkynylene refer to divalent mono- or multicyclic ring systems that respectively include at least one double bond and at least one triple bond. Cycloalkenylene and cycloalkynylene groups may, in certain embodiments, contain 3 to 10 carbon atoms, with cycloalkenylene groups in certain embodiments containing 4 to 7 carbon atoms and cycloalkynylene groups in certain embodiments containing 8 to 10 carbon atoms. The ring systems of the cycloalkylene, cycloalkenylene and cycloalkynylene groups may be composed of one ring or two or more rings which may be joined together in a fused, bridged or spiro-connected fashion. “Cycloalk(en)(yn)ylene” refers to a cycloalkylene group containing at least one double bond and at least one triple bond.

As used herein, “arylene” refers to a monocyclic or polycyclic, in certain embodiments monocyclic, divalent aromatic group, in various embodiments having from 5 to about 20 carbon atoms and at least one aromatic ring, in another embodiment 5 to 12 carbons. In further embodiments, arylene includes lower arylene. Arylene groups include, but are not limited to, 1,2-, 1,3- and 1,4-phenylene. The term “lower arylene” refers to arylene groups having 6 carbons.

As used herein, “heteroarylene” refers to a divalent monocyclic or multicyclic aromatic ring system, in various embodiments of about 5 to about 15 atoms in the ring(s), where one or more, in certain embodiments 1 to 3, of the atoms in the ring system is a heteroatom, that is, an element other than carbon, including but not limited to, nitrogen, oxygen or sulfur. The term “lower heteroarylene” refers to heteroarylene groups having 5 or 6 atoms in the ring.

As used herein, “heterocyclylene” refers to a divalent monocyclic or multicyclic non-aromatic ring system, in certain embodiments of 3 to 10 members, in various embodiments 4 to 7 members, in another embodiment 5 to 6 members, where one or more, including 1 to 3, of the atoms in the ring system is a heteroatom, that is, an element other than carbon, including but not limited to, nitrogen, oxygen or sulfur.

As used herein, “substituted alkyl,” “substituted alkenyl,” “substituted alkynyl,” “substituted cycloalkyl,” “substituted cycloalkenyl,” “substituted cycloalkynyl,” “substituted aryl,” “substituted heteroaryl,” “substituted heterocyclyl,” “substituted alkylene,” “substituted alkenylene,” “substituted alkynylene,” “substituted cycloalkylene,” “substituted cycloalkenylene,” “substituted cycloalkynylene,” “substituted arylene,” “substituted heteroarylene” and “substituted heterocyclylene” refer to alkyl, alkenyl, alkynyl, cycloalkyl, cycloalkenyl, cycloalkynyl, aryl, heteroaryl, heterocyclyl, alkylene, alkenylene, alkynylene, cycloalkylene, cycloalkenylene, cycloalkynylene, arylene, heteroarylene and heterocyclylene groups, respectively, that are substituted with one or more substituents, in certain embodiments one, two, three or four substituents, where the substituents are as defined herein. “Optionally substituted”

Suitable optional substituents for a substitutable atom any of the preceding groups, e.g., alkyl, cycloalkyl, aliphatic, cycloaliphatic, alkylene, alkenylene, alkynylene, heteroalkylene, heteroalkenylene, heteroalkynylene, heterocyclic, aryl, and heteroaryl groups, are those substituents that do not substantially interfere with the pharmaceutical activity of the disclosed compounds. A “substitutable atom” is an atom that has one or more valences or charges available to form one or more corresponding covalent or ionic bonds with a substituent. For example, a carbon atom with one valence available (e.g., —C(—H)═) can form a single bond to an alkyl group (e.g., —C(-alkyl)=), a carbon atom with two valences available (e.g., —C(H₂)—) can form one or two single bonds to one or two substituents (e.g., —C(alkyl)(H)—, —C(alkyl)(Br))—,) or a double bond to one substituent (e.g., —C(═O)—), and the like. Substitutions contemplated herein include only those substitutions that form stable compounds.

For example, suitable optional substituents for substitutable carbon atoms include —F, —Cl, —Br, —I, —CN, —NO₂, —OR^{a}, —C(O)R^{a}, —OC(O)R^{a}, —C(O)OR^{a}, —SR^{a}, —C(S)R^{a}, —OC(S)R^{a}, —C(S)OR^{a}, —C(O)SR^{a}, —C(S)SR^{a}, —S(O)R^{a}, —SO₂R^{a}, —SO₃R^{a}, —OSO₂R^{a}, —OSO₃R^{a}, —PO₂R^{a}R^{b}, —OPO₂R^{a}R^{b}, —PO₃R^{a}R^{b}, —OPO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), —NR^{a}N(R^{a}R^{b}), —CR^{c}═CR^{a}R^{b}, —C≡CR^{a}, ═O, ═S, ═CR^{a}R^{b}, ═NR^{a}, ═NOR^{a}, ═NNR^{a}, optionally substituted alkyl, optionally substituted cycloalkyl, optionally substituted aliphatic, optionally substituted cycloaliphatic, optionally substituted heterocyclic, optionally substituted benzyl, optionally substituted aryl, and optionally substituted heteroaryl, wherein R^{a}-R^{d} are each independently —H or an optionally substituted aliphatic, optionally substituted cycloaliphatic, optionally substituted heterocyclic, optionally substituted benzyl, optionally substituted aryl, or optionally substituted heteroaryl, or, —N(R^{a}R^{b}), taken together, is an optionally substituted heterocyclic group.

Suitable substituents for nitrogen atoms having two covalent bonds to other atoms include, for example, optionally substituted alkyl, optionally substituted cycloalkyl, optionally substituted aliphatic, optionally substituted cycloaliphatic, optionally substituted heterocyclic, optionally substituted benzyl, optionally substituted aryl, optionally substituted heteroaryl, —CN, —NO₂, —OR^{a}, —C(O)R^{a}, —OC(O)R^{a}, —C(O)OR^{a}, —SR^{a}, —S(O)R^{a}, —SO₂R^{a}, —SO₃R^{a}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)N(R^{a}), —NR^{c}(O)OR^{a}, —NR^{c}C(O)N(R^{a}R^{b}), and the like.

A nitrogen-containing group, for example, a heteroaryl or non-aromatic heterocycle, can be substituted with oxygen to form an N-oxide, e.g., as in a pyridyl N-oxide, piperidyl N-oxide, and the like. For example, in various embodiments, a ring nitrogen atom in a nitrogen-containing heterocyclic or heteroaryl group can be substituted to form an N-oxide.

Suitable substituents for nitrogen atoms having three covalent bonds to other atoms include —OH, alkyl, and alkoxy (preferably C₁₋₆ alkyl and alkoxy). Substituted ring nitrogen atoms that have three covalent bonds to other ring atoms are positively charged, which is balanced by counteranions corresponding to those found in pharmaceutically acceptable salts, such as chloride, bromide, fluoride, iodide, formate, acetate and the like. Examples of other suitable counteranions are provided in the section below directed to suitable pharmacologically acceptable salts.

It will also be understood that certain disclosed compounds can be obtained as different stereoisomers diastereomers and enantiomers) and that the invention includes all isomeric forms and racemic mixtures of the disclosed compounds and methods of treating a subject with both pure isomers and mixtures thereof, including racemic mixtures. Stereoisomers can be separated and isolated using any suitable method, such as chromatography.

It will also be understood that certain disclosed compounds can exist as or can be represented as tautomers. Tautomers are compounds that can be interconverted by migration of a hydrogen atom or proton in combination with the exchange of adjacent single bond and double bonds. In solutions where tautomerization is possible, a chemical equilibrium of the tautomers can be reached. The exact ratio of the tautomers depends on several factors, including temperature, solvent, and pH.

As used herein, “alkylidene” refers to a divalent group, such as ═CR′R″, which is attached to one atom of another group, forming a double bond. Alkylidene groups include, but are not limited to, methylidene (═CH₂) and ethylidene (═CHCH₃). As used herein, “arylalkylidene” refers to an alkylidene group in which either R′ or R″ is an aryl group. “Cycloalkylidene” groups are those where R′ and R″ are linked to form a carbocyclic ring. “Heterocyclylid-ene” groups are those where at least one of R′ and R″ contain a heteroatom in the chain, and R′ and R″ are linked to form a heterocyclic ring.

As used herein, “amido” refers to the divalent group —C(O)NH—. “Thioamido” refers to the divalent group —C(S)NH—. “Oxyamido” refers to the divalent group —OC(O)NH—. “Thiaamido” refers to the divalent group —SC(O)NH—. “Dithiaamido” refers to the divalent group —SC(S)NH—. “Ureido” refers to the divalent group —HNC(O)NH—. “Thioureido” refers to the divalent group —HNC(S)NH—.

As used herein, “semicarbazide” refers to —NHC(O)NHNH—. “Carbazate” refers to the divalent group —OC(O)NHNH—. “Isothiocarbazate” refers to the divalent group —SC(O)NHNH—. “Thiocarbazate” refers to the divalent group —OC(S)NHNH—. “Sulfonylhydrazide” refers to the divalent group —SO₂NHNH—. “Hydrazide” refers to the divalent group —C(O)NHNH—. “Azo” refers to the divalent group —N═N—. “Hydrazinyl” refers to the divalent group —NH—NH—.

Where the number of any given substituent is not specified (e.g., haloalkyl), there may be one or more substituents present. For example, “haloalkyl” may include one or more of the same or different halogens.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

### B. Compounds

The compounds provided herein for use in the compositions and methods provided herein exhibit in vitro and in vivo activity against α-synuclein mediated diseases and disorders. In various embodiments, the compounds treat or ameliorate one or more symptoms associated with α-synuclein toxicity. In various embodiments, the compounds affect aggregation of α-synuclein or fragments thereof. In another embodiment, the compounds do not affect aggregation, but still exert a therapeutic affect on α-synuclein toxicity.

In various embodiments, the compounds for use in the compositions and methods provided herein are according to Formula I:

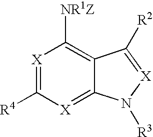

or pharmaceutically acceptable salts or derivatives thereof, wherein:

m is 1 or 2;

n is 0, 1, 2, or 3;

each X is independently N or CH;

R¹ and Z are each independently R⁵, C(O)R⁵, COOR⁵, C(O)NR⁵R⁵, or S(O)ₘR⁵;

R² and R³ are each independently H, halo, pseudohalo, CN, SR⁵, R⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, or NR⁵C(O)C(O)NR⁵R⁶;

R⁴ is independently H; halo, pseudohalo, CN, SR⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, or NR⁵C(O)C(O)NR⁵R⁶; or optionally substituted alkyl, aryl, aralkyl, heteroaryl, or heteroaralkyl; and

each R⁵, R⁶, and R⁸ is independently H or optionally substituted alkyl, alkenyl, alkynyl, cycloalkyl, cycloalkenyl, aryl, heteroaryl, or heterocyclyl.

In various embodiments, R¹ is H.

In various embodiments, the compound is represented by Formula Ib or Ic

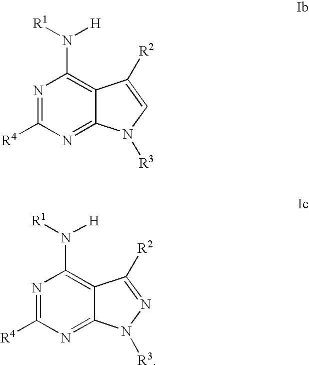

In various embodiments, R² is H, halo, CN, NO₂, NH₂, or C₁-C₁₀ alkyl optionally substituted with 1-3 independent halo, SR⁵, OR⁵, OC(O)R⁵, NR⁵R⁵; COOR⁵, NO₂, CN, C(O)R⁵, OC(O)NR⁵R⁵, or C(O)NR⁵R⁵. In some embodiments, R² is H, F, Cl, Br, CF₃, CCl₃, CN, NO₂, NH₂, or C₁-C₆ alkyl. In some embodiments, R² is aryl, heteroaryl, aralkyl, or heteroaralkyl, each substituted with: H, halo, SR⁵, OR⁵, OC(O)R⁵, NR⁵R⁵; COOR⁵, NO₂, CN, C(O)R⁵, OC(O)NR⁵R⁵, or C(O)NR⁵R⁵; or aryl, C₁-C₁₀ alkyl, or C₂-C₁₀ alkenyl each optionally substituted with 1-3 independent aryl, halo, SR⁵, OR⁵, OC(O)R⁵, NR⁵R⁵; COOR⁵, NO₂, CN, C(O)R⁵, OC(O)NR⁵R⁵, or C(O)NR⁵R⁵. The optionally substituted aryl, heteroaryl, aralkyl, or heteroaralkyl groups in R² may be as described in the Detailed Description, or may be selected, for example, from phenyl, napthyl, benzyl, phenylethylene, napthylmethylene, phenoxymethylene, napthyloxymethylene, pyridylmethylene, benzofurylmethylene, dihydrobenzofurylmethylene, benzodioxolmethylene, indanylmethylene, furyl, thienyl, pyridyl, benzothienyl, and benzofuryl. The optional substituents for the aryl, heteroaryl, aralkyl, or heteroaralkyl groups in R² may be as described in the Detailed Description, or in some embodiments may be selected from: H, F, Cl, Br, OH, C₁-C₆ alkoxy, amino, C₁-C₆ alkylamino, COOH, COO—C₁-C₆ alkyl, NO₂, CN, or C(O)—C₁-C₆ alkyl; or C₁-C₆ alkyl, C₂-C₆ alkenyl, or aryl optionally substituted with phenyl, F, Cl, Br, C₁-C₆ alkoxy, COOH, COO—C₁-C₆ alkyl, NO₂, or CN.

In various embodiments, R² is phenyl, napthyl, benzofuryl, benzothienyl, furyl, or thienyl, each optionally substituted with: halo, CN, amino, alkylamino, C₁-C₆ hydroxyalkyl, S—C₁-C₆ alkyl, C₁-C₆ alkoxy, C₁-C₆ haloalkoxy, COOH, COO—C₁-C₆ alkyl, C(O)—C₁-C₆ alkyl, or C₃-C₆ cycloalkyl; or optionally halogenated aryl, aralkyl, O-aryl, or O-aralkyl. In some embodiments, R² is optionally substituted phenyl, napthyl, benzofuryl, benzothienyl, furyl, thienyl, fluoronapthyl, benzyloxyphenyl, (chlorobenzyl)oxyphenyl, hydroxymethylphenyl, cyclohexylphenyl, chorophenyl, cyanophenyl, carboxyl phenyl, alkyl carboxyl phenyl, alkanoyl phenyl, alkylamino phenyl, trifluoromethoxyphenyl, alkoxyphenyl, phenoxyphenyl, biphenyl, or alkyl-S-phenyl. In some embodiments, R² is aralkyl, aralkenyl, or heteroaralkyl, each optionally substituted with halo, CN, amino, alkylamino, S—C₁-C₆ alkyl, C₁-C₆ alkoxy, C₁-C₆ haloalkoxy, C₁-C₆ haloalkyl, C₂-C₆ alkynyl, aryl, haloaryl, or heteroaryl. In some embodiments, R² is CH₂, CH(CH₃), CH═CH, or CH₂CH₂, each substituted with phenyl, naphthyl, tetrahydronaphthyl, pyridyl, indanyl, benzofuryl, benzodioxolyl, dihydrobenzofuranyl, or tetrahydronaphthyl, wherein each phenyl, napthyl, tetrahydronaphthyl, pyridyl, indanyl, benzofuryl, benzodioxolyl, dihydrobenzofuranyl, or tetrahydronaphthyl in R² is optionally substituted with one or two substituents selected from the group consisting of F, Cl, CF₃; C₁-C₆ alkyl, C₁-C₆ alkoxy, acetylenyl, CN, alkylamino, and phenyl. In certain embodiments, R² is CH(CH₃)-phenyl, CH═CH-phenyl, CH₂CH₂-phenyl, CH₂-naphthyl, CH₂-(methylnaphthyl), CH₂-(fluoronaphthyl), CH₂-pyridyl, CH₂-indanyl, CH₂-benzofuryl, CH₂-benzodioxolyl, CH₂-dihydrobenzofuranyl, CH₂-tetrahydronaphthyl, dichlorobenzyl, (chloro,trifluoromethyl)benzyl, (fluoro,trifluoromethyl)benzyl, (fluoro,chloro)benzyl, dimethylbenzyl, (methyl,fluoro)benzyl, dimethoxybenzyl, (acetylenyl)benzyl, cyanobenzyl, (dimethylamino)benzyl, methoxybenzyl, or phenylbenzyl.

In various embodiments, R³ is H; C₁-C₁₀ alkyl or C₂-C₁₀ alkenyl each optionally substituted with 1-3 halo, CF₃, SR⁵, OR⁵, OC(O)R⁵, NR⁵R⁵; COOR⁵, NO₂, CN, C(O)R⁵, OC(O)NR⁵R⁵, C(O)NR⁵R⁵; C₃-C₁₀ cycloalkyl; or C₂-C₁₀ alkynyl. In some embodiments, R³ is H, C₁-C₈ alkyl optionally substituted with 1-3 halo, OR⁵, NR⁵R⁵, COOR⁵, C(O)R⁵, C(O)NR⁵R⁵, C₂-C₆ alkenyl, or C₂-C₆ alkynyl; or cyclopropyl, cyclopropylmethyl, cyclobutyl, cyclobutylmethyl, cyclopentyl, cyclopentylmethyl, cyclohexyl, or cyclohexylmethyl. In certain embodiments, R³ is aryl, heteroaryl, aralkyl, heteroaralkyl, heterocyclyl, or heterocyclyalkyl, each substituted with: H, alkyl, halo, OR⁵, OC(O)R⁵, NR⁵R⁵; COOR⁵, NO₂, CN, C(O)R⁵, OC(O)NR⁵R⁵, or C(O)NR⁵R⁵; or optionally substituted aryl, heteroaryl, or heterocyclyl. The aryl, heteroaryl, aralkyl, heteroaralkyl, heterocyclyl, or heterocyclyalkyl groups represented by R³ may be as described in the Detailed Description or can be selected, for example, from benzyl, pyridyl, pyridylmethylene, furyl, thienyl, tetrahydrofuryl, or tetrahydrothienyl. The substituents for the aryl, heteroaryl, aralkyl, heteroaralkyl, heterocyclyl, or heterocyclyalkyl groups represented by R³ may be as described in the Detailed Description, or can be selected from, for example: H, F, Cl, Br, SR⁵, OR⁵, NR⁵R⁵; COOR⁵, NO₂, CN, C(O)R⁵; or C₁-C₆ alkyl, C₂-C₆ alkenyl, or aryl optionally substituted with phenyl, F, Cl, Br, SR⁵, OR⁵, COOR⁵, NO₂, or CN.

In various embodiments, R³ is optionally substituted aryl; C₁-C₁₀ alkyl optionally substituted with aryl or cycloalkyl; C₃-C₁₀ cycloalkyl; C₂-C₁₀ alkenyl, or C₂-C₁₀ alkynyl. In some embodiments, R³ is optionally substituted propenyl, propynyl, benzyl, cyclobutyl, cyclopropylmethyl, 2,2-dimethylpropyl, cyclohexyl, cyclopentyl, cyclopropyl, phenylethylene, ethyl, 2-propyl, methyl, phenyl, nitrophenyl, sec-butyl, or tert-butyl.

In various embodiments, R⁴ is independently aryl; heteroaryl; C₁-C₁₀ alkyl or C₂-C₁₀ alkenyl, each optionally substituted with 1-3 independent aryl, R⁷, or heteroaryl; C₂-C₁₀ alkynyl; halo; haloalkyl; CF₃; SR⁵; OR⁵; OC(O)R⁵; NR⁵R⁵; NR⁵R⁶; COOR⁵; NO₂; CN; C(O)R⁵; C(O)C(O)R⁵; C(O)NR⁵R⁵; S(O)ₘR⁵; S(O)ₘNR⁵R⁵; NR⁵C(O)NR⁵R⁵; NR⁵C(O)C(O)R⁵; NR⁵C(O)R⁵; NR⁵(COOR⁵); NR⁵C(O)R⁸; NR⁵S(O)ₘNR⁵R⁵; NR⁵S(O)ₘR⁵; NR⁵S(O)ₘR⁸; NR⁵C(O)C(O)NR⁵R⁵; or NR⁵C(O)C(O)NR⁵R⁶. In some embodiments, R⁴ is: H; OR⁵; OC(O)R⁵; NR⁵R⁵; COOR^{S}; NO₂; CN; C(O)R⁵; C(O)C(O)R⁵; or C(O)NR⁵R⁵; or C₁-C₁₀ alkyl optionally substituted with 1-3 halo, OR⁵, OC(O)R⁵, NR⁵R⁵; COOR⁵, NO₂, CN, C(O)R⁵, OC(O)NR⁵R⁵, or C(O)NR⁵R⁵. In certain embodiments, R⁴ is an optionally substituted aryl, aralkyl, heteroaryl, or heteroaralkyl, wherein the aryl, aralkyl, heteroaryl, or heteroaralkyl groups may be as described in the Detailed Description or can be selected, for example, from phenyl, benzyl, pyridyl, pyridylmethylene, furyl, furylmethylene, thienyl, thienylmethylene, pyrazolyl, and pyrazolylmethylene. The optional substituents for the optionally substituted aryl, aralkyl, heteroaryl, or heteroaralkyl groups represented by R⁴ may be as described in the Detailed Description, or can be selected from, for example: H, CF₃, CCl₃, amino, C₁-C₆ alkoxy, COOH, COO—C₁-C₆ alkyl, OC(O)—C₁-C₆ alkyl, phenoxy, or alkylphenoxy; or C₁-C₆ alkyl optionally substituted with amino, COOH, COO—C₁-C₆ alkyl or OC(O)—C₁-C₆ alkyl, or 1 or 2 C₁-C₆ alkoxy. In some embodiments, the optional substituents are halo, CF₃, SR⁵, OR⁵, OC(O)R⁵, NR⁵R⁵; COOR⁵, NO₂, CN, C(O)R⁵, OC(O)NR⁵R⁵, C(O)NR⁵R⁵, N(R⁵)C(O)R⁵, N(R⁵)(COOR⁵), or S(O)ₘNR⁵R⁵. In certain embodiments, the optional substituents are F, Cl, OH, amino, NO₂, C₁-C₆ alkoxy, C₁-C₆ alkyl, phenoxy, or alkylphenoxy; or phenyl, imidazolyl, or morpholino optionally substituted with F, Cl, amino, NO₂, C₁-C₆ alkoxy, or C₁-C₆ alkyl.

In various embodiments, wherein R⁴ is independently amino, alkylamino, or aryl, heteroaryl, or C₁-C₁₀ alkyl optionally substituted with halo, CF₃, O—C₁-C₆ alkyl, or aryloxy. In some embodiments, R⁴ is pyridyl, C₁-C₆ alkoxy-C₁-C₆ alkyl, (C₁-C₆ alkyl)phenoxy-C₁-C₆ alkyl, C₁-C₆ alkyl, amino, or halophenyl. In certain embodiments, R⁴ is pyridyl, CH(OCH₂CH₃)₂, tert-butyl-phenyoxymethylene, methyl, ethyl, amino, or chlorophenyl. In some embodiments, R⁴ is pyridyl or C₁-C₆ alkyl. In some embodiments, R⁴ is pyridyl, methyl, or ethyl.

In various embodiments, the compound is selected from the compounds in FIGS. 1a, 1b, 1c, 1d, 1e, or 1f. In some embodiments, the compound is selected from the compounds in FIGS. 1a, 1b, or 1f. In certain embodiments, the compound is selected from the compounds in FIGS. 1a and 1b, 1b and 1f, 1a and 1f, 1a, 1b, or 1f. In various embodiments, the compounds do not include the compounds of one or more of FIGS. 1c, 1d, and/or 1e; for example, in some embodiments, the compound is not a compound in FIGS. 1c, 1d, or 1e. In some embodiments, the compounds do not include the compounds of one or more of FIGS. 1c, 1d, 1e, and/or 1f.

In various embodiments, when R¹ and Z are H, R² is 5-NO₂-fur-2-yl, or phenyl optionally substituted with a single 4-Cl, 4-CH₃, or 4-OCH₃; and R³ is unsubstituted phenyl, cyclohexyl, or acyclic C₁-C₄ alkyl; and the compound is in the form of a free base; then R⁴ is not H, unsubstituted C₁-C₄ alkyl, or phenyl optionally substituted with 4-Cl or 4-CH₃. In various embodiments, when R¹ and Z are H, R² is CN or CH₂CN; and R³ is CH₃, or phenyl optionally substituted with 4-NO₂; then R⁴ is not CO₂-alkyl or CCl₃. In various embodiments, when R¹ and Z are H, R³ is cyclopentyl, and R⁴ is unsubstituted 4-pyridyl, then R² is not CF₃; CN, Br, Cl, or NO₂. In various embodiments, when R¹ and Z are H, R³ is cyclopentyl, and R⁴ is optionally substituted 4-pyridyl, then R² is not C₁-C₄ alkyl optionally substituted with F. In various embodiments, when R¹ and Z are H, R³ is unsubstituted C₁-C₄ alkyl, cyclopentyl, or phenyl, and R⁴ is unsubstituted pyridyl, then R² is not unsubstituted CH₃, benzyl, or CH₂-pyrid-4-yl, and then R² is not H when the compound is in the form of a free base. In various embodiments, when R¹ and Z are H, R² is H or unsubstituted C₁-C₂ alkyl, benzyl, or CH₂-pyridyl; and R⁴ is unsubstituted 4-pyridyl, then R³ is not a lone pair, C₁-C₄ alkyl optionally substituted with CO₂-alkyl, dialkylamino, or cyclopentyl; benzyl optionally substituted with Cl, CN, or CH₃; unsubstituted cyclobutyl, cyclopentyl, 3-tetrahydrofuryl, or 2-bicyclo[2.2.1]heptyl; and R³ is not H when the compound is in the form of a free base. In various embodiments, when R¹ and Z are H, R³ is H, a lone pair, cyclopentyl, 3-(5-ethyl-5H-[1,2,4]triazino[5,6-b]indolyl); unsubstituted benzyl; C₁-C₄ alkyl optionally substituted with OCH₃; phenyl optionally substituted with Cl, 3-NO₂, 4-NO₂, or 4-Me; or ribofuranose; and R⁴ is 2-furyl optionally substituted with 5-NO₂; 5-NH₂-pyrazol-4-yl optionally substituted with methyl or optionally chlorinated phenyl; phenyl optionally substituted with imidazolyl, 4-Cl, 4-OH, or 4-NO₂; C₁-C₄ alkyl optionally substituted with F or acetate; or unsubstituted benzyl; then R² is not unsubstituted C₁-C₂ alkyl, and when the compound is in the form of a free base, R² is not H. In various embodiments, when R¹ and Z are H, R³ is H or a lone pair, and R⁴ is phenyl optionally substituted with OH, NH₂, NO₂, NHC(O)NHPhSO₂F, NHC(O)PhSO₂F; fur-2-yl with an optional 5-NO₂ group, 3-NH₂-pyrazol-4-yl; C₁-C₄ alkyl optionally substituted with F or CO₂-alkyl; or unsubstituted pyridyl or benzyl; then R² is not CN, and R² is not H when the compound is in the form of a free base. In various embodiments, when R³ is tert-butyl; R⁴ is H; R¹ and Z are both H or acetyl, or R¹ is H and Z is acetyl, optionally substituted SO₂-phenyl, or substituted benzoyl; then R² is not H or Br; phenyl optionally 3 or 4-substituted with OCH₃, phenoxy or benzyloxy, or substituted only with a single Cl, 4-CF₃, 4-F, 4-C₁-C₄ alkyl, or 4-phenyl; benzyl optionally substituted with Cl, F, or CH₃; unsubstituted naphthyl, CH₂-naphthyl, or OCH₂-naphthyl; or unsubstituted thien-2-yl or benzothien-2-yl.

In some embodiments, when R¹ and Z are H, R² is nitrofuryl, or phenyl optionally substituted with halo, alkyl, or alkoxy; and R³ is unsubstituted alkyl, cycloalkyl, or phenyl; then R⁴ is not H, unsubstituted alkyl, or phenyl optionally substituted with Cl or alkyl. In some embodiments, when R¹ and Z are H, R² is CN or CH₂CN; and R³ is alkyl, or phenyl optionally substituted with NO₂; then R⁴ is not CO₂-alkyl or CCl₃. In some embodiments, when R¹ and Z are H, R³ is cycloalkyl, and R⁴ is optionally substituted pyridyl, then R² is not CF₃; CN, Br, Cl, or NO₂, or alkyl optionally substituted with F. In some embodiments, when R¹ and Z are H, R³ is unsubstituted alkyl, cycloalkyl, or phenyl, and R⁴ is unsubstituted pyridyl, then R² is not H or unsubstituted alkyl, benzyl, or CH₂-pyridyl. In some embodiments, when R¹ and Z are H, R² is H or unsubstituted alkyl, benzyl, or CH₂-pyridyl; and R⁴ is unsubstituted pyridyl, then R³ is not H, a lone pair, alkyl optionally substituted with CO₂-alkyl, dialkylamino, or cycloalkyl; benzyl optionally substituted with Cl, CN, or alkyl; unsubstituted cycloalkyl, bicycloalkyl, or tetrahydrofuryl. In some embodiments, when R¹ and Z are H, R² is H or unsubstituted alkyl, and R³ is H, a lone pair, cycloalkyl, a tricyclic heteroaryl substituted with alkyl; unsubstituted benzyl; C₁-C₄ alkyl optionally substituted with OCH₃; phenyl optionally substituted with C₁, NO₂, or Me; or ribofuranose; then R⁴ is not furyl optionally substituted with NO₂; NH₂-pyrazolyl optionally substituted with methyl or optionally chlorinated phenyl; phenyl optionally substituted with imidazolyl, Cl, OH, or NO₂; C₁-C₄ alkyl optionally substituted with F or acetate; or unsubstituted benzyl. In some embodiments, when R¹ and Z are H, R³ is H or a lone pair, and R² is H or CN, then R⁴ is not phenyl optionally substituted with OH, NH₂, NO₂, NHC(O)NHPhSO₂F, NHC(O)PhSO₂F; furyl optionally substituted with NO₂, NH₂-pyrazolyl; C₁-C₄ alkyl optionally substituted with F or CO₂-alkyl; or unsubstituted pyridyl or benzyl. In some embodiments, when R¹ and Z are both H or acetyl, or R¹ is H and Z is acetyl, SO₂-phenyl, or optionally substituted benzoyl, R³ is tert-butyl, and R⁴ is H, then R² is not H or Br; phenyl optionally substituted with Cl, CF₃, F, C₁-C₄ alkyl, phenyl, or OCH₃, phenoxy or benzyloxy; benzyl optionally substituted with Cl, F, or CH₃; unsubstituted naphthyl, CH₂-naphthyl, or OCH₂-naphthyl; or unsubstituted thienyl or benzothienyl.

In certain embodiments, when R¹ and Z are H, R² is nitrofuryl or optionally substituted phenyl; and R³ is unsubstituted alkyl, cycloalkyl, or phenyl; then R⁴ is not H, unsubstituted alkyl, or optionally substituted phenyl. In certain embodiments, when R¹ and Z are H, R² is CN or CH₂CN; and R³ is alkyl, or phenyl optionally substituted with NO₂; then R⁴ is not CO₂-alkyl or CCl₃. In certain embodiments, when R¹ and Z are H, R³ is unsubstituted alkyl, cycloalkyl, or phenyl, and R⁴ is optionally substituted pyridyl, then R² is not H oCF₃; CN, Br, Cl, NO₂, alkyl, haloalkyl, benzyl, or CH₂-pyridyl. In certain embodiments, when R¹ and Z are H, R² is H or unsubstituted alkyl, benzyl, or CH₂-pyridyl; and R⁴ is unsubstituted pyridyl, then R³ is not H, a lone pair, optionally substituted alkyl, dialkylamino, or cycloalkyl; optionally substituted benzyl; cycloalkyl, bicycloalkyl, or tetrahydrofuryl. In certain embodiments, when R¹ and Z are H, R² is H or alkyl, and R³ is H, a lone pair, cycloalkyl, a tricyclic heteroaryl substituted with alkyl; benzyl; alkyl, alkoxyalkyl; optionally substituted phenyl; or ribofuranose; then R⁴ is not optionally substituted furyl, NH₂-pyrazolyl, phenyl, alkyl or benzyl. In certain embodiments, when R¹ and Z are H, R³ is H or a lone pair, and R² is H or CN, then R⁴ is not an optionally substituted phenyl; furyl, pyrazolyl; alkyl, pyridyl or benzyl. In certain embodiments, when R¹ and Z are both H or acetyl, or R¹ is H and Z is acetyl, SO₂-phenyl, or optionally substituted benzoyl, R³ is tert-butyl, and R⁴ is H, then R² is not H or Br; optionally substituted phenyl, phenoxy, benzyloxy, benzyl, naphthyl, CH₂-naphthyl, OCH₂-naphthyl, thienyl or benzothienyl.

In some embodiments, when R¹ and Z are H, R² is nitrofuryl or optionally substituted phenyl; and R³ is alkyl, cycloalkyl, or phenyl; then R⁴ is not H, alkyl, or optionally substituted phenyl. In some embodiments, when R¹ and Z are H, R² is CN or CH₂CN; and R³ is alkyl or optionally substituted phenyl; then R⁴ is not CO₂-alkyl or CCl₃. In some embodiments, when R¹ and Z are H, R³ is unsubstituted alkyl, cycloalkyl, or phenyl, and R⁴ is optionally substituted pyridyl, then R² is not H, CN, Br, C₁, NO₂, alkyl, haloalkyl, benzyl, or CH₂-pyridyl. In some embodiments, when R¹ and Z are H, R² is H or unsubstituted alkyl, benzyl, or CH₂-pyridyl; and R⁴ is unsubstituted pyridyl, then R³ is not H, a lone pair, dialkylamino, or optionally substituted alkyl, cycloalkyl, bicycloalkyl, benzyl, or tetrahydrofuryl. In some embodiments, when R¹ and Z are H, R² is H or alkyl, and R³ is H, a lone pair, cycloalkyl, a substituted tricyclic heteroaryl, benzyl, alkyl, alkoxyalkyl; optionally substituted phenyl; or a sugar; then R⁴ is not optionally substituted furyl, pyrazolyl, phenyl, alkyl or benzyl. In some embodiments, when R¹ and Z are H, R³ is H or a lone pair, and R² is H or CN, then R⁴ is not an optionally substituted phenyl, furyl, pyrazolyl, alkyl, pyridyl or benzyl. In some embodiments, when R¹ and Z are both H or acetyl, or R¹ is H and Z is acetyl, SO₂-phenyl, or optionally substituted benzoyl, R³ is tert-butyl, and R⁴ is H, then R² is not H or Br; optionally substituted phenyl, phenoxy, benzyloxy, benzyl, naphthyl, CH₂-naphthyl, OCH₂-naphthyl, thienyl or benzothienyl.

In some embodiments, the compound is one of:

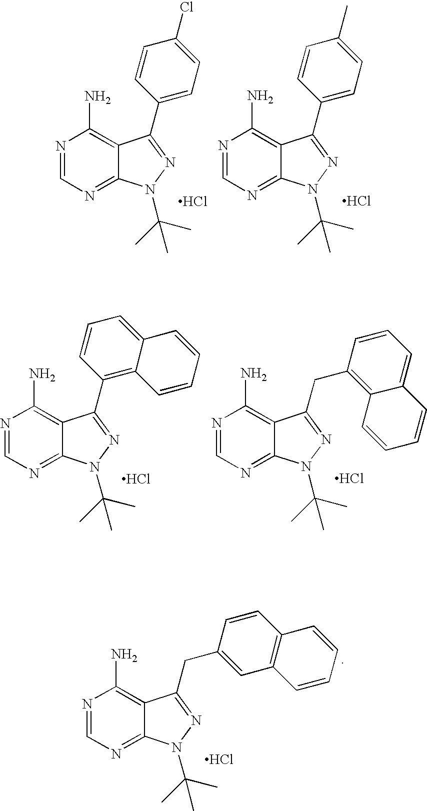

In various embodiments, the compounds for use in the compositions and methods provided herein have a structure according to Formula I:

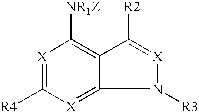

or pharmaceutically acceptable salts or derivatives thereof, where:

n can be 0, 1, 2, or 3;

R² can be H, halo, pseudohalo, (CH₂), —Y, or (CH═CH)ₙ—Y, where Y can be unsubstituted or substituted aryl, heteroaryl, alkyl, or cycloalkyl;

R³ can be substituted or unsubstituted alkyl, alkenyl, alkynyl, aryl, aralkyl, cycloalkyl, (CH₂)ₙ-cycloalkyl, or adamantyl;

R⁴ can be H, NH₂, NR⁵R⁶, NR⁵COR⁶, or unsubstituted or substituted alkyl or aryl;

R¹, Z, R⁵, and R⁶ can be independently selected from H, unsubstituted or substituted alkyl, aralkyl, aryl, alkaryl, or cycloalkyl, COR^{o}^{⁷}, where R^{o}^{⁷} is unsubstituted or substituted alkyl or aryl, SO₂R^{o}^{⁸}, where R^{o}^{⁸} is aryl or substituted aryl, and (CH₂)ₙ-cycloalkyl, where the cycloalkyl may be substituted; and

X can be CH or N.

In some embodiments, possible substituents for Y can be selected from halo, pseudohalo, alkyl, cycloalkyl, aryl, aralkyl, NO₂, alkoxy, aryloxy, arylalkyoxy, CF₃, OCF₃, CN, NR⁵R⁶, NR⁵COR⁶, (CH₂)ₙOR⁶, SR⁶, CO₂H, CO₂R⁶, CONR⁶R⁵, COR⁶, and SO₂NR⁵R⁶.

In some embodiments, possible substituents for R⁴ include halo, alkyl, cycloalkyl, aryl, aralkyl, NO₂, alkoxy, aryloxy, arylalkyoxy, CF₃, OCF₃, CN, NR⁵R⁶, NR⁵COR⁶, (CH₂)ₙOR⁶, SR⁶, CO₂H, CO₂R⁶, CONR⁶R⁵, COR⁶, and SO₂NR⁵R⁶. In some embodiments, substituents for R⁴ groups are halo or alkyl.

In some embodiments, n is 1. In some embodiments, n is 0.

In some embodiments, each X is N.

In some embodiments, R¹ and Z are each independently hydrogen, or substituted or unsubstituted alkyl, arylcarbonyl, aralkylcarbonyl, haloarylcarbonyl, arylsulfonyl, aralkylsulfonyl, or haloarylsulfonyl.

In some embodiments, R¹ and Z are each independently hydrogen, methyl, COR^{o}^{⁷}, where R^{o}^{⁷} is methyl, phenyl, tolyl, 2-chlorophenyl, or 4-fluorophenyl, or SO₂R^{o}^{⁸}, where R^{o}^{⁸} is phenyl, tolyl, or 4-chlorophenyl. In some embodiments, R¹ is H and Z is H. In some embodiments, R¹ is methyl and Z is H.

In some embodiments, R² is hydrogen, halo, or substituted or unsubstituted aryl, heteroaryl, aralkyl, or aralkenyl.

In some embodiments, R² is hydrogen, bromo, phenyl, tolyl, styrenyl, benzyl, naphthyl, naphthyl methyl, 4-biphenyl, 3-methylphenyl, 4-ethylphenyl, 4-isopropylphenyl, 4-(n-butyl)phenyl, 4-tert-butylphenyl, 4-cyclohexylphenyl, 2-methoxyphenyl, 4-methoxyphenyl, 2-chlorophenyl, 3-chlorophenyl, 4-chlorophenyl, 3,4-dichlorophenyl, 4-fluorophenyl, 3,4-difluorophenyl, 4-cyanophenyl, 4-trifluoromethylphenyl, 3-trifluoromethoxyphenyl, 3-methyl-4-fluorophenyl, 4-hydroxymethyl-phenyl, 4-(dimethylamino)phenyl, 4-(ethoxycarbonyl)phenyl, 4-(hydroxycarbonyl)-phenyl, 4-(phenoxy)phenyl, 4-(2-naphtylmethyl)-phenyl, 2-furyl, 3-furyl, 2-thienyl, 3-thienyl, 2-benzofuryl, 4-acetophenone, or 2-benzothienyl.

In some embodiments, R³ is substituted or unsubstituted alkyl, cycloalkyl, aryl, or aralkyl.

In some embodiments, R³ is methyl, ethyl, isopropyl, tert-butyl, 2-dimethylpropyl, 2-propenyl, 2-propynyl, 2-methylbutyl, cyclobutyl, cyclopentyl, cyclohexyl, cyclopropylmethyl, phenyl, or benzyl.

In some embodiments, R⁴ is hydrogen, amino, or substituted or unsubstituted aryl. R⁴ can be hydrogen, amino, tolyl, or 4-chlorophenyl. In some embodiments, R⁴ is H. In some embodiments, R⁴ is amino.

Compounds according to Formula I are also set forth in FIGS. 1a, 1b, 1c, 1d, 1e and 1f, for example, FIGS. 1a, 1b, and 1f, FIGS. 1a and 1b, or FIG. 1a.

### C. Preparation of the Compounds

The compounds for use in the compositions and methods provided herein may be obtained from commercial sources (e.g., Aldrich Chemical Co., Milwaukee, Wis.), may be prepared by methods well known to those of skill in the art, or may be prepared by the methods shown herein, both below and in the Examples. One of skill in the art would be able to prepare all of the compounds for use herein by routine modification of these methods using the appropriate starting materials.

Certain of the compounds provided herein may be made by the synthetic routes shown below. For example, Schemes 1-7 demonstrate a number of methods to perform generic substitution of a pyrazolo-pyrimidine core with various R and Ar groups.

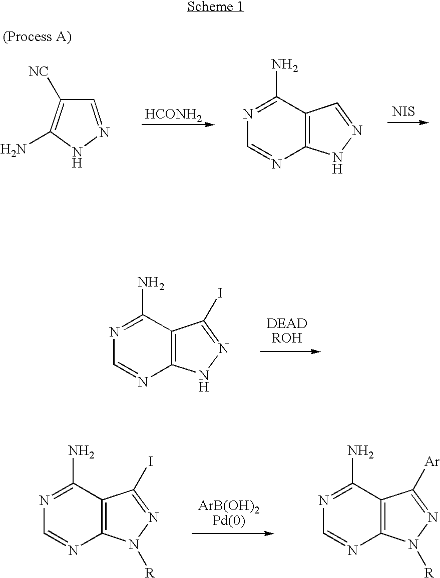

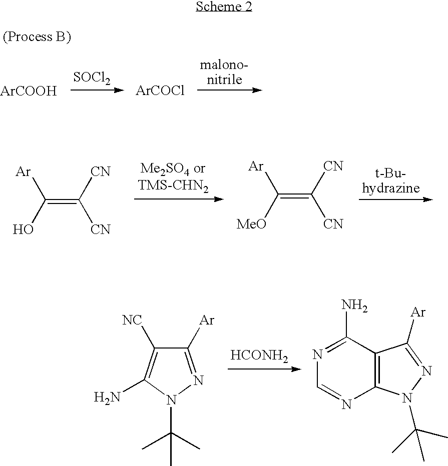

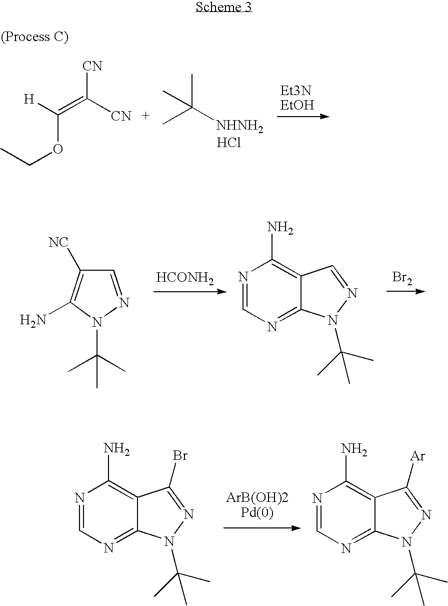

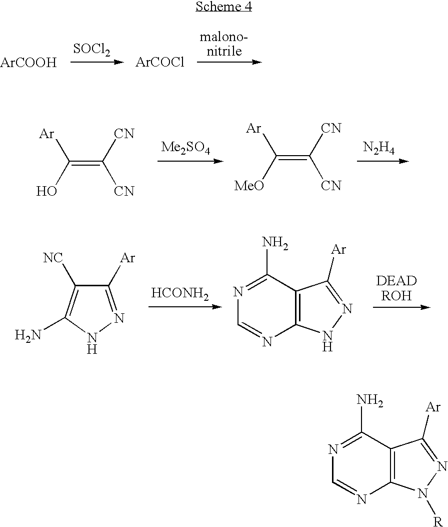

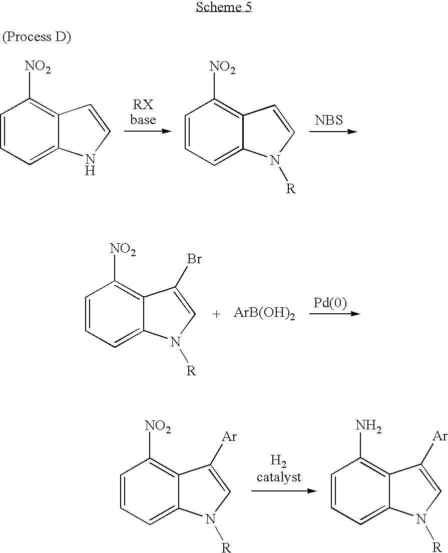

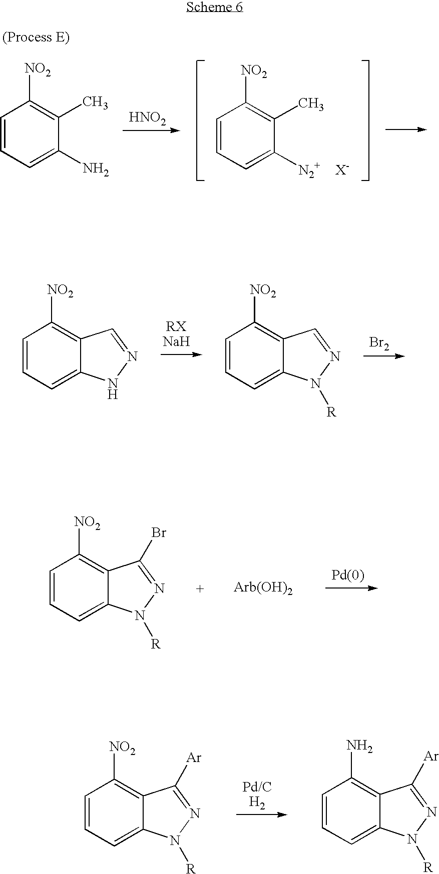

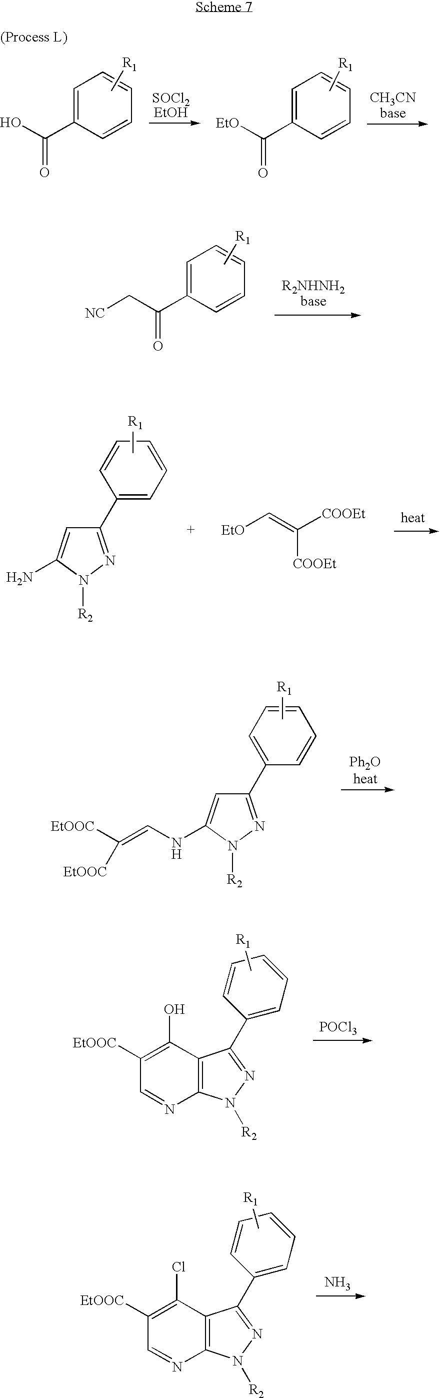

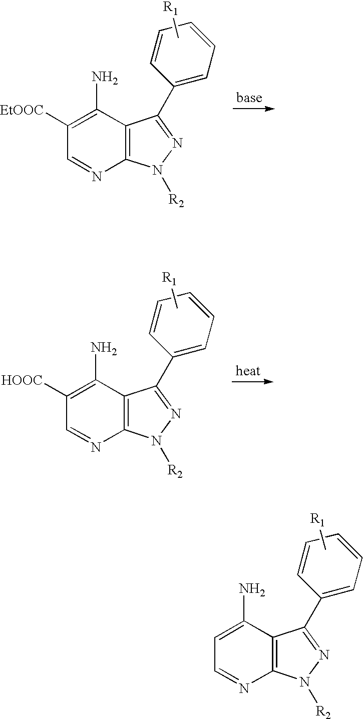

The syntheses of particular compounds prepared by the schemes shown above are also demonstrated in the Examples.

Moreover, synthetic chemistry functional group transformations useful in synthesizing the full range of the disclosed compounds are known in the art and include, for example, those described in R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995). The entire teachings of these documents are incorporated herein by reference. For example, starting with the syntheses above, one can prepare final products having a substituent such as —OH. Suitable techniques for converting the —OH group to another disclosed substituent such as a halogen are well known. For example, an —OH can be converted to —Cl, for example, using a chlorinating reagent such as thionyl chloride or N-chlorosuccinimide, optionally in combination with ultraviolet irradiation.

Suitable protecting groups and strategies for protecting and deprotecting functional groups using protecting groups useful in synthesizing the disclosed compounds are known in the art and include, for example, those described in T. W. Greene and P. G. M. Wuts, Protective Groups in Organic Synthesis, 2nd Ed., John Wiley and Sons (1991), the entire teachings of which are incorporated herein by reference. For example, suitable hydroxyl protecting groups include, but are not limited to substituted methyl ethers (e.g., methoxymethyl, benzyloxymethyl) substituted ethyl ethers (e.g., ethoxymethyl, ethoxyethyl)benzyl ethers (benzyl, nitrobenzyl, halobenzyl) silyl ethers (e.g., trimethylsilyl), esters, and the like. Examples of suitable amine protecting groups include benzyloxycarbonyl, tert-butoxycarbonyl, tert-butyl, benzyl and fluorenylmethyloxy-carbonyl (Fmoc). Examples of suitable thiol protecting groups include benzyl, tert-butyl, acetyl, methoxymethyl and the like.

The reactions described herein may be conducted in any suitable solvent for the reagents and products in a particular reaction. Suitable solvents are those that facilitate the intended reaction but do not react with the reagents or the products of the reaction. Suitable solvents can include, for example: ethereal solvents such as diethyl ether or tetrahydrofuran; ketone solvents such as acetone or methyl ethyl ketone; halogenated solvents such as dichloromethane, chloroform, carbon tetrachloride, or trichloroethane; aromatic solvents such as benzene, toluene, xylene, or pyridine; polar aprotic organic solvents such as acetonitrile, dimethyl sulfoxide, dimethyl formamide, N-methyl pyrrolidone, hexamethyl phosphoramide, nitromethane, nitrobenzene, or the like; polar protic solvents such as methanol, ethanol, propanol, butanol, ethylene glycol, tetraethylene glycol, or the like; nonpolar hydrocarbons such as pentane, hexane, cyclohexane, cyclopentane, heptane, octance, or the like; basic amine solvents such as pyridine, triethyleamine, or the like; and other solvents known to the art.

Reactions or reagents which are water sensitive may be handled under anhydrous conditions. Reactions or reagents which are oxygen sensitive may be handled under an inert atmosphere, such as nitrogen, helium, neon, argon, and the like. Reactions or reagents which are light sensitive may be handled in the dark or with suitably filtered illumination.

Reactions or reagents which are temperature-sensitive, e.g., reagents that are sensitive to high temperature or reactions which are exothermic may be conducted under temperature controlled conditions. For example, reactions that are strongly exothermic may be conducted while being cooled to a reduced temperature.

Reactions that are not strongly exothermic may be conducted at higher temperatures to facilitate the intended reaction, for example, by heating to the reflux, temperature of the reaction solvent. Reactions can also be conducted under microwave irradiation conditions. For example, in various embodiments of the method, the first and second reagents are reacted together under microwave irradiation.

Reactions may also be conducted at atmospheric pressure, reduced pressure compared to atmospheric, or elevated pressure compared to atmospheric pressure. For example, a reduction reaction may be conducted in the presence of an elevated pressure of hydrogen gas in combination with a hydrogenation catalyst.

Reactions may be conducted at stoichiometric ratios of reagents, or where one or more reagents are in excess. For example, in the last step of scheme 3, process C, the first reactant, organohalogen 3-bromo-1-tert-butyl-1H-pyrazolo[3,4-d]pyrimidin-4-amine, may be used in a molar ratio to the aryl boronate reactant represented by ArB(OH)₂ of about 20:1, 10:1, 5:1, 2.5:1, 2:1, 1.5:1, 1.3:1, 1.2:1, 1.1:1, 1:1, 0.91:1, 0.83:1, 0.77:1, 0.67:1, 0.5:1, 0.4:1, 0.2:1, 0.1:1 or 0.5:1. Typically, the first reactant may be used in a molar ratio to the second reactant of about 5:1, 2.5:1, 2:1, 1.5:1, 1.3:1, 1.2:1, 1.1:1, 1:1, 0.91:1, 0.83:1, 0.77:1, 0.67:1, 0.5:1, 0.4:1. In certain embodiments, the first reactant may be used in a molar ratio to the second reactant of about 1.5:1, 1.3:1, 1.2:1, 1.1:1, 1:1, 0.91:1, 0.83:1, 0.77:1, or 0.67:1. Preferably, first reactant may be used in a molar ratio to the second reactant of between about 1.1:1 and 0.9:1, typically about 1:1. The same or different ratios may be used for other reagents in this or other reactions.

### D. Formulation of Pharmaceutical Compositions

The pharmaceutical compositions provided herein contain therapeutically effective amounts of one or more of the compounds provided herein that are useful in the treatment or amelioration of one or more of the symptoms of diseases or disorders associated with α-synuclein toxicity, α-synuclein fibril formation, or in which α-synuclein fibril formation is implicated, and a pharmaceutically acceptable carrier. Diseases or disorders associated with α-synuclein toxicity and/or α-synuclein fibril formation include, but are not limited to, Parkinson's disease and Lewy body dementia. Pharmaceutical carriers suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients.

The compositions contain one or more compounds provided herein. The compounds are, in various embodiments, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In various embodiments, the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives thereof is (are) mixed with a suitable pharmaceutical carrier. The compounds may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions are effective for delivery of an amount, upon administration, that treats or ameliorates one or more of the symptoms of diseases or disorders associated with α-synuclein toxicity, α-synuclein fibril formation or in which α-synuclein toxicity and/or fibril formation is implicated.

In various embodiments, the compositions are formulated for single dosage administration. To formulate a composition, the weight fraction of compound is dissolved, suspended, dispersed or otherwise mixed in a selected carrier at an effective concentration such that the treated condition is relieved or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein (see, e.g., EXAMPLE 1) and in U.S. patent application Ser. No. 10/826,157, filed Apr. 16, 2004, and U.S. Patent Application Publication No. 2003/0073610, and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders associated with α-synuclein fibril formation or in which α-synuclein fibril formation is implicated, as described herein.

In various embodiments, a therapeutically effective dosage should produce a serum concentration of active ingredient of from about 0.1 ng/ml to about 50-100 μg/ml. The pharmaceutical compositions, in another embodiment, should provide a dosage of from about 0.001 mg to about 2000 mg of compound per kilogram of body weight per day. Pharmaceutical dosage unit forms are prepared to provide from about 0.01 mg, 0.1 mg or 1 mg to about 500 mg, 1000 mg or 2000 mg, and in various embodiments from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in various embodiments, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiple's thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, in various embodiments 0.1-95%, in another embodiment 75-85%.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

a. Solid Compositions for Oral Administration

In certain embodiments, the formulations are solid dosage forms, in various embodiments, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or pharmaceutically acceptable derivative thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

b. Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in various embodiments encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

2. Injectables, Solutions and Emulsions

Parenteral administration, in various embodiments characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In various embodiments, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in various embodiments, about neutral pH. Subsequent sterile filtration of the solution followed by lyophilization under standard conditions known to those of skill in the art provides the desired formulation. In various embodiments, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in various embodiments, have diameters of less than 50 microns, in various embodiments less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

5. Compositions for Other Routes of Administration

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in various embodiments, is about 2 to 3 gm.

Tablets and capsules for rectal administration are manufactured using the same pharmaceutically acceptable substance and by the same methods as for formulations for oral administration.

6. Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In various embodiments, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

7. Articles of Manufacture

The compounds or pharmaceutically acceptable derivatives may be packaged as articles of manufacture containing packaging material, a compound or pharmaceutically acceptable derivative thereof provided herein, which is effective for modulating α-synuclein fibril formation, or for treatment or amelioration of one or more symptoms of diseases or disorders in which α-synuclein fibril formation, is implicated, within the packaging material, and a label that indicates that the compound or composition, or pharmaceutically acceptable derivative thereof, is used for modulating α-synuclein fibril formation, or for treatment or amelioration of one or more symptoms of diseases or disorders in which α-synuclein fibril formation is implicated.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder in which α-synuclein fibril formation is implicated as a mediator or contributor to the symptoms or cause.

8. Sustained Release Formulations

Also provided are sustained release formulations to deliver the compounds to the desired target (i.e. brain or systemic organs) at high circulating levels (between 10⁻⁹ and 10⁻⁴ M). In a certain embodiment for the treatment of Alzheimer's or Parkinson's disease, the circulating levels of the compounds is maintained up to 10⁻⁷ M. The levels are either circulating in the patient systemically, or in various embodiments, present in brain tissue, and in a another embodiments, localized to the amyloid or α-synuclein fibril deposits in brain or other tissues.

It is understood that the compound levels are maintained over a certain period of time as is desired and can be easily determined by one skilled in the art. In various embodiments; the administration of a sustained release formulation is effected so that a constant level of therapeutic compound is maintained between 10⁻⁸ and 10⁻⁶ M between 48 to 96 hours in the sera.

Such sustained and/or timed release formulations may be made by sustained release means of delivery devices that are well known to those of ordinary skill in the art, such as those described in U.S. Pat. Nos. 3,845,770; 3,916,899; 3,536,809; 3, 598,123; 4,008,719; 4,710,384; 5,674,533; 5,059,595; 5,591,767; 5,120,548; 5,073,543; 5,639,476; 5,354,556 and 5,733,566, the disclosures of which are each incorporated herein by reference. These pharmaceutical compositions can be used to provide slow or sustained release of one or more of the active compounds using, for example, hydroxypropylmethyl cellulose, other polymer matrices, gels, permeable membranes, osmotic systems, multilayer coatings, microparticles, liposomes, microspheres, or the like. Suitable sustained release formulations known to those skilled in the art, including those described herein, may be readily selected for use with the pharmaceutical compositions provided herein. Thus, single unit dosage forms suitable for oral administration, such as, but not limited to, tablets, capsules, gelcaps, caplets, powders and the like, that are adapted for sustained release are contemplated herein.

In various embodiments, the sustained release formulation contains active compound such as, but not limited to, microcrystalline cellulose, maltodextrin, ethylcellulose, and magnesium stearate. As described above, all known methods for encapsulation which are compatible with properties of the disclosed compounds are contemplated herein. The sustained release formulation is encapsulated by coating particles or granules of the pharmaceutical compositions provided herein with varying thickness of slowly soluble polymers or by microencapsulation. In various embodiments, the sustained release formulation is encapsulated with a coating material of varying thickness (e.g. about 1 micron to 200 microns) that allow the dissolution of the pharmaceutical composition about 48 hours to about 72 hours after administration to a mammal. In another embodiment, the coating material is a food-approved additive.

In another embodiment, the sustained release formulation is a matrix dissolution device that is prepared by compressing the drug with a slowly soluble polymer carrier into a tablet. In various embodiments, the coated particles have a size range between about 0.1 to about 300 microns, as disclosed in U.S. Pat. Nos. 4,710,384 and 5,354,556, which are incorporated herein by reference in their entireties. Each of the particles is in the form of a micromatrix, with the active ingredient uniformly distributed throughout the polymer.

Sustained release formulations such as those described in U.S. Pat. No. 4,710,384, which is incorporated herein by reference in its entirety, having a relatively high percentage of plasticizer in the coating in order to permit sufficient flexibility to prevent substantial breakage during compression are disclosed. The specific amount of plasticizer varies depending on the nature of the coating and the particular plasticizer used. The amount may be readily determined empirically by testing the release characteristics of the tablets formed. If the medicament is released too quickly, then more plasticizer is used. Release characteristics are also a function of the thickness of the coating. When substantial amounts of plasticizer are used, the sustained release capacity of the coating diminishes. Thus, the thickness of the coating may be increased slightly to make up for an increase in the amount of plasticizer. Generally, the plasticizer in such an embodiment will be present in an amount of about 15 to 30% of the sustained release material in the coating, in various embodiments 20 to 25%, and the amount of coating will be from 10 to 25% of the weight of the active material, and in another embodiment, 15 to 20% of the weight of active material. Any conventional pharmaceutically acceptable plasticizer may be incorporated into the coating.

The compounds provided herein can be formulated as a sustained and/or timed release formulation. All sustained release pharmaceutical products have a common goal of improving drug therapy over that achieved by their non-sustained counterparts. Ideally, the use of an optimally designed sustained release preparation in medical treatment is characterized by a minimum of drug substance being employed to cure or control the condition. Advantages of sustained release formulations may include: 1) extended activity of the composition, 2) reduced dosage frequency, and 3) increased patient compliance. In addition, sustained release formulations can be used to affect the time of onset of action or other characteristics, such as blood levels of the composition, and thus can affect the occurrence of side effects.

The sustained release formulations provided herein are designed to initially release an amount of the therapeutic composition that promptly produces the desired therapeutic effect, and gradually and continually release of other amounts of compositions to maintain this level of therapeutic effect over an extended period of time. In order to maintain this constant level in the body, the therapeutic composition must be released from the dosage form at a rate that will replace the composition being metabolized and excreted from the body.

The sustained release of an active ingredient may be stimulated by various inducers, for example pH, temperature, enzymes, water, or other physiological conditions or compounds.

Preparations for oral administration may be suitably formulated to give controlled release of the active compound. In various embodiments, the compounds are formulated as controlled release powders of discrete microparticles that can be readily formulated in liquid form. The sustained release powder comprises particles containing an active ingredient and optionally, an excipient with at least one non-toxic polymer.

The powder can be dispersed or suspended in a liquid vehicle and will maintain its sustained release characteristics for a useful period of time. These dispersions or suspensions have both chemical stability and stability in terms of dissolution rate. The powder may contain an excipient comprising a polymer, which may be soluble, insoluble, permeable, impermeable, or biodegradable. The polymers may be polymers or copolymers. The polymer may be a natural or synthetic polymer. Natural polymers include polypeptides (e.g., zein), polysaccharides (e.g., cellulose), and alginic acid. Representative synthetic polymers include those described, but not limited to, those described in column 3, lines 33-45 of U.S. Pat. No. 5,354,556, which is incorporated by reference in its entirety. Particularly suitable polymers include those described, but not limited to those described in column 3, line 46-column 4, line 8 of U.S. Pat. No. 5,354,556 which is incorporated by reference in its entirety.

The sustained release compositions provided herein may be formulated for parenteral administration, e.g., by intramuscular injections or implants for subcutaneous tissues and various body cavities and transdermal devices. In various embodiments, intramuscular injections are formulated as aqueous or oil suspensions. In an aqueous suspension, the sustained release effect is due to, in part, a reduction in solubility of the active compound upon complexation or a decrease in dissolution rate. A similar approach is taken with oil suspensions and solutions, wherein the release rate of an active compound is determined by partitioning of the active compound out of the oil into the surrounding aqueous medium. Only active compounds which are oil soluble and have the desired partition characteristics are suitable. Oils that may be used for intramuscular injection include, but are not limited to, sesame, olive, arachis, maize, almond, soybean, cottonseed and castor oil.

A highly developed form of drug delivery that imparts sustained release over periods of time ranging from days to years is to implant a drug-bearing polymeric device subcutaneously or in various body cavities. The polymer material used in an implant, which must be biocompatible and nontoxic, include but are not limited to hydrogels, silicones, polyethylenes, ethylene-vinyl acetate copolymers, or biodegradable polymers.

### E. Evaluation of the Activity of the Compounds

The activity of the compounds provided herein as modulators of α-synuclein toxicity may be measured in standard assays (see, e.g., U.S. patent application Ser. No. 10/826,157, filed Apr. 16, 2004; U.S. Patent Application Publication No. 2003/0073610; and EXAMPLE 1 herein). The activity may be measured in a whole yeast cell assay using 384-well screening protocol and an optical density measurement. Expression of human α-synuclein in yeast inhibits growth in a copy-number dependent manner (see, e.g., Outeiro, et al. (2003) Science 302(5651):1772-5). Expression of one copy of α-syn::GFP has no effect on growth, while two copies result in complete inhibition. The cessation of growth is accompanied by a change in α-syn::GFP localization. In cells with one copy, α-syn::GFP associates with the plasma membrane in a highly selective manner. When expression is doubled, α-synuclein migrates to the cytoplasm where it forms large inclusions that are similar to Lewy bodies seen in diseased neurons.

The compounds provided herein were screened in this assay for α-synuclein toxicity rescue. Briefly, the humanized strain is exposed to compounds in 384-well plates under conditions that induce α-synuclein expression. After incubation for 24 or 48 hours, or both, growth is measured. Compounds that inhibit toxicity will restore growth and are detected as an increase in turbidity (OD₆₀₀).

Additional assays can be used to screen compounds to assess their ability to modulate α-synuclein toxicity. These assays include, for example, screening for compounds that modulate α-synuclein induced toxicity in human neuroglioma cells (see, e.g., McLean et al. (2004) Biochem Biophys Res Commun. 321(3):665-69) or in worms or primary neurons (see, e.g., Cooper et al. (2006) Science 313(5785):324-8 and supplementary materials).

### F. Methods of Use of the Compounds and Compositions

Provided herein are methods to inhibit or prevent α-synuclein toxicity and/or fibril formation, methods to inhibit or prevent α-synuclein fibril growth, and methods to cause disassembly, disruption, and/or disaggregation of α-synuclein fibrils and α-synuclein-associated protein deposits. The methods can be in vitro or in vivo methods.

In certain embodiments, the synuclein diseases or synucleinopathies treated or whose symptoms are ameliorated by the compounds and compositions provided herein include, but are not limited to diseases associated with the formation, deposition, accumulation, or persistence of synuclein fibrils, including α-synuclein fibrils. In certain embodiments, such diseases include Parkinson's disease, familial Parkinson's disease, Lewy body disease, the Lewy body variant of Alzheimer's disease, dementia with Lewy bodies, multiple system atrophy, and the Parkinsonism-dementia complex of Guam.

In practicing the in vitro methods, varying amounts of the compounds or compositions provided herein can be contacted with a cell, e.g., a cell, such as a yeast cell expressing human α-synuclein, and the effects of the compound evaluated. In practicing the in vivo methods, effective amounts of the compounds or compositions provided herein are administered to a mammal, e.g., a human, cow, horse, pig, monkey, rat, mouse, sheep, dog, cat, or rabbit. Such amounts are sufficient to achieve a therapeutically effective concentration of the compound or active component of the composition in vivo.

### G. Combination Therapy

The compounds and compositions provided herein may also be used in combination with other active ingredients. In another embodiment, the compounds may be administered in combination, or sequentially, with another therapeutic agent. Such other therapeutic agents include those known for treatment or amelioration of one or more symptoms of α-synuclein diseases. Such therapeutic agents include, but are not limited to, donepezil hydrochloride (Aracept), rivastigmine tartrate (Exelon), tacrine hydrochloride (Cognex) and galantamine hydrobromide (Reminyl).

### EXAMPLES

The following examples are provided for illustrative purposes only and are not intended to limit the scope of the invention.

### Example 1

### α-Synuclein (aS) Screening

Yeast Strains

Parental W303: MAT a/α ade2-1/ade2-1 his3-11,15/his3-11,15 leu2-3,112/leu2-3,112

trp1-1/trp1-1 ura3-1/ura3-1 can1-100/can1-100

Phenotype: Requires adenine, histidine, leucine, tryptophan, and uracil for growth. Resistant to canavanine.

Fx-109: MAT a/α ade2-1/ade2-1 his3-11,15/his3-11,15 leu2-3,112/leu2-3,112

trp 1-1/trp1-1 GALp-aS-GFP::TRP1/GALp-aS-GFP::TRP1 ura3-1/ura3-1

GALp-aS-GFP::URA3/GALp-aS-GFP::URA3 can1-100/can1-100 pdr1::KanMX/pdr1::KanMX erg6::KanMX/erg6::KanMX

Phenotype: Unable to grow on galactose due to expression of aS. Requires histidine, leucine, and adenine for growth. Resistant to canavanine and kanamycin. Hypersensitive to drugs.

Media and Reagents

Based on the genotype of the strain to be tested, choose the appropriate supplementation for the synthetic media. Strains containing integrated constructs (eg, aS) should be grown in medium which maintains selection for the construct (see below). CSM (Qbiogene) is a commercially-available amino acid mix for growing Saccharomyces cerevisiae. It can be obtained lacking one or more amino acids as required. For the aS and control strains, media lacking tryptophan and uracil (-Trp-Ura) should be used (available from Qbiogene, Inc., Carlsbad, Calif.).

To make liquid synthetic medium, mix the components listed in Tables V, VI, and VII. After the components have dissolved, sterilize by filtration (Millipore Stericup Cat#SCGPU11RE) into a sterile bottle.

TABLE V
Synthetic Complete Medium

| | | Catalogue | | | | | |
|---|---|---|---|---|---|---|---|
| Component | Vendor | # | Size | Amount per L | | Final Conc. | |
| Yeast Nitrogen Base | Difco | 291920 | 2 kg | 6.7 | g | 0.67% | (w/v) |
| without amino acids | | | | | | | |
| Carbon source: one of | See | See | See | 20 | g | 2% | (w/v) |
| glucose, galactose, | below | below | below | | | | |
| raffinose-see Table VI | | | | | | | |

| | | | | |
|---|---|---|---|---|
| CSM: strain | Qbiogene | See | See | ~0.8 g |
| determines type-see | | below | below | (according to |
| Table VII | | | | manufacturer) |

| | | | | | |
|---|---|---|---|---|---|
| MilliQ Water | — | — | 1 | L | — |

TABLE VI
Carbon Sources

| | | | | | | |
|---|---|---|---|---|---|---|
| Glucose (also known | Fisher | D16-10 | 10 | kg | 20 g | 2% (w/v) |
| as dextrose) | | | | | | |
| Galactose | SIGMA | G-0750 | 1 | kg | 20 g | 2% (w/v) |
| Raffinose | Difco | 217410 | 100 | g | 20 g | 2% (w/v) |

TABLE VII
CSM

| | | | | | |
|---|---|---|---|---|---|
| CSM-Trp-Ura | Qbiogene | 4520-522 | 100 g | 0.72 g | See Qbiogene |
| for aS and | | | | | web page |
| control strain | | | | | |
| CSM for the | Qbiogene | 4500-022 | 100 g | 0.79 g | See Qbiogene |
| parental | | | | | web page |
| strain | | | | | |

384-Well Screening Protocol Using Optical Density

Day 1

Innoculate an appropriate volume of SRaffinose-Trp-Ura medium with Fx-109 strain.

Incubate with shaking at 30° C. overnight until cells reach log or mid-log phase (OD₆₀₀ 0.5-1.0; 0.1 OD600 corresponds to ˜1.75×10 E6 cells).

Day 2

Spin down cells at room temperature, remove medium, and resuspend in an equivalent volume of SGalactose-Trp-Ura medium. Measure the OD₆₀₀ and dilute cells to 0.001. Robotically transfer 30 μl of cell suspension (MicroFill, Biotek) to each well of a 384-well plate (NUNC 242757).

Add 100 n1 drug in DMSO (Cybio) to each well (final conc. 17 μg/ml drug and 0.333% DMSO)

For the positive controls add glucose to final concentrations of 0.1% and 1%. (Note: daunorubicin may be an additional control based on Biochem J. 368:131-6, 2002, but we have not tested it.)

Incubate plates at 30° C. without shaking in a humidified chamber for 24 and/or 48 hours.

Day 3 (24 Hours Later) and/or Day 4 (48 Hours Later)

Read OD₆₅₀ (Envision, Perkin Elmer) and also visually inspect wells for growth of yeast culture.

Results

The compounds provided herein were assayed as described above and showed an MRC (minimum rescue concentration) of less than about 300 μM.

### Example 2

Compounds according to Formula I were prepared using the schemes and processes described above and/or set forth below.

Process A, also described above under Preparation of the Compounds, was used to prepare the following compound:

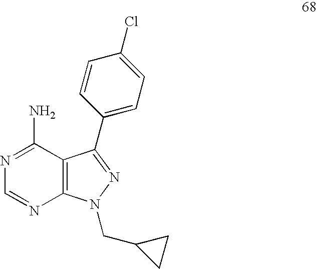

Step A:

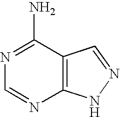

A mixture of commercially available 5-amino-1H-pyrazole-4-carbonitrile (16.22 g, 0.15 mol) and formamide (84.6 ml) was heated at 180° C. for 4 hr under a nitrogen atmosphere. The solution was cooled to ambient temperature and the crystals were separated, washed with water and dried to afford the product (18.6 g, 91%).

Step B:

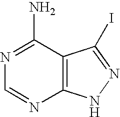

A mixture of 1H-Pyrazolo[3,4-d]pyrimidin-4-ylamine (11.75 g, 0.09 mol) (Step A) and N-iodosuccinimide (25.45 g, 0.11 mol) in dimethylformamide (300 ml) was stirred at 50° C. for 24 hr. A second batch of N-iodosuccinimide (3.92 g, 0.02 mol) was added and the solution stirred for additional 24 hr. Upon standing at room temperature, a precipitate was formed which was separated by filtration and washed with dimethylformamide and ethanol to afford 10.05 g of the title compound. The filtrate was concentrated in vacuo to about one half of the original volume and 500 ml of water was added. The precipitated product was separated by filtration and washed with ethanol to afford a second batch of the product (10.53 g, combined yield 20.58 g, 90.6%); LC/MS, API-ES, Pos, (M+H)⁺, 262.1.

Step C:

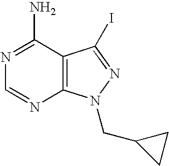

3-Iodo-1H-pyrazolo[3,4-d]pyrimidin-4-ylamine (1.0 g, 3.83 mmol) (Step B), cyclopropyl-methanol (0.83 g, 11.51 mmol) and triphenylphosphine (2.01 g, 7.66 mmol) were dissolved in anhydrous tetrahydrofuran (50 ml) and stirred at 0° C. Diethylazodicarboxylate (1.33 g, 7.63 mmol) was slowly added and the solution stirred at 0° C. for 15 min. Solution was allowed to warm to room temperature and stirred for 1 hr. Solvent was evaporated in vacuo and product adsorbed on silica gel. Flash chromatography on silica gel (eluent, hexane:ethyl acetate, 50:50 to 20:80) followed by trituration with acetonitrile afforded the title compound (0.77 g, 63.6%); LC/MS, API-ES, Pos, (M+H)⁺, 316.1.

Step D:

1-Cyclopropylmethyl-3-iodo-1H-pyrazolo[3,4-d]pyrimidin-4-ylamine (0.12 g, 0.38 mmol) (Step C), 4-chlorophenylboronic acid (0.65 g, 0.42 mmol), tetrakistriphenylphosphine palladium (0.03 g, 0.02 mmol) and sodium carbonate (0.09 g, 0.85 mmol) were mixed in 1,2-dimethoxyethane (10 ml) and water (5 ml) and the solution refluxed under argon for 6 hr. Water was added and the product was extracted with ethyl acetate (2×25 ml). Evaporation of the solvent followed by flash chromatography on silica gel (eluent, hexane:ethyl acetate, 50:50 to 10:90) afforded the title compound (0.04 g, 35.1%); LC/MS, API-ES, Pos, (M+H)⁺, 300.1.

Process B, also described above under Preparation of the Compounds, was used to prepare the following compound:

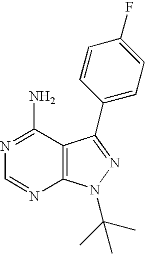

Step A:

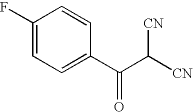

To a stirred solution of malononitrile (2.08 g, 31.5 mmol) in 50 ml of anhydrous tetrahydrofuran at 0° C. was slowly added sodium hydride (60%, 2.52 g, 63 mmol) in portions and solution stirred for 10 min. A solution of 4-fluorobenzoyl chloride (5.0 g, 31.5 mmol) in tetrahydrofuran (25 ml) was slowly added via an addition funnel and solution stirred at ambient temperature for 1 hr. Dilute hydrochloric acid (1 mol/L, 100 ml) was added and the product extracted with ethyl acetate. The organic layer was washed with water, brine, and evaporated to afford a residue which was triturated with hexane to afford the title compound (4.98 g, 83.9%); LC/MS, API-ES, Neg, (M−H)⁻, 187.0.

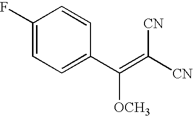

Step B:

2-(4-Fluoro-benzoyl)-malononitrile (4.98 g, 26.47 mmol) (Step A) was dissolved in a mixture of anhydrous acetonitrile (100 ml) and methanol (10 ml) and trimethylsilyl diazomethane (2M solution in diethyl ether, 19.9 ml, 39.8 mmol) was added. Solution was stirred at 0° C. under a nitrogen atmosphere and N,N-diisopropylethylamine (6.84 g, 52.9 mmol) was slowly added. The solution was stirred at ambient temperature for 18 hr and solvent evaporated in vacuo. The residue was adsorbed on silica gel and purified by chromatography (eluent, hexane:ethyl acetate, 80:20 to 70:30) to afford the title compound (2.83 g, 52.9%) as and oil; LC/MS, API-ES, Pos, (M+H)⁺, 203.0).

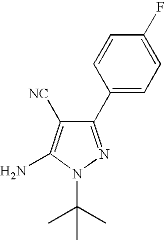

Step C:

2-[(4-Fluoro-phenyl)-methoxy-methylene]-malononitrile (2.80 g, 13.85 mmol) (Step B) was dissolved in anhydrous ethanol (75 ml) and t-butylhydrazine hydrochloride (1.73 g, 13.88 mmol) was added. The solution was refluxed for 2 hr and solvent evaporated. The product was purified by flash column chromatography on silica gel (eluent, hexane:ethyl acetate, 80:20 to 30:70) to afford the title compound (3.02 g, 84.4%); LC/MS, API-ES, Pos, (M+H)⁺, 259.1).

Step D: 5-Amino-1-tert-butyl-3-(4-fluoro-phenyl)-1H-pyrazole-4-carbonitrile (0.82 g, 3.16 mmol) was mixed with formamide (5 ml) and the mixture heated at 180° C. under a nitrogen atmosphere for 3 hr. Upon cooling, the product separated as crystalline material which was separated by filtration, washed with water and dried to afford the title compound (0.73 g, 81.1%); LC/MS, API-ES, Pos, (M+H)⁺, 286.1.

Process C, also described above under Preparation of the Compounds, was used to prepare the following compound:

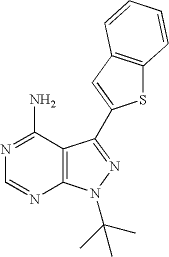

Step A:

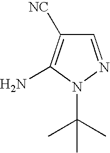

A mixture of t-butylhydrazine hydrochloride (4.67 g, 53 mmol) and triethylamine (5.35 g, 53 mmol) in anhydrous ethanol (250 ml) was stirred and ethoxymethylene malononitrile (6.47 g, 53 mmol) was slowly added in portions. The mixture was heated at reflux for 3 hr. The solvent was removed in vacuo and the product was crystallized from ethyl acetate-hexane followed by ether to afford the title compound as light pale brown crystals (5.6 g, 64.4%); LC/MS, API-ES, Neg, (M−H)−, 163.0.

Step B:

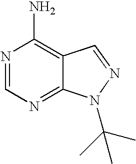

A mixture of 5-amino-1-tert-butyl-1H-pyrazole-4-carbonitrile (5.5 g, 33.5 mmol) (Step A) and formamide (68 ml) was heated at 185° C. for 3 hr under nitrogen atmosphere. The mixture was added to water and extracted with ethyl acetate. The organic layer was washed with saturated sodium bicarbonate solution followed by aqueous wash and brine. The organic layer was dried (anhydrous sodium sulfate) and the solvent was removed in vacuo to afford a residue which was crystallized from small amount of ether to afford the title compound (3.91 g, 60.9%); LC/MS, API-ES, Pos, (M+H)⁺, 192.1.

Step C:

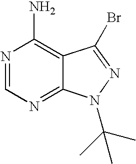

1-tert-Butyl-1H-pyrazolo[3,4-d]pyrimidin-4-ylamine (1.6 g, 8.37 mmol) (Step B) was suspended in water (30 ml) and bromine (2.68 g, 16.7 mmol) was added. The mixture was stirred at ambient temperature for 1 hr followed by stirring at 100° C. for 1 hr. After cooling, the precipitated product was separated by filtration. The residue was stirred in 50 ml of 5% aqueous sodium hydrogen sulfite solution for 0.5 hr and the solution was treated with 10 ml of saturated aqueous sodium bicarbonate. The precipitate was separated by filtration, washed with water and dried to afford the title compound (1.46 g, 64.6%); LC/MS, API-ES, Pos, (M+H)⁺, 270.0 and 272.0.

Step D: 3-Bromo-1-tert-butyl-1H-pyrazolo[3,4-d]pyrimidin-4-ylamine (351 mg, 1.3 mmol) (Step C), thianaphthene-2-boronic acid (255 mg, 1.43 mmol), tetrakistriphenylphosphine palladium (90 mg, 0.07 mmol) and sodium carbonate (330 mg, 3.11 mmol) were mixed in 1,2-dimethoxyethane (20 ml) and water (10 ml) and the solution refluxed under argon for 6 hr. Water was added and the product was extracted with ethyl acetate (2×25 ml). Evaporation of the solvent followed by flash chromatography on silica gel (eluent, hexane:ethyl acetate, 80:20 to 65:35) afforded the title compound as an off-white powder (136 mg, 31.5%); LC/MS, API-ES, Pos, (M+H)⁺, 324.1.

Process D, also described above under Preparation of the Compounds, was used to prepare the following compound:

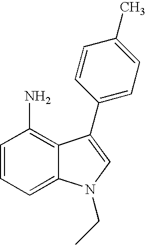

Step A:

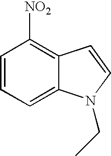

To a stirred solution of 4-nitroindole (2.5 g, 15.4 mmol) in 50 ml acetone at 0° C. was added 4.32 g (76.9 mmol) powdered potassium hydroxide and the solution stirred for 5 min. Ethyl iodide (4.8 g, 30.8 mmol) was added and the solution stirred vigorously for 15 min at ambient temperature. Toluene (300 ml) was added and the insoluble material was removed by filtration. The solution was washed with 5% aqueous citric acid followed by water, dried (anhydrous sodium sulfate) and solvent removed in vacuo. Residue was triturated with hexane-ethyl acetate (7:3) to afford the title compound (2.6 g, 88.7%); LC/MS, API-ES, Pos, (M+H)⁺, 191.1.

Step B:

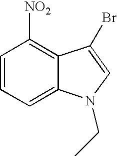

A solution of 1-ethyl-4-nitro-1H-indole (2.93 g, 15.4 mmol) (Step A) in anhydrous tetrahydrofuran (100 ml) was stirred at −78° C. N-bromosuccinimide (3.56 g, 20.0 mmol) was slowly added and the solution stirred at this temperature for 2 hr. Silica gel (8.0 g) was added and the solution evaporated in vacuo to afford a slurry that was flash chromatographed on silica gel (eluent, hexane:ethyl acetate, 90:10 to 80:20). The title compound was isolated as a pale yellow solid (2.48 g, 59.9%); LC/MS, API-ES, Pos, (M+H)⁺, 269.0 and 271.0.

Step C:

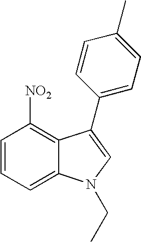

3-Bromo-1-ethyl-4-nitro-1H-indole (349.8 mg, 1.3 mmol) (Step B), 4-methylphenylboronic acid (194.4 mg, 1.43 mmol), tetrakistriphenylphosphine palladium (90.1 mg, 0.08 mmol) and sodium carbonate (330.7 mg, 3.12 mmol) were mixed in 1,2-dimethoxyethane (20 ml) and water (10 ml) and the solution refluxed under argon for 6 hr. Water was added and the product was extracted with ethyl acetate (3×25 ml). Evaporation of the solvent followed by flash chromatography on silica gel (eluent, hexane:ethyl acetate, 90:10 to 80:20) afforded the title compound (220 mg, 60.4%); LC/MS, API-ES, Pos, (M+H)⁺, 281.1.

Step D: 1-Ethyl-4-nitro-3-p-tolyl-1H-indole (220 mg, 0.78 mmol) (Step C) was dissolved in a mixture of methanol and ethyl acetate (3:1, 50 ml) and 10% Pd/C (22 mg) was added. Hydrogen gas was bubbled gently through the solution for 2 hr. The catalyst was removed by filtration and the solvent evaporated. The product was purified by flash chromatography on silica gel (eluent, hexane:ethyl acetate, 90:10 to 80:20) to afforded the title compound (65 mg, 33.3%) as a colorless oil; LC/MS, API-ES, Pos, (M+H)⁺, 251.2.

Process E, also described above under Preparation of the Compounds, was used to prepare the following compound:

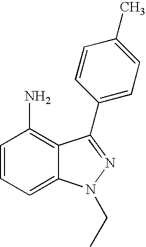

Step A:

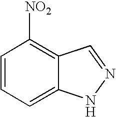

A solution of 2-methyl-3-nitro-phenylamine (5.5 g, 36.15 mmol) in glacial acetic acid (250 ml) was stirred at 0° C. Sodium nitrite (2.5 g, 36.15 mmol) dissolved in water (6 ml) was added to the stirred solution all at once and the stirring continued for 15 min. Yellow precipitate was removed by filtration and discarded and the solution stirred at ambient temperature for 4 hr. Solvent was removed in vacuo and water (20 ml) was added. The precipitate was separated by filtration and dried to afford the crude product. Chromatographic purification on silica gel (eluent, hexane:ethyl acetate, 70:30 to 50:50) afforded the title compound (4.0 g, 67.8%).

Step B:

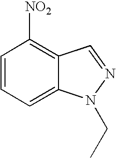

Sodium hydride (60%, 0.40 g, 10 mmol) was suspended in anhydrous dimethylformamide (8 ml) and stirred at −10° C. 4-Nitro-1H-indazole (1.0 g, 6.13 mmol) (Step A) dissolved in dimethylformamide (8 ml) was slowly added and the solution stirred for 20 min at this temperature. Ethyl iodide (1.05 g, 6.73 mmol) was added drop-wise and the solution stirred at ambient temperature for 2 hr. The solution was then poured on to ice-water and product extracted with methylene chloride. TLC and LC-MS analysis indicated the presence of two isomeric products that were separated by column chromatography on silica gel (eluent, hexane:ethyl acetate, 80:20 to 60:40) to afford the title compound 1-ethyl-4-nitro-1H-indazole (0.43 g, 37.0%), LC/MS, API-ES, Pos, (M+H)⁺, 192.1, and the isomeric 2-ethyl-4-nitro-2H-indazole (0.48 g, 41.1%); LC/MS, API-ES, Pos, (M+H)⁺, 192.1.

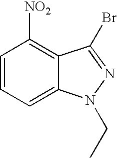

Step C:

1-Ethyl-4-nitro-1H-indazole (0.43 g, 2.26 mmol) (Step B) was dissolved in glacial acetic acid (15 ml) and bromine (0.47 g, 2.94 mmol) was added. The solution was stirred at 80° C. for 30 min and a second batch of bromine (0.11 g, 0.68 mmol) was added and the solution stirred for an additional 30 min. Solution was added to a saturated aqueous solution of sodium bicarbonate and the product extracted with dichloromethane. Organic layer was washed with water and dried (anhydrous magnesium sulfate) and solvent evaporated in vacuo to afford a crude product. The title compound was purified, by flash column chromatography on silica gel (eluent, hexane:ethyl acetate, 80:20 to 70:30) (0.59 g, 96.7%); LC/MS, API-ES, Pos, (M+H)⁺, 270.0 and 272.0.

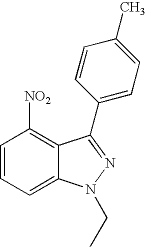

Step D:

3-Bromo-1-ethyl-4-nitro-1H-indazole (0.59 g, 2.18 mmol) (Step C), 4-methylphenylboronic acid (0.36 g, 2.65 mmol), tetrakistriphenylphosphine palladium (0.15 g, 0.13 mmol) and sodium carbonate (0.55 g, 5.19 mmol) were mixed in 1,2-dimethoxyethane (20 ml) and water (10 ml) and the solution refluxed under argon for 8 hr. Water was added and the product was extracted with ethyl acetate (3×25 ml). Evaporation of the solvent followed by flash chromatography on silica gel (eluent, hexane:ethyl acetate, 90:10 to 80:20) afforded the title compound (0.50 g, 81.5%); LC/MS, API-ES, Pos, (M+H)⁺, 282.1.

Step E: 1-Ethyl-4-nitro-3-p-tolyl-1H-indazole (0.50 g, 1.77 mmol) (Step D) was dissolved in a mixture of methanol (80 ml) and ethyl acetate (20 ml) and 10% Pd/C (50 mg) was added. Hydrogen gas was gently bubbled through the solution with stirring at ambient temperature for 2 hr. The catalyst was removed by filtration over celite and the filtrate was evaporated in vacuo. Purification by flash chromatography on silica gel (eluent, hexane:ethyl acetate, 90:10 to 85:15) afforded the title compound (0.33 g, 74.1%); LC/MS, API-ES, Pos, (M+H)⁺, 252.1.

Process F, described below, was used to prepare the following compound:

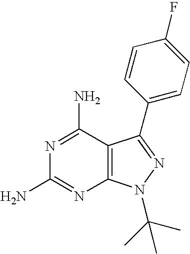

A mixture of 5-amino-1-tert-butyl-3-(4-fluoro-phenyl)-1H-pyrazole-4-carbonitrile (1.0 g, 3.87 mmol), guanidine carbonate (1.22 g, 6.77 mmol) and triethylamine (5 ml) was heated in a sealed tube at 205° C. for 2.5 hr. Water was added and the product extracted with ethyl acetate (4×30 ml). The organic layer was washed with water and brine, dried (anhydrous sodium sulfate) and evaporated. A fraction of the crude product (¼) was subjected to reverse phase HPLC and the desired peak was pooled (water-acetonitrile gradient, 0.05% trifluoroacetic acid, 70:30 to 10:90, 20 min, linear gradient; flow, 15 ml/min; column, Phenomenex Luna 5μ C18, 100×21.2 mm; UV 254 and 218 nm). Evaporation of the solvent followed by crystallization from ether afforded the title compound (55 mg, 18.9%); LC/MS, API-ES, Pos, (M+H)⁺, 301.1.

Process G, described below, was used to prepare the following compounds:

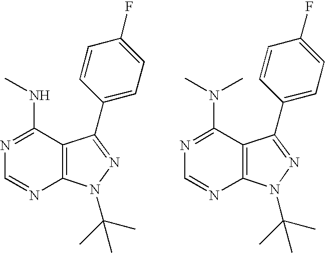

Sodium hydride (60%, 22 mg, 0.55 mmol) was suspended in anhydrous dimethylformamide (5 ml) and stirred at 0° C. 1-Tert-butyl-3-(4-fluoro-phenyl)-1H-pyrazolo[3,4-d]pyrimidin-4-ylamine (142.6 mg, 0.5 mmol) dissolved in 1 ml dimethylformamide was added and the solution stirred for 10 min. Methyl iodide (354.9 mg, 2.5 mmol) was added and the solution stirred at ambient temperature over night. Water was added and the product extracted with ethyl acetate. Organic layer was washed with water and brine, dried (anhydrous sodium sulfate) and evaporated to afford a product mixture. Flash chromatography on silica gel (eluent, hexane:ethyl acetate, 90:10 to 70:30) afforded the title compounds [1-tert-butyl-3-(4-fluoro-phenyl)-1H-pyrazolo[3,4-d]pyrimidin-4-yl]-dimethyl-amine (66.5 mg, 42.4%), LC/MS, API-ES, Pos, (M+H)⁺, 314.1 and [1-tert-butyl-3-(4-fluoro-phenyl)-1H-pyrazolo[3,4-d]pyrimidin-4-yl]-methyl-amine (41.5 mg, 27.7%), LC/MS, API-ES, Pos, (M+H)⁺, 300.1.

Process H, described below, was used to prepare the following compounds:

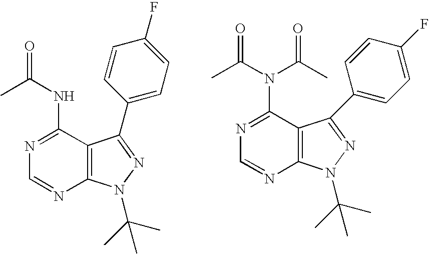

1-Tert-butyl-3-(4-fluoro-phenyl)-1H-pyrazolo[3,4-d]pyrimidin-4-ylamine (142.6 mg, 0.5 mmol) was dissolved in 2 ml of anhydrous pyridine and solution stirred at 0° C. Acetyl chloride (196.3 mg, 2.5 mmol) was added drop-wise and the solution stirred at ambient temperature over night. Water was added and the product extracted with ethyl acetate. Organic layer was washed with water and brine, dried (anhydrous sodium sulfate) and evaporated to afford a product mixture. Flash chromatography on silica gel (eluent, hexane:ethyl acetate, 90:10 to 70:30) afforded the title compounds N-acetyl-N-[1-tert-butyl-3-(4-fluoro-phenyl)-1H-pyrazolo[3,4-d]pyrimidin-4-yl]-acetamide (45.0 mg, 24.3%), LC/MS, API-ES, Pos, (M+H)⁺370.1, and N-[1-tert-butyl-3-(4-fluoro-phenyl)-1H-pyrazolo[3,4-d]pyrimidin-4-yl]-acetamide (12.7 mg, 7.8%), LC/MS, API-ES, Pos, (M+H)⁺328.1.

Process I, described below, was used to prepare the following compound:

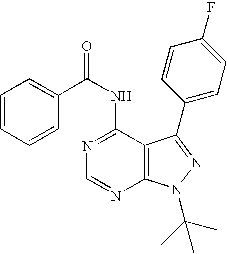

1-Tert-butyl-3-(4-fluoro-phenyl)-1H-pyrazolo[3,4-d]pyrimidin-4-ylamine (142.6 mg, 0.5 mmol) was dissolved in 2 ml of anhydrous pyridine and solution stirred at 0° C. Benzoyl chloride (351.4 mg, 2.5 mmol) was added drop-wise and the solution stirred at ambient temperature over night. Water was added and the product extracted with ethyl acetate. Organic layer was washed with water and brine, dried (anhydrous sodium sulfate) and evaporated to afford a product mixture. The residue was stirred in acetonitrile and the precipitate was separated by filtration. Flash chromatography on silica gel (eluent, hexane:ethyl acetate, 90:10 to 70:30) afforded the title compound (75.0 mg, 38.5%), LC/MS, API-ES, Pos, (M+H)⁺390.1.

Process J, described below, was used to prepare the following compound:

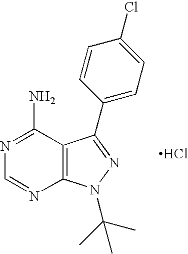

1-Tert-butyl-3-(4-chloro-phenyl)-1H-pyrazolo[3,4-d]pyrimidin-4-ylamine (100 mg, 0.33 mmol) was dissolved in 3 ml of anhydrous chloroform and ethereal HCl (1M solution, 0.4 ml, 0.4 mmol) was added. The solution was allowed to stand at ambient temperature for 1 hr. Upon partial evaporation of the solvent, a precipitate was formed that was separated by decantation and the residue washed with small amount of ether and dioxane to afford the title compound (80 mg, 71.6%), LC/MS, API-ES, Pos, (M+H)⁺, parent ion for free base, 302.1.

Process K, described below, was used to prepare the following compound:

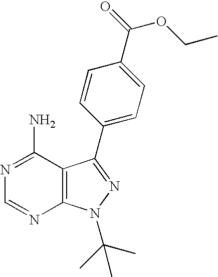

3-Bromo-1-tert-butyl-1H-pyrazolo[3,4-d]pyrimidin-4-ylamine (351 mg, 1.3 mmol), ethyl 4-(4,4,5,5-tetramethyl-1,3,2-dioxaborolan-2-yl)benzoate (395 mg, 1.43 mmol), tetrakistriphenylphosphine palladium (90 mg, 0.07 mmol) and sodium carbonate (330 mg, 3.11 mmol) were mixed in 1,2-dimethoxyethane (20 ml) and water (10 ml) and the solution refluxed under argon for 6 hr. Water was added and the product was extracted with ethyl acetate (3×25 ml). Evaporation of the solvent followed by flash chromatography on silica gel (eluent, hexane:ethyl acetate, 80:20 to 60:40) afforded the title compound that was crystallized form methanol (80 mg, 18.1%); LC/MS, API-ES, Pos, (M+H)⁺, 340.1.

Process L, also described above under Preparation of the Compounds, was used to prepare the following compound:

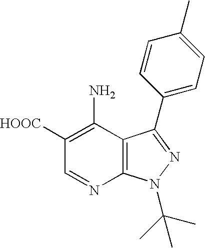

Step A:

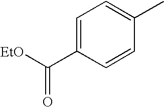

Thionyl chloride (22.3 ml, 0.3 mol) was added to 4-methyl-benzoic acid (27.1 g, 0.2 mol) in ethanol (200 ml) and the solution stirred overnight. The solvent was evaporated to give 4-methyl-benzoic acid ethyl ester (30 g, 91%) as a viscous liquid.

Step B:

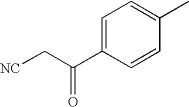

To a stirred solution of acetonitrile (48 ml, 0.92 mol) and toluene (100 ml), sodium hydride (22 g, 0.92 mol) was added in parts. After stirring at 50° C. for 2 hr, 4-methyl-benzoic acid ethyl ester (30 g, 0.18 mol) (Step A) in toluene (100 ml) was added and refluxed for 4 hr. The solvents were then evaporated under vacuum. The residue was quenched with ice (200 ml) and extracted with ethyl acetate. The organic layer was washed with brine, dried over anhydrous sodium sulfate, concentrated, and purified by column chromatography to give 3-oxo-3-p-tolyl-propionitrile, 22 g (77%).

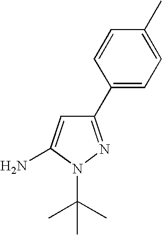

Step C:

3-Oxo-3-p-tolyl-propionitrile (22 g, 0.14 mol) (Step B) was dissolved in isopropanol (500 ml), triethylamine (40 ml, 0.28 mol) was added, and the mixture was stirred for 5 min, then t-butyl hydrazine hydrochloride was added, and the mixture was refluxed for 5 hr under nitrogen. The reaction was cooled to room temperature and the solvent was removed in vacuo. The residue was dissolved in ethyl acetate, washed with water, brine, and dried over anhydrous sodium sulfate. The organic layer was filtered, concentrated under vacuum, loaded on a silica gel column and purified to give 2-tert-butyl-5-p-tolyl-2H-pyrazol-3-ylamine, 24 g (75%).

Step D:

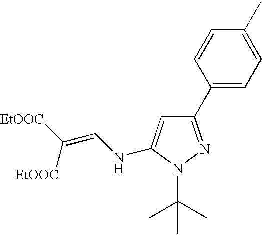

2-Tert-butyl-5-p-tolyl-2H-pyrazol-3-ylamine (10 g, 0.044 mol) (Step C) was stirred with diethyl(ethoxymethylene)malonate (9.5 g, 0.044 mol) at 120° C. for 4 hr. The mixture was dissolved in dichloromethane, adsorbed on silica gel and purified by column chromatography to give 2-[(2-tert-butyl-5-p-tolyl-2H-pyrazol-3-ylamino)-methylene]-malonic acid diethyl ester, 10 g (57%).

Step E:

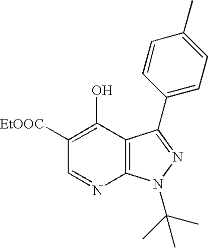

2-[(2-Tert-butyl-5-p-tolyl-2H-pyrazol-3-ylamino)-methylene]-malonic acid diethyl ester (5 g, 12.5 mmol) (Step D) was stirred in diphenyl ether (75 ml) at 190° C. for 48 hr. The resultant solution was cooled to room temperature, poured slowly on to a silica gel column and eluted with petroleum ether to give 1-tert-butyl-4-hydroxy-3-p-tolyl-1H-pyrazolo[3,4-b]pyridine-5-carboxylic acid ethyl ester, 1.1 g (25%).

Step F:

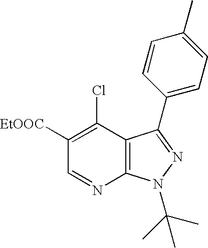

1-Tert-butyl-4-hydroxy-3-p-tolyl-1H-pyrazolo[3,4-b]pyridine-5-carboxylic acid ethyl ester (1.1 g, 3.1 mmol) (Step E) was refluxed in POCl₃ for 4 hr. The mixture was concentrated under vacuum to remove POCl₃. The residue was diluted with water and extracted with ethyl acetate. The extracts were dried (anhydrous sodium sulfate), filtered and the filtrate was concentrated and purified by column chromatography to give 1-tert-butyl-4-chloro-3-p-tolyl-1H-pyrazolo[3,4-b]pyridine-5-carboxylic acid ethyl ester, 0.8 g (69%).

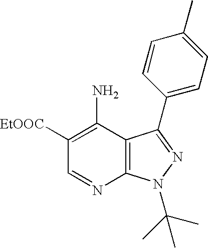

Step G:

1-Teri-butyl-4-chloro-3-p-tolyl-1H-pyrazolo[3,4-b]pyridine-5-carboxylic acid ethyl ester (0.8 g, 2.2 mmol) was stirred in 25 ml of ethanol saturated with ammonia in a closed steel vessel at 110° C. for 12 hr. The cooled reaction mixture was concentrated and the residue was triturated with ether and filtered. The filtrate was dried (anhydrous sodium sulfate), filtered, concentrated, and purified by column chromatography to give 4-amino-1-tert-butyl-3-p-tolyl-1H-pyrazolo[3,4-b]pyridine-5-carboxylic acid ethyl ester, 0.5 g (66%).

Step H: 4-Amino-1-tert-butyl-3-p-tolyl-1H-pyrazolo[3,4-b]pyridine-5-carboxylic acid ethyl ester (0.5 g, 1.4 mmol) was stirred in ethanol (95%) and sodium hydroxide (0.24 g, 6.0 mmol) overnight at 50° C. The mixture was concentrated, the residue dissolved in water (600 ml), filtered and acidified with acetic acid. The precipitate formed was collected, washed with water and air dried to give 4-amino-1-tert-butyl-3-p-tolyl-1H-pyrazolo[3,4-b]pyridine-5-carboxylic acid, 0.3 g (66%) as a white solid; LC/MS, APCI, Neg, (M−H)−, 323.3.

Process M, described below, was used to prepare the following compound:

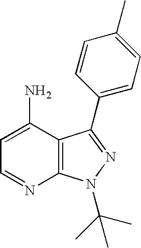

4-Amino-1-tert-butyl-3-p-tolyl-1H-pyrazolo[3,4-b]pyridine-5-carboxylic acid (Example 12) (0.1 g, 0.3 mmol) was heated at 180° C. under a nitrogen atmosphere for 48 hr. The resulting product was purified by column chromatography to give 20 mg (21%) of 1-tert-butyl-3-p-tolyl-1H-pyrazolo[3,4-b]pyridine-4-ylamine as pale brown solid; LC/MS, APCI, Pos, (M+H)⁺, 281.5.

Since modifications will be apparent to those of skill in the art, it is intended that the invention be limited only by the scope of the appended claims.

1. A method of treating or ameliorating a disorder characterized by α-synuclein toxicity or α-synuclein fibril formation, comprising administering to a subject or contacting a cell with a compound of Formula I: 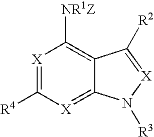
or pharmaceutically acceptable salts or derivatives thereof, wherein:
m is 1 or 2;
n is 0, 1, 2, or 3;
Each X is independently N or CH;
R¹ and Z are each independently R⁵, C(O)R⁵, COOR⁵, C(O)NR⁵R⁵, or S(O)ₘR⁵;
R² and R³ are each independently H, halo, pseudohalo, CN, SR⁵, R⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, or NR⁵C(O)C(O)NR⁵R⁶;
R⁴ is independently H; halo, pseudohalo, CN, SR⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, or NR⁵C(O)C(O)NR⁵R⁶; or optionally substituted alkyl, aryl, aralkyl, heteroaryl, or heteroaralkyl; and
each R⁵, R⁶, and R⁸ is independently H or optionally substituted alkyl, alkenyl, alkynyl, cycloalkyl, cycloalkenyl, aryl, heteroaryl, or heterocyclyl.

2. The method of claim 1, wherein the method is inhibiting or preventing α-synuclein toxicity and/or fibril formation, inhibiting or preventing α-synuclein fibril growth, and/or causing disassembly, disruption, and/or disaggregation of α-synuclein fibrils and α-synuclein-associated protein deposits, comprising administering to a mammal or contacting a cell with the compound of Formula I^{o}: 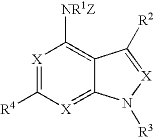
or pharmaceutically acceptable salts or derivatives thereof, wherein:
n is 0, 1, 2, or 3;
R² is H, halo, pseudohalo, (CH₂)ₙ—Y, or (CH═CH)ₙ—Y, where Y is unsubstituted or substituted aryl, heteroaryl, alkyl, or cycloalkyl;
R³ is substituted or unsubstituted alkyl, alkenyl, alkynyl, aryl, aralkyl, cycloalkyl, (CH₂)ₙ-cycloalkyl, or adamantyl;
R⁴ is H, NH₂, NR⁵R⁶, NR⁵COR⁶, or unsubstituted or substituted alkyl or aryl;
R¹, Z, R⁵, and R⁶ are independently selected from H, unsubstituted or substituted alkyl, aralkyl, aryl, alkaryl, or cycloalkyl, COR^{o}^{⁷}, where R^{o}^{⁷} is unsubstituted or substituted alkyl or aryl, SO₂R^{o}^{⁸}, where R^{o}^{⁸} is aryl or substituted aryl, and (CH₂)ₙ-cycloalkyl, where the cycloalkyl may be substituted; and
X is CH or N.

3. The method of claim 1, wherein substituents for Y are selected from the group consisting of halo, pseudohalo, alkyl, cycloalkyl, aryl, aralkyl, NO₂, alkoxy, aryloxy, arylalkyoxy, CF₃, OCF₃, CN, NR⁵R⁶, NR⁵COR⁶, (CH₂)ₙOR⁶, SR⁶, CO₂H, CO₂R⁶, CONR⁶R⁵, COR⁶, and SO₂NR⁵R⁶.

4. The method of claim 1, wherein n is 1.

5. The method of claim 1, wherein each X is N.

6. The method of claim 1, wherein R³ is selected from the group consisting of substituted or unsubstituted alkyl, cycloalkyl, aryl, and aralkyl.

7. The method of claim 1, wherein R² is selected from the group consisting of hydrogen, halo, or substituted or unsubstituted aryl, heteroaryl, aralkyl, and aralkenyl.

8. The method of claim 1, wherein R¹ and Z are each independently selected from the group consisting of hydrogen, or substituted or unsubstituted alkyl, arylcarbonyl, aralkylcarbonyl, haloarylcarbonyl, arylsulfonyl, aralkylsulfonyl, and haloarylsulfonyl.

9. The method of claim 1, wherein R¹ is H and Z is H.

10. The method of claim 1, wherein R¹ is methyl and Z is H.

11. The method of claim 1, wherein R⁴ is H.

12. The method of claim 1, wherein R⁴ is NH₂.

13. The method of claim 1, wherein the compound is selected from the compounds set forth in FIG. 1a.

14. The method of claim 2, wherein the method is treating or ameliorating one or more symptoms of a synuclein disease or synucleinopathy in a mammal, comprising administering to the mammal a compound of Formula I^{o}.

15. The method of claim 13, wherein the synuclein disease or synucleinopathy is Parkinson's disease, familial Parkinson's disease, Lewy body disease, the Lewy body variant of Alzheimer's disease, dementia with Lewy bodies, multiple system atrophy, and the Parkinsonism-dementia complex of Guam.

16. The method of claim 13, wherein the synuclein disease or synucleinopathy is associated with α-synuclein toxicity.

17. The method of claim 1, wherein the compound is represented by Formula Ib:

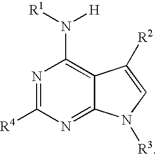

18. The method of claim 1, wherein the compound is represented by Formula Ic:

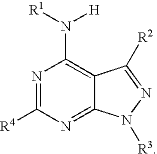

19. The method of claim 17, wherein R¹ is H.

20. The method of claim 17, wherein:
R² is H, halo, CN, NO₂, NH₂, or C₁-C₁₀ alkyl optionally substituted with 1-3 independent halo, SR⁵, OR⁵, OC(O)R⁵, NR⁵R⁵; COOR⁵, NO₂, CN, C(O)R⁵, OC(O)NR⁵R⁵, or C(O)NR⁵R⁵.

21. The method of claim 20, wherein R² is H, F, Cl, Br, CF₃, CCl₃, CN, NO₂, NH₂, or C₁-C₆ alkyl.

22. The method of claim 20, wherein R² is aryl, heteroaryl, aralkyl, or heteroaralkyl, each substituted with:
H, halo, SR⁵, OR⁵, OC(O)R⁵, NR⁵R⁵; COOR⁵, NO₂, CN, C(O)R⁵, OC(O)NR⁵R⁵, or C(O)NR⁵R⁵; or
aryl, C₁-C₁₀ alkyl, or C₂-C₁₀ alkenyl each optionally substituted with 1-3 independent aryl, halo, SR⁵, OR⁵, OC(O)R⁵, NR⁵R⁵; COOR⁵, NO₂, CN, C(O)R⁵, OC(O)NR⁵R⁵, or C(O)NR⁵R⁵.

23. The method of claim 22, wherein the optionally substituted aryl, heteroaryl, aralkyl, or heteroaralkyl groups in R² are selected from phenyl, napthyl, benzyl, phenylethylene, napthylmethylene, phenoxymethylene, napthyloxymethylene, pyridylmethylene, benzofurylmethylene, dihydrobenzofurylmethylene, benzodioxolmethylene, indanylmethylene, furyl, thienyl, pyridyl, benzothienyl, and benzofuryl.

24. The method of claim 22, wherein the optional substituents for the aryl, heteroaryl, aralkyl, or heteroaralkyl groups in R² are:
H, F, Cl, Br, OH, C₁-C₆ alkoxy, amino, C₁-C₆ alkylamino, COOH, COO—C₁-C₆ alkyl, NO₂, CN, or C(O)—C₁-C₆ alkyl; or
C₁-C₆ alkyl, C₂-C₆ alkenyl, or aryl optionally substituted with phenyl, F, Cl, Br, C₁-C₆ alkoxy, COOH, COO—C₁-C₆ alkyl, NO₂, or CN.

25. The method of claim 22, wherein R³ is H; C₁-C₁₀ alkyl or C₂-C₁₀ alkenyl each optionally substituted with 1-3 halo, CF₃, SR⁵, OR⁵, OC(O)R⁵, NR⁵R⁵; COOR⁵, NO₂, CN, C(O)R⁵, OC(O)NR⁵R⁵, C(O)NR⁵R⁵; C₃-C₁₀ cycloalkyl; or C₂-C₁₀ alkynyl.

26. The method of claim 25, wherein R³ is:
H, C₁-C₈ alkyl optionally substituted with 1-3 halo, OR⁵, NR⁵R⁵, COOR⁵, C(O)R⁵, C(O)NR⁵R⁵, C₂-C₆ alkenyl, or C₂-C₆ alkynyl; or
cyclopropyl, cyclopropylmethyl, cyclobutyl, cyclobutylmethyl, cyclopentyl, cyclopentylmethyl, cyclohexyl, or cyclohexylmethyl.

27. The method of claim 20, wherein R³ is aryl, heteroaryl, aralkyl, heteroaralkyl, heterocyclyl, or heterocyclyalkyl, each substituted with:
H, alkyl, halo, OR⁵, OC(O)R⁵, NR⁵R⁵; COOR⁵, NO₂, CN, C(O)R⁵, OC(O)NR⁵R⁵, or C(O)NR⁵R⁵; or
optionally substituted aryl, heteroaryl, or heterocyclyl.

28. The method of claim 27, wherein the aryl, heteroaryl, aralkyl, heteroaralkyl, heterocyclyl, or heterocyclyalkyl groups represented by R³ are selected from benzyl, pyridyl, pyridylmethylene, furyl, thienyl, tetrahydrofuryl, or tetrahydrothienyl.

29. The method of claim 28, wherein substituents for the aryl, heteroaryl, aralkyl, heteroaralkyl, heterocyclyl, or heterocyclyalkyl groups represented by R³ are:
H, F, Cl, Br, SR⁵, OR⁵, NR⁵R⁵; COOR⁵, NO₂, CN, C(O)R⁵; or
C₁-C₆ alkyl, C₂-C₆ alkenyl, or aryl optionally substituted with phenyl, F, Cl, Br, SR⁵, OR⁵, COOR⁵, NO₂, or CN.

30. The method of claims 20, wherein R⁴ is independently aryl; heteroaryl; C₁-C₁₀ alkyl or C₂-C₁₀ alkenyl, each optionally substituted with 1-3 independent aryl, R⁷, or heteroaryl; C₂-C₁₀ alkynyl; halo; haloalkyl; CF₃; SR⁵; OR⁵; OC(O)R⁵; NR⁵R⁵; NR⁵R⁶; COOR⁵; NO₂; CN; C(O)R⁵; C(O)C(O)R⁵; C(O)NR⁵R⁵; S(O)ₘR⁵; S(O)ₘNR⁵R⁵; NR⁵C(O)NR⁵R⁵; NR⁵C(O)C(O)R⁵; NR⁵C(O)R⁵; NR⁵(COOR⁵); NR⁵C(O)R⁸; NR⁵S(O)ₘNR⁵R⁵; NR⁵S(O)ₘR⁵; NR⁵S(O)ₘR⁸; NR⁵C(O)C(O)NR⁵R⁵; or NR⁵C(O)C(O)NR⁵R⁶.

31. The method of claim 30, wherein R⁴ is
H; OR⁵; OC(O)R⁵; NR⁵R⁵; COOR⁵; NO₂; CN; C(O)R⁵; C(O)C(O)R⁵; or C(O)NR⁵R⁵; or
C₁-C₁₀ alkyl optionally substituted with 1-3 halo, OR⁵, OC(O)R⁵, NR⁵R⁵; COOR⁵, NO₂, CN, C(O)R⁵, OC(O)NR⁵R⁵, or C(O)NR⁵R⁵.

32. The method of claim 31, wherein R⁴ is
H, CF₃, CCl₃, amino, C₁-C₆ alkoxy, COOH, COO—C₁-C₆ alkyl, OC(O)—C₁-C₆ alkyl, phenoxy, or alkylphenoxy; or
C₁-C₆ alkyl optionally substituted with amino, COOH, COO—C₁-C₆ alkyl or OC(O)—C₁-C₆ alkyl, or 1 or 2 C₁-C₆ alkoxy.

33. The method of claim 31, wherein R⁴ is an optionally substituted aryl, aralkyl, heteroaryl, or heteroaralkyl, wherein the optional substituents are halo, CF₃, SR⁵, OR⁵, OC(O)R⁵, NR⁵R⁵; COOR⁵, NO₂, CN, C(O)R⁵, OC(O)NR⁵R⁵, C(O)NR⁵R⁵, N(R⁵)C(O)R⁵, N(R⁵)(COOR⁵), or S(O)ₘNR⁵R⁵.

34. The method of claim 33, wherein the aryl, aralkyl, heteroaryl, and heteroaralkyl groups represented by R⁴ are selected from phenyl, benzyl, pyridyl, pyridylmethylene, furyl, furylmethylene, thienyl, thienylmethylene, pyrazolyl, and pyrazolylmethylene.

35. The method of claim 33, wherein the optional substituents for the aryl, aralkyl, heteroaryl, or heteroaralkyl groups represented by R⁴ are:
F, Cl, OH, amino, NO₂, C₁-C₆ alkoxy, C₁-C₆ alkyl, phenoxy, or alkylphenoxy; or
phenyl, imidazolyl, or morpholino optionally substituted with F, Cl, amino, NO₂, C₁-C₆ alkoxy, or C₁-C₆ alkyl.

36. The method of claim 1, wherein the compound is selected from the compounds in FIGS. 1a, 1b, 1c, 1d, 1e, or 1f.

37. A composition comprising a compound of Formula I as set forth in claim 1 or a compound as set forth in FIGS. 1a, 1b, 1c, 1d, 1e, or 1f, or a pharmaceutically acceptable salt or derivative thereof, and one or more of donepezil hydrochloride (Aracept), rivastigmine tartrate (Exelon), tacrine hydrochloride (Cognex) or galantamine hydrobromide (Reminyl).

38. The composition of claim 37, wherein the compound is represented by Formula I^{o} or is a compound as set forth in FIG. 1a or 1f, or a pharmaceutically acceptable salt or derivative thereof, and one or more of the following: donepezil hydrochloride (Aracept), rivastigmine tartrate (Exelon), tacrine hydrochloride (Cognex) and galantamine hydrobromide (Reminyl).

39. A method of inhibiting or preventing α-synuclein toxicity and/or fibril formation, inhibiting or preventing α-synuclein fibril growth, and/or causing disassembly, disruption, and/or disaggregation of α-synuclein fibrils and α-synuclein-associated protein deposits, comprising administering to a mammal or contacting a cell with the composition of claim 37.

40. The method of claim 39, wherein the method is inhibiting or preventing α-synuclein toxicity and/or fibril formation, inhibiting or preventing α-synuclein fibril growth, and/or causing disassembly, disruption, and/or disaggregation of α-synuclein fibrils and α-synuclein-associated protein deposits, comprising administering to a mammal or contacting a cell with the composition of claim 38, wherein the compound of the composition is represented by Formula I^{o} or is a compound as set forth in FIGS. 1a and 1f, or a pharmaceutically acceptable salt or derivative thereof.

41. The method of claim 40, wherein the method is treating or ameliorating the symptoms of a synuclein disease or synucleinopathy, comprising administering to a mammal the composition of claim 37 or 38.

42. The method of claim 41, wherein the synuclein disease or synucleinopathy is Parkinson's disease, familial Parkinson's disease, Lewy body disease, the Lewy body variant of Alzheimer's disease, dementia with Lewy bodies, multiple system atrophy, or the Parkinsonism-dementia complex of Guam.

43. The method of claim 41, wherein the synuclein disease or synucleinopathy is associated with α-synuclein toxicity.

44. The method of claim 2, wherein the method is treating or ameliorating one or more symptoms of α-synuclein toxicity in a mammal, comprising administering to the mammal a compound of Formula I^{o}.

45.-51. (canceled)

52. A compound represented by structural formula I: 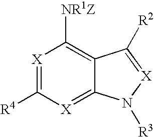
or pharmaceutically acceptable salts or derivatives thereof, wherein:
m is 1 or 2;
n is 0, 1, 2, or 3;
Each X is independently N or CH;
R¹ and Z are each independently R⁵, C(O)R⁵, COOR⁵, C(O)NR⁵R⁵, or S(O)ₘR⁵;
R² and R³ are each independently H, halo, pseudohalo, CN, SR⁵, R⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, or NR⁵C(O)C(O)NR⁵R⁶;
R⁴ is independently H; halo, pseudohalo, CN, SR⁵, OR⁵, OC(O)R⁵, NR⁵R⁵, NR⁵R⁶, COOR⁵, NO₂, C(O)R⁵, C(O)C(O)R⁵, C(O)NR⁵R⁵, S(O)ₘR⁵, S(O)ₘNR⁵R⁵, NR⁵C(O)NR⁵R⁵, NR⁵C(O)C(O)R⁵, NR⁵C(O)R⁵, NR⁵(COOR⁵), NR⁵C(O)R⁸, NR⁵S(O)ₘNR⁵R⁵, NR⁵S(O)ₘR⁵, NR⁵S(O)ₘR⁸, NR⁵C(O)C(O)NR⁵R⁵, or NR⁵C(O)C(O)NR⁵R⁶; or optionally substituted alkyl, aryl, aralkyl, heteroaryl, or heteroaralkyl; and
each R⁵, R⁶, and R⁸ is independently H or optionally substituted alkyl, alkenyl, alkynyl, cycloalkyl, cycloalkenyl, aryl, heteroaryl, or heterocyclyl,
provided that the compound is not a compound in FIGS. 1c, 1d, or 1e.

53. The compound of claim 52, wherein:
when R¹ and Z are H, then:
R² is 5-NO₂-fur-2-yl, or phenyl optionally substituted with a single 4-Cl, 4-CH₃, or 4-OCH₃; and R³ is unsubstituted phenyl, cyclohexyl, or acyclic C₁-C₄ alkyl; and the compound is in the form of a free base; then R⁴ is not H, unsubstituted C₁-C₄ alkyl, or phenyl optionally substituted with 4-Cl or 4-CH₃;
R² is CN or CH₂CN; and R³ is CH₃, or phenyl optionally substituted with 4-NO₂; then R⁴ is not CO₂-alkyl or CCl₃;
R³ is cyclopentyl, and R⁴ is unsubstituted 4-pyridyl, then R² is not CF₃; CN, Br, Cl, or NO₂;
R³ is cyclopentyl, and R⁴ is optionally substituted 4-pyridyl, then R² is not C₁-C₄ alkyl optionally substituted with F;
R³ is unsubstituted C₁-C₄ alkyl, cyclopentyl, or phenyl, and R⁴ is unsubstituted pyridyl, then R² is not unsubstituted CH₃, benzyl, or CH₂-pyrid-4-yl, and then R² is not H when the compound is in the form of a free base;
R² is H or unsubstituted C₁-C₂ alkyl, benzyl, or CH₂-pyridyl; and R⁴ is unsubstituted 4-pyridyl, then R³ is not a lone pair, C₁-C₄ alkyl optionally substituted with CO₂-alkyl, dialkylamino, or cyclopentyl; benzyl optionally substituted with Cl, CN, or CH₃; unsubstituted cyclobutyl, cyclopentyl, 3-tetrahydrofuryl, or 2-bicyclo[2.2.1]heptyl; and then R³ is not H when the compound is in the form of a free base;
R³ is H, a lone pair, cyclopentyl, 3-(5-ethyl-5H-[1,2,4]triazino[5,6-b]indolyl); unsubstituted benzyl; C₁-C₄ alkyl optionally substituted with OCH₃; phenyl optionally substituted with Cl, 3-NO₂, 4-NO₂, or 4-Me; or ribofuranose; and R⁴ is 2-furyl optionally substituted with 5-NO₂; 5-NH₂-pyrazol-4-yl optionally substituted with methyl or optionally chlorinated phenyl; phenyl optionally substituted with imidazolyl, 4-Cl, 4-OH, or 4-NO₂; C₁-C₄ alkyl optionally substituted with F or acetate; or unsubstituted benzyl; then R² is not unsubstituted C₁-C₂ alkyl, and when the compound is in the form of a free base, then R² is not H;
R³ is H or a lone pair, and R⁴ is phenyl optionally substituted with OH, NH₂, NO₂, NHC(O)NHPhSO₂F, NHC(O)PhSO₂F; fur-2-yl with an optional 5-NO₂ group, 3-NH₂-pyrazol-4-yl; C₁-C₄ alkyl optionally substituted with F or CO₂-alkyl; or unsubstituted pyridyl or benzyl; then R² is not CN, and R² is not H when the compound is in the form of a free base; and
when R³ is tert-butyl; R⁴ is H; R¹ and Z are both H or acetyl, or R¹ is H and Z is acetyl, optionally substituted SO₂-phenyl, or substituted benzoyl; then R² is not H or Br; phenyl optionally 3 or 4-substituted with OCH₃, phenoxy or benzyloxy, or substituted only with a single Cl, 4-CF₃, 4-F, 4-C₁-C₄ alkyl, or 4-phenyl; benzyl optionally substituted with Cl, F, or CH₃; unsubstituted naphthyl, CH₂-naphthyl, or OCH₂-naphthyl; or unsubstituted thien-2-yl or benzothien-2-yl.

54. The compound of claim 52, wherein:
when R¹ and Z are H, then:
R² is nitrofuryl, or phenyl optionally substituted with halo, alkyl, or alkoxy; and R³ is unsubstituted alkyl, cycloalkyl, or phenyl; then R⁴ is not H, unsubstituted alkyl, or phenyl optionally substituted with Cl or alkyl;
R² is CN or CH₂CN; and R³ is alkyl, or phenyl optionally substituted with NO₂; then R⁴ is not CO₂-alkyl or CCl₃;
R³ is cycloalkyl, and R⁴ is optionally substituted pyridyl, then R² is not CF₃; CN, Br, Cl, or NO₂, or alkyl optionally substituted with F;
R³ is unsubstituted alkyl, cycloalkyl, or phenyl, and R⁴ is unsubstituted pyridyl, then R² is not H or unsubstituted alkyl, benzyl, or CH₂-pyridyl;
R² is H or unsubstituted alkyl, benzyl, or CH₂-pyridyl; and R⁴ is unsubstituted pyridyl, then R³ is not H, a lone pair, alkyl optionally substituted with CO₂-alkyl, dialkylamino, or cycloalkyl; benzyl optionally substituted with Cl, CN, or alkyl; unsubstituted cycloalkyl, bicycloalkyl, or tetrahydrofuryl;
R² is H or unsubstituted alkyl, and R³ is H, a lone pair, cycloalkyl, a tricyclic heteroaryl substituted with alkyl; unsubstituted benzyl; C₁-C₄ alkyl optionally substituted with OCH₃; phenyl optionally substituted with Cl, NO₂, or Me; or ribofuranose; then R⁴ is not furyl optionally substituted with NO₂; NH₂-pyrazolyl optionally substituted with methyl or optionally chlorinated phenyl; phenyl optionally substituted with imidazolyl, Cl, OH, or NO₂; C₁-C₄ alkyl optionally substituted with F or acetate; or unsubstituted benzyl; and
R³ is H or a lone pair, and R² is H or CN, then R⁴ is not phenyl optionally substituted with OH, NH₂, NO₂, NHC(O)NHPhSO₂F, NHC(O)PhSO₂F; furyl optionally substituted with NO₂, NH₂-pyrazolyl; C₁-C₄ alkyl optionally substituted with F or CO₂-alkyl; or unsubstituted pyridyl or benzyl; and
when R¹ and Z are both H or acetyl, or R¹ is H and Z is acetyl, SO₂-phenyl, or optionally substituted benzoyl, R³ is tert-butyl, and R⁴ is H, then R² is not H or Br; phenyl optionally substituted with Cl, CF₃, F, C₁-C₄ alkyl, phenyl, or OCH₃, phenoxy or benzyloxy; benzyl optionally substituted with Cl, F, or CH₃; unsubstituted naphthyl, CH₂-naphthyl, or OCH₂-naphthyl; or unsubstituted thienyl or benzothienyl.

55. The compound of claim 52, wherein:
when R¹ and Z are H, then:
R² is nitrofuryl or optionally substituted phenyl; and R³ is unsubstituted alkyl, cycloalkyl, or phenyl; then R⁴ is not H, unsubstituted alkyl, or optionally substituted phenyl;
R² is CN or CH₂CN; and R³ is alkyl, or phenyl optionally substituted with NO₂; then R⁴ is not CO₂-alkyl or CCl₃;
R³ is unsubstituted alkyl, cycloalkyl, or phenyl, and R⁴ is optionally substituted pyridyl, then R² is not H oCF₃; CN, Br, Cl, NO₂, alkyl, haloalkyl, benzyl, or CH₂-pyridyl;
R² is H or unsubstituted alkyl, benzyl, or CH₂-pyridyl; and R⁴ is unsubstituted pyridyl, then R³ is not H, a lone pair, optionally substituted alkyl, dialkylamino, or cycloalkyl; optionally substituted benzyl; cycloalkyl, bicycloalkyl, or tetrahydrofuryl;
R² is H or alkyl, and R³ is H, a lone pair, cycloalkyl, a tricyclic heteroaryl substituted with alkyl; benzyl; alkyl, alkoxyalkyl; optionally substituted phenyl; or ribofuranose; then R⁴ is not optionally substituted furyl, NH₂-pyrazolyl, phenyl, alkyl or benzyl;
R³ is H or a lone pair, and R² is H or CN, then R⁴ is not an optionally substituted phenyl; furyl, pyrazolyl; alkyl, pyridyl or benzyl; and
when R¹ and Z are both H or acetyl, or R¹ is H and Z is acetyl, SO₂-phenyl, or optionally substituted benzoyl, R³ is tert-butyl, and R⁴ is H, then R² is not H or Br; optionally substituted phenyl, phenoxy, benzyloxy, benzyl, naphthyl, CH₂-naphthyl, OCH₂-naphthyl, thienyl or benzothienyl.

56. The compound of claim 52, wherein:
when R¹ and Z are H, then:
R² is nitrofuryl or optionally substituted phenyl; and R³ is alkyl, cycloalkyl, or phenyl; then R⁴ is not H, alkyl, or optionally substituted phenyl;
R² is CN or CH₂CN; and R³ is alkyl or optionally substituted phenyl; then R⁴ is not CO₂-alkyl or CCl₃;
R³ is unsubstituted alkyl, cycloalkyl, or phenyl, and R⁴ is optionally substituted pyridyl, then R² is not H, CN, Br, C₁, NO₂, alkyl, haloalkyl, benzyl, or CH₂-pyridyl;
R² is H or unsubstituted alkyl, benzyl, or CH₂-pyridyl; and R⁴ is unsubstituted pyridyl, then R³ is not H, a lone pair, dialkylamino, or optionally substituted alkyl, cycloalkyl, bicycloalkyl, benzyl, or tetrahydrofuryl;
R² is H or alkyl, and R³ is H, a lone pair, cycloalkyl, a substituted tricyclic heteroaryl, benzyl, alkyl, alkoxyalkyl; optionally substituted phenyl; or a sugar; then R⁴ is not optionally substituted furyl, pyrazolyl, phenyl, alkyl or benzyl;
R³ is H or a lone pair, and R² is H or CN, then R⁴ is not an optionally substituted phenyl, furyl, pyrazolyl, alkyl, pyridyl or benzyl; and
when R¹ and Z are both H or acetyl, or R¹ is H and Z is acetyl, SO₂-phenyl, or optionally substituted benzoyl, R³ is tert-butyl, and R⁴ is H, then R² is not H or Br; optionally substituted phenyl, phenoxy, benzyloxy, benzyl, naphthyl, CH₂-naphthyl, OCH₂-naphthyl, thienyl or benzothienyl.

57. The compound of claim 52, wherein the compound is represented by the following structural formula: 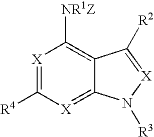
or pharmaceutically acceptable salts or derivatives thereof, wherein:
each X is independently N or CH; and
n is 0, 1, 2, or 3;
R² is H, halo, pseudohalo, (CH₂)ₙ—Y, or (CH═CH)ₙ—Y, where Y is unsubstituted or substituted aryl, heteroaryl, alkyl, or cycloalkyl;
R³ is substituted or unsubstituted alkyl, alkenyl, alkynyl, aryl, aralkyl, cycloalkyl, (CH₂)ₙ-cycloalkyl, or adamantyl;
R⁴ is H, NH₂, NR⁵R⁶, NR⁵COR⁶, or unsubstituted or substituted alkyl or aryl;
R¹, Z, R⁵, and R⁶ are independently selected from H, unsubstituted or substituted alkyl, aralkyl, aryl, alkaryl, or cycloalkyl, COR^{o}^{⁷}, where R^{o}^{⁷} is unsubstituted or substituted alkyl or aryl, SO₂R^{o}^{⁸}, where R^{o}^{⁸} is aryl or substituted aryl, and (CH₂)ₙ-cycloalkyl, where the cycloalkyl may be substituted;

58. The compound of claim 52, wherein R¹ and Z are independently H, C₁-C₆ alkyl, C(O)—C₁-C₆ alkyl, C(O)-aryl, S(O)ₘ—C₁-C₆ alkyl or S(O)ₘ-aryl, wherein each C₁-C₆ alkyl and aryl represented in R¹ and Z is optionally substituted with C₁-C₆ alkyl, C₁-C₆ alkoxy, or halo.

59. The compound of claim 52, wherein Z is H and R¹ is C₁-C₆ alkyl, C(O)—C₁-C₆ alkyl; or C(O)-phenyl or S(O)₂-phenyl optionally substituted with C₁-C₆ alkyl, F, or Cl.

60. The compound of claim 52, wherein R¹ and Z are each H.

61. The compound of claim 52, wherein the compound is represented by one of structural formulas:

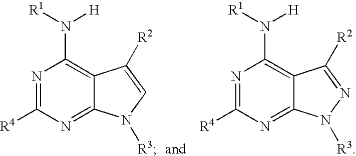

62. The compound of claim 52, wherein R² is phenyl, napthyl, benzofuryl, benzothienyl, furyl, or thienyl, each optionally substituted with:
halo, CN, amino, alkylamino, C₁-C₆ hydroxyalkyl, S—C₁-C₆ alkyl, C₁-C₆ alkoxy, C₁-C₆ haloalkoxy, COOH, COO—C₁-C₆ alkyl, C(O)—C₁-C₆ alkyl, or C₃-C₆ cycloalkyl; or
optionally halogenated aryl, aralkyl, O-aryl, or O-aralkyl.

63. The compound of claim 62, wherein R² is phenyl, napthyl, benzofuryl, benzothienyl, furyl, thienyl, fluoronapthyl, benzyloxyphenyl, (chlorobenzyl)oxyphenyl, hydroxymethylphenyl, cyclohexylphenyl, chorophenyl, cyanophenyl, carboxyl phenyl, alkyl carboxyl phenyl, alkanoyl phenyl, alkylamino phenyl, trifluoromethoxyphenyl, alkoxyphenyl, phenoxyphenyl, biphenyl, or alkyl-S-phenyl.

64. The compound of claim 63, wherein R² is aralkyl, aralkenyl, or heteroaralkyl, each optionally substituted with halo, CN, amino, alkylamino, S—C₁-C₆ alkyl, C₁-C₆ alkoxy, C₁-C₆ haloalkoxy, C₁-C₆ haloalkyl, C₂-C₆ alkynyl, aryl, haloaryl, or heteroaryl.

65. The compound of claim 64, wherein R² is CH₂, CH(CH₃), CH═CH, or CH₂CH₂, each substituted with phenyl, naphthyl, tetrahydronaphthyl, pyridyl, indanyl, benzofuryl, benzodioxolyl, dihydrobenzofuranyl, or tetrahydronaphthyl, wherein each phenyl, napthyl, tetrahydronaphthyl, pyridyl, indanyl, benzofuryl, benzodioxolyl, dihydrobenzofuranyl, or tetrahydronaphthyl in R² is optionally substituted with one or two substituents selected from the group consisting of F, Cl, CF₃; C₁-C₆ alkyl, C₁-C₆ alkoxy, acetylenyl, CN, alkylamino, and phenyl.

66. The compound of claim 64, wherein R² is CH(CH₃)-phenyl, CH═CH-phenyl, CH₂CH₂-phenyl, CH₂-naphthyl, CH₂— (methylnaphthyl), CH₂— (fluoronaphthyl), CH₂-pyridyl, CH₂-indanyl, CH₂-benzofuryl, CH₂-benzodioxolyl, CH₂-dihydrobenzofuranyl, CH₂-tetrahydronaphthyl, dichlorobenzyl, (chloro,trifluoromethyl)benzyl, (fluoro,trifluoromethyl)benzyl, (fluoro,chloro)benzyl, dimethylbenzyl, (methyl,fluoro)benzyl, dimethoxybenzyl, (acetylenyl)benzyl, cyanobenzyl, (dimethylamino)benzyl, methoxybenzyl, or phenylbenzyl.

67. The compound of claim 62, wherein R³ is optionally substituted aryl; C₁-C₁₀ alkyl optionally substituted with aryl or C₃-C₁₀ cycloalkyl; C₃-C₁₀ cycloalkyl; C₂-C₁₀ alkenyl, or C₂-C₁₀ alkynyl.

68. The compound of claim 67, wherein R³ is propenyl, propynyl, benzyl, cyclobutyl, cyclopropylmethyl, 2,2-dimethylpropyl, cyclohexyl, cyclopentyl, cyclopropyl, phenylethylene, ethyl, 2-propyl, methyl, phenyl, nitrophenyl, sec-butyl, or tert-butyl.

69. The compound of claim 64, wherein the compound is represented by the following structural formula: 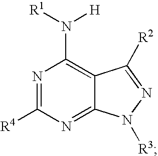
wherein R⁴ is independently amino, alkylamino, or aryl, heteroaryl, or C₁-C₁₀ alkyl optionally substituted with halo, CF₃, O—C₁-C₆ alkyl, or aryloxy.

70. The compound of claim 69, wherein R⁴ is pyridyl, C₁-C₆ alkoxy-C₁-C₆ alkyl, (C₁-C₆ alkyl)phenoxy-C₁-C₆ alkyl, C₁-C₆ alkyl, amino, or halophenyl.

71. The compound of claim 70, wherein R⁴ is pyridyl, CH(OCH₂CH₃)₂, tert-butyl-phenyoxymethylene, methyl, ethyl, amino, or chlorophenyl.

72. The compound of claim 64, wherein the compound is represented by the following structural formula:

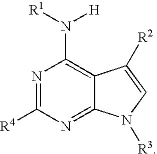

73. The compound of claim 72, wherein R⁴ is pyridyl or C₁-C₆ alkyl.

74. The compound of claim 73, wherein R⁴ is pyridyl, methyl, or ethyl.

75. The compound of claim 52, wherein the compound is selected from the compounds in FIGS. 1a and 1b.

76. A compound, or a pharmaceutically acceptable salt or derivative thereof, having a structure as set forth in FIGS. 1a, 1b, and 1f.

77. The compound of claim 76 or a pharmaceutically acceptable salt or derivative thereof, having a structure set forth in FIG. 1a.

78. The compound of claim 76, wherein the compound is one of: [chemical structure]
